(19) United States
(12) Patent Application Publication
(10) Pub. No.: US 2012/0322827 A1
(43) Pub. Date: Dec. 20, 2012

(54) COMPOUNDS AND METHODS

(76) Inventors: Erkan Baloglu, Cambridge, MA (US); Shomir Ghosh, Cambridge, MA (US); Mercedes Lobera, Cambridge, MA (US); Darby Schmidt, Cambridge, MA (US)

(21) Appl. No.: 13/522,051

(22) PCT Filed: Jan. 13, 2011
(86) PCT No.: PCT/US11/21099

§ 371 Date: Jul. 13, 2012

Related U.S. Application Data
(60) Provisional application No. 61/294,643, filed on Jan. 13, 2010.

(51) Int. Cl. A61K 31/4245; C07D 413/12; A61K 31/4196; A61K 31/422; C07D 409/12; C07D 413/14; A61K 31/454

(52) U.S. Cl. 514/326; 548/131; 514/364; 546/209; 548/235; 514/374; 548/266.6; 514/383

(57) ABSTRACT

Disclosed are compounds having the formula:

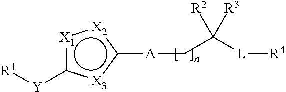

wherein X₁, X₂, X₃, R¹, R², R³, R⁴, Y, A, n and L are as defined herein, and methods of making and using the same.

### BACKGROUND OF THE INVENTION

1. Field of the Invention

The present invention relates to compounds that inhibit histone deacetylase (HDAC) enzymes, the preparation of these compounds, the use of these compounds in the treatment of diseases or conditions ameliorated by inhibition of HDAC activity and pharmaceutical compositions comprising these compounds.

2. Background of the Invention

Chromatin organization involves DNA wound around histone octamers that form nucleosomes. Core histones with N-terminal tails extending from compact nucleosomal core particles can be acetylated or deacetylated at epsilon lysine residues affecting histone-DNA and histone-non-histone protein interactions. Histone deacetylases (HDACs) catalyze the deacetylation of histone and non-histone proteins and play an important role in epigenetic regulation. There are currently 18 known HDACs that are organized into three classes: class I HDACs (HDAC1, HDAC2, HDAC3, HDAC8 and HDAC11) are mainly localized to the nucleus; class II HDACs (HDAC4, HDAC5, HDAC6, HDAC7, HDAC9 and HDAC10), which shuttle between the nucleus and the cytoplasm; and class III HDACs (SIRT1-7), whose cellular localization includes various organelles.

Class II HDACs are further characterized as class IIa HDACs and class IIb HDACs.

HDAC9 is class IIa histone deacetylase highly expressed in human Tregs. HDAC9 deficiency: 1) increases Foxp3 expression (and other Treg markers), 2) increases Foxp3 and histone 3 acetylation, 3) increases Foxp3 DNA binding, 4) increases Treg numbers, 5) increases suppressive activity in vitro and in vivo, and 6) ameliorates murine colitis. Tregs which are deficient in HDAC9 induce permanent tolerance of fully mismatched cardiac allografts. In addition, HDAC9 inhibitors maybe useful for treatment of diseases and disorders associated with abnormal cell proliferation, differentiation and survival, e.g breast and prostate tumors.

Preliminary data shows that targeting HDAC7, a class IIa histone deacetylase, enhances Treg suppression in vitro and in vivo. HDAC7 enhances FOXP3+ Treg function and induces long-term allograft survival.

Inhibition of HDAC6, a class IIb HDAC, has been shown to increase Treg suppressive function in vitro along with increased expression of FOXP3 protein and Treg associated genes including CTLA, IL-10, TNR18. HDAC6 inhibition in vivo decreased severity of colitis in the dextran sodium sulphate-induced colitis model and the CD4₊CD62L_{high} adoptive transfer model of colitis. In addition, inhibition of HDAC6 with a subtherapeutic dose of rapamycin led to prolonged cardiac allograft survival.

Based on the above evidence, an orally available small molecule selective inhibitor of Class II HDAC activity (more specifically HDAC9 or HDAC7 or HDAC6) is expected to modulate autoimmune diseases through expansion and enhancement of Treg activity.

Inhibition of other Class II HDAC's for example HDAC4 and 5 impair myogenesis by modulating the stability and activity of HDAC-MEF2 complexes and maybe potentially useful for the treatment of muscle and heart diseases including cardiac hypertrophy and heart failure. Also, inhibition of Class II HDAC activity represents a novel approach for disrupting or intervening in cell cycle regulation.

Class II HDAC inhibitors have therapeutic potential in the study and/or treatment of diseases or conditions ameliorated by modulating HDAC activity (in particular, cell proliferative diseases (such as cancer), diabetes (type I and/or type II diabetes), inflammation, cardiac disease, obesity, stroke, epilepsy, depression, immunological disease or viral or fungal infection.

Many HDAC inhibitors, however, inhibit all HDAC isoforms. It would be advantageous to identify HDAC inhibitors that inhibited one or more but not all HDAC isoforms.

### SUMMARY OF THE INVENTION

The invention is directed to novel HDAC inhibitors. Specifically, the invention is directed to a compound according to Formula I:

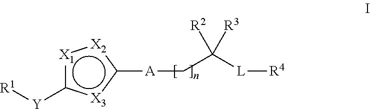

wherein:

R¹ is halo(C₁-C₄)alkyl, wherein said halo(C₁-C₄)alkyl contains at least 2 halo groups (R¹ is di-halo(C₁-C₄)alkyl);

Y is a bond and X₁ is 0, X₂ is N or CH and X₃ is N or NH,

or Y is —C(O)— and X₁ and X₂ are CH or N, X₃ is O or S,

or Y is —C(O)— and X₁ is O, X₂ is CH or N, and X₃ is CH or N;

n is 0-4;

A is —C(═O)NR^{X}—, —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —((C₁-C₆)alkyl)SO₂—, —SO₂NR^{X}—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—;

when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,

when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then Z is —C(═O)NR^{X}—, —NR^{X}C(═O)NR^{X}, —SO₂NR^{X}—, —NHCH(CF₃)—, —CH(CF₃)NH—, —CH(CF₃)—, —(C₁-C₄)alkyl-, —NR^{X}—, or —(C₁-C₃)alkyl-NR^{X}—, and

when n is 1-4, R² is selected from —NR^{A}R^{B}, —(C₁-C₄)alkyl-NR^{A}R^{B}, —CONR^{A}R^{B}, —(C₁-C₄)alkyl-CONR^{A}R^{B}, —CO₂H, —(C₁-C₄)alkyl-CO₂H, hydroxyl, hydroxy(C₁-C₄)alkyl-, (C₁-C₃)alkoxy, and (C₁-C₃)alkoxy(C₁-C₄)alkyl-, and R³ is selected from H and optionally substituted (C₁-C₄)alkyl, aryl(C₁-C₄)alkyl-, and (C₃-C₇)cycloalkyl(C₁-C₄)alkyl-,

wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, —NR^{A}R^{A}, —((C₁-C₄)alkyl)NR^{A}R^{A}, and hydroxyl;

or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, and —NR^{Y}C(═O)NR^{Y}R^{Y}; and

L is 5-6 membered heteroaryl or phenyl which is substituted by R⁴ and is optionally further substituted,

wherein when L is further substituted, L is substituted by 1 or 2 substituents independently selected from halogen, cyano and (C₁-C₄)alkyl;

R⁴ is H, (C₁-C₄)alkyl, halo, halo(C₁-C₄)alkyl, (C₁-C₄)alkoxy, ((C₁-C₄)alkyl)((C₁-C₄)alkyl)N(C₁-C₄)alkoxy, ((C₁-C₄)alkyl)((C₁-C₄)alkyl)N(C₁-C₄)alkyl-, (C₁-C₄)haloalkoxy-, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,

wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};

wherein each R^{A} is independently selected from H and (C₁-C₄)alkyl;

R^{B} is H, (C₁-C₄)alkyl, halo(C₁-C₄)alkyl, —C(═O)(C₁-C₄)alkyl, —C(═O)((C₁-C₄)alkyl, —C(═O)NH₂, —C(═O)NH(C₁-C₄)alkyl, —C(═O)N((C₁-C₄)alkyl)((C₁-C₄)alkyl), —SO₂(C₁-C₄)alkyl, or R^{A} and R^{B} taken together with the atom to which they are attached form a 4-6 membered heterocyclic ring, optionally containing one additional heteroatom selected from N, O and S and optionally substituted by (C₁-C₄)alkyl;

R^{C} is H, (C₁-C₄)alkyl, phenyl, 5-6 membered heterocycloalkyl, or 5-6 membered heteroaryl, or R^{A} and R^{C} taken together with the atom to which they are attached form a 4-8 membered heterocyclic ring, optionally containing one additional heteroatom selected from N, O and S and optionally substituted by (C₁-C₄)alkyl;

each R^{X} is independently selected from H, (C₁-C₆)alkyl, and optionally substituted (C₂-C₆)alkyl, where said optionally substituted (C₂-C₆)alkyl is optionally substituted by hydroxyl, cyano, amino, (C₁-C₄)alkoxy, (C₁-C₄)alkyl)NH—, or ((C₁-C₄)alkyl)((C₁-C₄)alkyl)N—; and

each R^{Y} is independently selected from H, (C₁-C₄)alkyl, phenyl, and —(C₁-C₄)alkylphenyl;

provided that when Y is —C(O)— and A is —C(═O)NR^{X}— or —SO₂NR^{X}—, then at least one of R² and R³ is not H (either one or both of R² and R³ is/are not H);

or a salt, particularly a pharmaceutically acceptable salt, thereof, and is further directed to a pharmaceutical composition comprising the compound of Formula I, or a salt thereof, a method of inhibiting HDAC by contacting a HDAC with the compound of Formula I or a salt thereof, and a method of treating a subject having a disease or disorder mediated by inhibition of a HDAC comprising administering the compound of Formula I, or a salt thereof, or a pharmaceutical composition comprising the compound of Formula I, or a salt thereof, to the subject.

In one embodiment, a compound of Formula I excludes 2,2,2-trifluoro-1-[5-[[methyl(phenylmethyl)amino]methyl]-2-thienyl]-ethanone, 2,2,2-trifluoro-1-[5-[[[(1R)-1-phenylethyl]amino]methyl]-2-thienyl]-ethanone, N-methyl-2-phenyl-N-(2-(5-(trifluoromethyl)-1,2,4-oxadiazol-3-yl)ethyl)acetamide, N-methyl-2-phenyl-N-(2-(5-(trichloromethyl)-1,2,4-oxadiazol-3-yl)ethyl)acetamide, N-[2-[5-(dichloromethyl)-1,2,4-oxadiazol-3-yl]ethyl]-N-methyl-benzeneacetamide, N-[2-(3,4-dimethoxyphenyl)ethyl]-5-(trifluoromethyl)-1,2,4-oxadiazole-3-acetamide, and N-(phenyl methyl)-5-(trifluoromethyl)-1,2,4-oxadiazole-3-methanamine, or a salt thereof, particularly a pharmaceutical salt thereof.

The invention is further directed to a pharmaceutical composition comprising a compound of the invention. The invention is still further directed to methods of inhibiting HDAC enzymes and treatment of conditions associated therewith using a compound of the invention or a pharmaceutical composition comprising a compound of the invention.

### DETAILED DESCRIPTION OF THE INVENTION

The alternative definitions for the various groups and substituent groups of Formula I provided throughout the specification are intended to particularly describe each compound species disclosed herein, individually, as well as groups of one or more compound species. The scope of this invention includes any combination of these group and substituent group definitions.

In one embodiment of this invention, R¹ is a fluoro-alkyl group containing at least 2 fluoro groups (atoms). In another embodiment, R¹ is a (C₁-C₂)alkyl group containing at least 2 fluoro groups. In a specific embodiment, R¹ is CHF₂ or CF₃; more specifically, R¹ is CF₃

In selected embodiments, when Y is a bond, X₁, X₂, and X₃, taken together with the atoms to which they are attached, form an oxadiazolyl (X₁ is O, X₂ and X₃ are N) or and oxazolyl (X₁ is O, X₂ is CH, X₃ is N) ring moiety. In specific embodiments, when Y is a bond, X₁, X₂, and X₃, taken together with the atoms to which they are attached form an oxadiazolyl ring moiety.

In selected embodiments, when Y is —C(O)—, X₁, X₂, and X₃, taken together with the atoms to which they are attached, form an thiazolyl (X₃ is S, X₁ is CH and X₂ is N or X₃ is S, X₁ is N and X₂ is CH), oxazolyl (X₃ is 0, X₁ is CH and X₂ is N or X₃ is O, is N and X₂ is CH), thienyl (X₁ and X₂ are CH, X₃ is S) or furanyl (X₁ and X₂ are CH, X₃ is O) ring moiety. In specific embodiments, when Y is —C(O)—, X₁, X₂, and X₃, taken together with the atoms to which they are attached form a thienyl, thiazolyl or oxazolyl ring moiety, more specifically a thienyl moiety.

In selected embodiments, when Y is —C(O)—, X₁, X₂, and X₃, taken together with the atoms to which they are attached, form a furanyl or furyl (X₁ is O, X₂ and X₃ are CH), oxazolyl (X₁ is O, X₂ is CH, and X₃ is N), isoxazolyl (X₁ is O, X₂ is N, and X₃ is CH), or oxadiazolyl (X₁ is O, X₂ and X₃ are N) ring moiety. In specific embodiments, when Y is —C(O)—, X₁, X₂, and X₃, taken together with the atoms to which they are attached form a furanyl (furyl) ring moiety.

The invention is further directed to a compound of Formula (I-a):

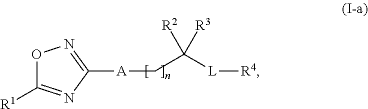

wherein R¹, R², R³, R⁴, A, n and L are as defined herein.

The invention is still further directed to a compound of Formula (I-b):

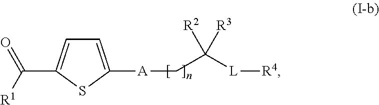

wherein R¹, R², R³, R⁴, A, Z, n and L are as defined herein.

The invention is further directed to a compound of Formula (I-c):

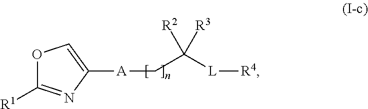

wherein R¹, R², R³, R⁴, A, n and L are as defined herein.

The invention is still further directed to a compound of Formula (I-d), (I-e), (I-f), (I-g) or (I-h):

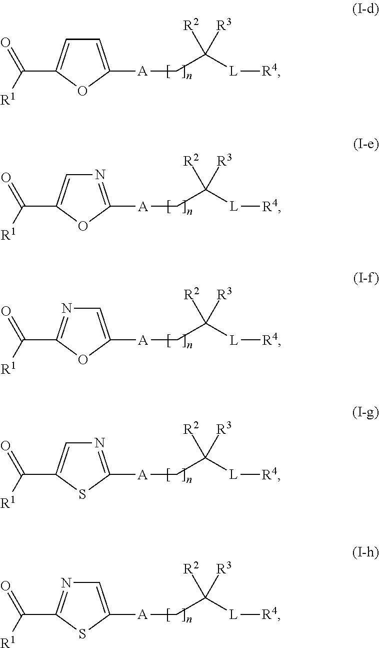

wherein R¹, R², R³, R⁴, A, n and L are as defined herein.

The invention is still further directed to a compound of Formula (I-i), (I-j), (I-k), or (I-l):

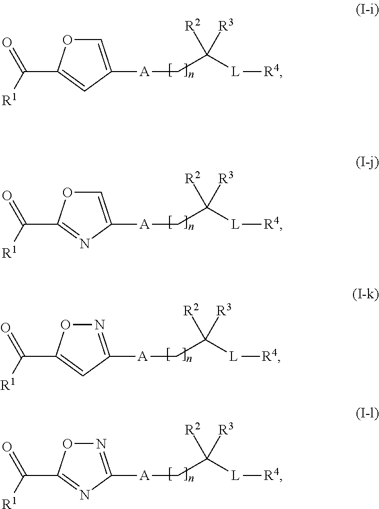

wherein R¹, R², R³, R⁴, A, n and L are as defined herein.

In another embodiment of this invention, A is —((C₁-C₆)alkyl)C(═O)NR^{X}—, —((C₁-C₆)alkyl)SO₂NR^{X}—, —((C₁-C₆)alkyl)NR^{X}C(═O)NR^{X}—, or —((C₁-C₆)alkyl)NR^{X}C(═O)—; particularly —((C₁-C₆)alkyl)C(═O)NR^{X}—, —((C₁-C₆)alkyl)SO₂NR^{X}—, or —(C₁-C₆)alkyl)NR^{X}C(═O)—. In another embodiment of this invention, A is —((C₁-C₄alkyl)C(═O)NR^{X}—, —((C₁-C₄)alkyl)SO₂NR^{X}—, —((C₁-C₄)alkyl)NR^{X}C(═O)NR^{X}— or —((C₁-C₄)alkyl)NR^{X}C(═O)—; particularly —((C₂-C₄)alkyl)C(═O)NR^{X}—, —((C₂-C₄)alkyl)SO₂NR^{X}—, or —((C₂-C₄)alkyl)NR^{X}C(═O)—. In specific embodiments, A is —CH₂CH₂C(═O)NH—, —CH₂CH₂CH₂C(═O)NH—, —CH₂CH₂CH₂SO₂NH—, —CH₂CH₂C(CH₃)₂C(═O)NH—, —CH₂CH₂C(CH₃)₂SO₂NH— or —CH₂CH₂CH₂CH₂C(═O)NH—.

In another embodiment, each R^{X} is independently selected from H, (C₁-C₄)alkyl, or optionally substituted (C₂-C₄)alkyl, where said optionally substituted (C₂-C₄)alkyl is optionally substituted by hydroxyl, cyano, amino, (C₁-C₄)alkoxy, (C₁-C₄)alkyl)NH—, or ((C₁-C₂)alkyl)((C₁-C₂)alkyl)N—. In other embodiments, each Rx is independently selected from H, methyl, ethyl, tert-butyl, hydroxyethyl-, methoxymethyl-, cyanoethyl-, N-methylaminoethyl- or dimethylaminoethyl-. In particular embodiments, each Rx is independently H or methyl. In specific embodiments, R^{X} is H.

In another embodiment of this invention, n is 0-4; particularly 0-3. In specific embodiments, n is 1.

In another embodiment of this invention, R² and R³ taken together with the atom to which they are connected form an optionally substituted 4, 5, or 6 membered cycloalkyl or heterocycloalkyl group, wherein said heterocycloalkyl group contains 1 heteroatom selected from N, O and S and said optionally substituted cycloalkyl or heterocycloalkyl group is optionally substituted by a substituent selected from (C₁-C₄)alkyl, halo(C₁-C₄)alkyl, halogen, cyano, aryl(C₁-C₂)alkyl-, (C₃-C₆)cycloalkyl(C₁-C₂)alkyl-, —NR^{Ya}R^{Yb}, —C(═O)OR^{Ya}, —C(═O)NR^{Ya}R^{b}, —NR^{Yb}C(═O)R^{Ya}, —SO₂NR^{Ya}R^{Yb}, —NR^{Yb}SO₂R^{Ya}, —OC(═O)NR^{Ya}R^{Yb}, —NR^{Yb}C(═O)OR^{Ya}, —NR^{Yb}C(═O)NR^{Ya}R^{Yb}, where R^{Ya} is selected from H, (C₁-C₄)alkyl and —(C₁-C₄)alkylphenyl and each R^{Yb} is independently selected from H and (C₁-C₄)alkyl.

In specific embodiments of this invention, R² and R³ taken together with the atom to which they are connected form an optionally substituted 4, 5 or 6 membered cycloalkyl or heterocycloalkyl group, wherein said heterocycloalkyl group contains 1 heteroatom selected from N and O and said optionally substituted cycloalkyl or heterocycloalkyl group is optionally substituted by a substituent selected from (C₁-C₄)alkyl, aryl(C₁-C₂)alkyl-, and (C₃-C₆)cycloalkyl(C₁-C₂)alkyl-.

In specific embodiments of this invention, R² and R³ taken together with the atom to which they are connected form a tetrahydropyranyl or a piperidinyl group, which tetrahydropyranyl or piperidinyl may be optionally substituted by a (C₁-C₂)alkyl or benzyl group. In a more specific embodiment, R² and R³ taken together with the atom to which they are connected form a tetrahydropyranyl or an N-methyl-piperidinyl group.

In another embodiment of this invention, (for any value of n) R² and R³ are independently selected from H and optionally substituted (C₁-C₄)alkyl, phenyl(C₁-C₂)alkyl-, and (C₃-C₆)cycloalkyl(C₁-C₂)alkyl-, provided that when Y is —C(O)— A is not —C(═O)NR^{X}— or —SO₂NR^{X}—.

In an further embodiment, (for any value of n) R² and R³ are not H; that is, R² and R³ are independently selected from optionally substituted (C₁-C₄)alkyl, phenyl(C₁-C₂)alkyl-, and (C₃-C₆)cycloalkyl(C₁-C₂)alkyl-.

In a still further embodiment, (for any value of n) one of R² and R³ is H, and the other of R² and R³ is selected from optionally substituted (C₁-C₄)alkyl, phenyl(C₁-C₂)alkyl-, and (C₃-C₆)cycloalkyl(C₁-C₂)alkyl-, provided that when Y is —C(O)— A is not —C(═O)NR^{X}— or —SO₂NR^{X}—.

In another embodiment of this invention, (for any value of n) R² is selected from H and optionally substituted (C₁-C₄)alkyl, phenyl(C₁-C₂)alkyl-, and (C₃-C₆)cycloalkyl(C₁-C₂)alkyl- and R³ is selected from H and methyl, provided that when Y is —C(O)— and A is —C(═O)NR^{X}— or —SO₂NR^{X}—, then at least one of R² and R³ is not H, that is, both of R² and R³ are not H or one of R² and R³ is not H. In a further embodiment, (for any value of n) R² is selected from optionally substituted (C₁-C₄)alkyl, phenyl(C₁-C₂)alkyl-, and (C₃-C₆)cycloalkyl(C₁-C₂)alkyl- and R³ is methyl. In a still further embodiment, (for any value of n) R² is selected from optionally substituted (C₁-C₄)alkyl, phenyl(C₁-C₂)alkyl-, and (C₃-C₆)cycloalkyl(C₁-C₂)alkyl- and R³ is H or methyl (provided that when Y is —C(O), A is not —C(═O)NR^{X}— or —SO₂NR^{X}—). In specific embodiments of this invention, R² and R³ are independently selected from H and methyl (provided that when Y is —C(O), A is not —C(═O)NR^{X}— or —SO₂NR^{X}—). In more specific embodiments, both R² and R³ are methyl.

In another embodiment of this invention, when n is 1-4, R² is selected from amino, (C₁-C₄)alkylamino, ((C₁-C₃)alkyl)((C₁-C₃)alkyl)amino, amino(C₁-C₄)alkyl, (C₁-C₃)alkylamino(C₁-C₄)alkyl, ((C₁-C₃)alkyl)((C₁-C₃)alkyl)amino(C₁-C₄)alkyl, (substituted(C₁-C₃)alkyl)((C₁-C₃)alkyl)amino(C₁-C₄)alkyl (where said (substituted (C₁-C₃)alkyl moiety is substituted by —C(═O)OH, —C(═O)O(C₁-C₄)alkyl, or 1-8 fluoro groups), aminocarbonyl(C₁-C₄)alkyl, (C₁-C₃)alkylaminocarbonyl(C₁-C₄)alkyl, ((C₁-C₃)alkyl)((C₁-C₃)alkyl)aminocarbonyl(C₁-C₄)alkyl, hydroxyl, hydroxy(C₁-C₄)alkyl-, (C₁-C₄)alkoxy, and (C₁-C₄)alkoxy(C₁-C₄)alkyl- and R³ is selected from H and optionally substituted (C₁-C₄)alkyl, aryl(C₁-C₄)alkyl-, and (C₃-C₇)cycloalkyl(C₁-C₄)alkyl-, when A is —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —SO₂—, —((C₁-C₆)alkyl)SO₂—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—.

In yet another embodiment of this invention, when n is 1-4, R² is selected from amino, hydroxyl, and (C₁-C₄)alkoxy, and R³ is selected from H and optionally substituted (C₁-C₄)alkyl, phenyl(C₁-C₂)alkyl-, and (C₃-C₆)cycloalkyl(C₁-C₂)alkyl-, when A is —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —SO₂—, —((C₁-C₆)alkyl)SO₂—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—.

In another embodiment, n is 1-3, R² is hydroxyl and R³ is H or methyl; more specifically, n is 1, R² is hydroxyl and R³ is H or methyl, when A is —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —SO₂—, —((C₁-C₆)alkyl)SO₂—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—.

In a further embodiment of this invention, when n is 1-4, and A is —C(═O)NR^{X}— or —SO₂NR^{X}—, —R² is selected from amino, (C₁-C₄)alkylamino, ((C₁-C₃)alkyl)((C₁-C₃)alkyl)amino, amino(C₁-C₄)alkyl, (C₁-C₃)alkylamino(C₁-C₄)alkyl, ((C₁-C₃)alkyl)((C₁-C₃)alkyl)amino(C₁-C₄)alkyl, (substituted(C₁-C₃)alkyl)((C₁-C₃)alkyl)amino(C₁-C₄)alkyl (where said (substituted (C₁-C₃)alkyl moiety is substituted by —C(═O)OH, —C(═O)O(C₁-C₄)alkyl, or 1-8 fluoro groups), aminocarbonyl(C₁-C₄)alkyl, (C₁-C₃)alkylaminocarbonyl(C₁-C₄)alkyl, ((C₁-C₃)alkyl)((C₁-C₃)alkyl)aminocarbonyl(C₁-C₄)alkyl, hydroxyl, hydroxy(C₁-C₄)alkyl-, (C₁-C₄)alkoxy, and (C₁-C₄)alkoxy(C₁-C₄)alkyl- and R³ is selected from optionally substituted (C₁-C₄)alkyl, aryl(C₁-C₄)alkyl-, and (C₃-C₇)cycloalkyl(C₁-C₄)alkyl-.

In another embodiment, when A is —C(═O)NR^{X}— or —SO₂NR^{X}—, n is 1-3, R² is hydroxyl and R³ is methyl. In yet another embodiment, when A is —C(═O)NR^{X}— or —SO₂NR^{X}—, n is 1, R² is hydroxyl and R³ is methyl.

In another embodiment of this invention, L is 5-6 membered heteroaryl or phenyl group which is substituted by R⁴ and is optionally further substituted by 1 substituent selected from halogen, cyano and (C₁-C₄)alkyl. In another embodiment, L is thiazolyl, thienyl, triazolyl, oxazolyl, or phenyl which is substituted by R⁴ and is optionally further substituted by a methyl group. In specific embodiments, L is thiazolyl or oxazolyl substituted only by R⁴.

In other embodiments, R⁴ is H, (C₁-C₄)alkyl, (C₁-C₄)haloalkyl, (C₁-C₄)alkoxy, ((C₁-C₄)alkyl)((C₁-C₄)alkyl)N(C₁-C₄)alkoxy, (C₁-C₄)alkylamino, optionally substituted phenyl, or optionally substituted 5-6 membered heteroaryl. In another embodiment, R⁴ is H, methyl, phenyl, 4-chlorophenyl, 4-fluorophenyl, 3,5-difluorophenyl, 4-cyanophenyl, 4-methoxyphenyl, pyrid-2-yl, pyrid-3-yl, or pyrid-4-yl. In specific embodiments, R⁴ is phenyl, 4-chlorophenyl, or 4-fluorophenyl.

As used herein, the term “alkyl” represents a saturated, straight or branched hydrocarbon moiety, which may be unsubstituted or substituted by one, or more of the substituents defined herein. Exemplary alkyls include, but are not limited to methyl (Me), ethyl (Et), n-propyl, isopropyl, n-butyl, isobutyl, sec-butyl, t-butyl, n-pentyl, iso-pentyl (3-methyl-butyl), neo-pentyl (2,2-dimethylpropyl), etc. The term “C₁-C₄” refers to an alkyl containing from 1 to 4 carbon atoms.

When the term “alkyl” is used in combination with other substituent groups, such as “haloalkyl” or “cycloalkyl-alkyl” or “arylalkyl”, the term “alkyl” is intended to encompass a divalent straight or branched-chain hydrocarbon radical. For example, “arylalkyl” is intended to mean the radical-alkylaryl, wherein the alkyl moiety thereof is a divalent straight or branched-chain carbon radical and the aryl moiety thereof is as defined herein, and is represented by the bonding arrangement present in a benzyl group (—CH₂-phenyl).

In addition, the term “alkyl” may be used to define a divalent substituent, such as a group bonded to two other groups. In this instance, the term “alkyl” is intended to encompass a divalent straight or branched-chain hydrocarbon radical. For example, “pentyl” is intended to represent a pentylene diradical—wherein the pentyl moiety is any one of a divalent straight (—CH₂CH₂CH₂CH₂CH₂—) or branched (—CH₂CH(CH₃)CH₂CH₂—, —CH₂CH₂CH(CH₂CH₃)—, —CH₂CH₂C(CH₃)₂—) chain 5-carbon radical.

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein.

Generally, in the compounds of this invention, aryl is phenyl.

Heterocyclic groups may be heteroaryl or heterocycloalkyl groups. “Heterocycloalkyl” represents a group or moiety comprising a stable, non-aromatic, monovalent monocyclic or bicyclic radical, which is saturated or partially unsaturated, containing 3 to 10 ring atoms, which includes 1 to 4 heteroatoms selected from nitrogen, oxygen and sulfur, and which may be unsubstituted or substituted by one or more of the substituents defined herein. The heterocycloalkyl may be attached by any atom of the monocyclic or bicyclic radical which results in the creation of a stable structure. This term encompasses bicyclic heterocycloalkyl moieties where the rings are joined at two atoms per ring, as exemplified by the bonding arrangement in 2,5-diazabicyclo[2.2.1]heptyl, 2-azabicyclo[2.2.1]heptyl, 2-oxa-5-azabicyclo[2.2.1]heptyl, 7-oxa-2-azabicyclo[2.2.1]heptyl, 2-thia-5-azabicyclo[2.2.1]heptyl,7-azabicyclo[2.2.1]heptyl, 2,6-diazatricyclo[3.3.1.13,7]decyl, 2-azatricyclo[3.3.1.13,7]decyl, 2,4,9-triazatricyclo[3.3.1.13,7]decyl, 8-azabicyclo[3.2.1]octyl, 2,5-diazabicyclo[2.2.2]octyl, 2-azabicyclo[2.2.2]octyl, 3-azabicyclo[3.2.1]octyl, 8-azabicyclo[3.2.1]octyl, octahydro-1H-pyrrolo[3,2-b]pyridyl group. This term specifically excludes bicyclic heterocycloalkyl moieties where the rings are joined at a single atom per ring (spiro), as exemplified by the bonding arrangement in a 1-oxa-2-azaspiro[4.5]dec-2-en-3-yl group. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-membered and/or 6-membered heterocycloalkyl groups, such as pyrrolidyl (or pyrrolidinyl), tetrahydrofuryl (or tetrahydrofuranyl), tetrahydrothienyl, dihydrofuryl, oxazolinyl, thiazolinyl or pyrazolinyl, piperidyl (or piperidinyl), piperazinyl, morpholinyl, tetrahydropyranyl, dihydropyranyl, 1,3-dioxanyl, tetrahydro-2H-1,4-thiazinyl, 1,4-dioxanyl, 1,3-oxathianyl, and 1,3-dithianyl.

“Heteroaryl” represents a group or moiety comprising an aromatic monovalent monocyclic or bicyclic radical, containing 5 to 10 ring atoms, including 1 to 4 heteroatoms selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, isobenzofuryl, 2,3-dihydrobenzofuryl, chromenyl, chromanyl, indolizinyl, isoindolyl, indolyl, indazolyl, purinyl, isoquinolyl, quinolyl, phthalazinyl, naphthridinyl, quinzolinyl, benzothiazolyl, benzimidazolyl, tetrahydroquinolinyl, cinnolinyl, pteridinyl, isothiazolyl.

Some of the heteroaryl groups present in the compounds of this invention are 5-6 membered monocyclic heteroaryl groups. Selected 5-membered heteroaryl groups contain one nitrogen, oxygen or sulfur ring heteroatom, and optionally contain 1, 2 or 3 additional nitrogen ring atoms. Selected 6-membered heteroaryl groups contain 1, 2, 3 or 4 nitrogen ring heteroatoms. Selected 5- or 6-membered heteroaryl groups include thienyl, pyrrolyl, imidazolyl, pyrazolyl, furyl, isothiazolyl, furazanyl, isoxazolyl, oxazolyl, oxadiazolyl, thiazolyl, triazolyl, and tetrazolyl or pyridyl, pyrazinyl, pyrimidinyl, pyridazinyl, triazinyl, and thiadiazolyl.

The terms “halogen” and “halo” represent chloro, fluoro, bromo or iodo substituents. “Hydroxy” or “hydroxyl” is intended to mean the radical —OH.

The compounds of the invention are only those which are contemplated to be “chemically stable” as will be appreciated by those skilled in the art.

Accordingly, the invention is further directed to a compound according to Formula I, wherein:

A is —((C₁-C₆)alkyl)C(═O)NR^{X}—, —((C₁-C₆)alkyl)SO₂NR^{X}-—((C₁-C₆)alkyl)NR^{X}C(═O)NR^{X}—, or —((C₁-C₆)alkyl)NR^{X}C(═O)—; particularly —((C₁-C₆)alkyl)C(═O)NR^{X}—, —((C₁-C₆)alkyl)SO₂NR^{X}— or —(C₁-C₆)alkyl)NR^{X}C(═O)—;

each R^{X} is independently selected from H, (C₁-C₄)alkyl, or optionally substituted (C₂-C₄)alkyl, where said optionally substituted (C₂-C₄)alkyl is optionally substituted by hydroxyl, cyano, amino, (C₁-C₄)alkoxy, (C₁-C₄)alkyl)NH—, or ((C₁-C₂)alkyl)((C₁-C₂)alkyl)N—;

n is 1-4;

R² and R³ taken together with the atom to which they are connected form an optionally substituted 4, 5, or 6 membered cycloalkyl or heterocycloalkyl group, wherein said heterocycloalkyl group contains 1 heteroatom selected from N, O and S and said optionally substituted cycloalkyl or heterocycloalkyl group is optionally substituted by a substituent selected from (C₁-C₄)alkyl, halo(C₁-C₄)alkyl, halogen, cyano, aryl(C₁-C₂)alkyl-, (C₃-C₆)cycloalkyl(C₁-C₂)alkyl-, —OR^{Ya}, —NR^{Ya}R^{Yb}, —C(═O)OR^{Ya}, —C(═O)NR^{Ya}R^{Yb}, —NR^{Yb}C(═O)R^{Ya}, —SO₂NR^{Ya}R^{Yb}, —NR^{Yb}SO₂R^{Ya}, —OC(═O)NR^{Ya}R^{Yb}, —NR^{Yb}C(═O)OR^{Ya}, —NR^{Yb}C(═O)NR^{Ya}R^{Yb}, where R^{Ya} is selected from H, (C₁-C₄)alkyl and —(C₁-C₄)alkylphenyl and each R^{Yb} is independently selected from H and (C₁-C₄)alkyl;

or R² and R³ are independently selected from H and optionally substituted (C₁-C₄)alkyl, phenyl(C₁-C₂)alkyl-, and (C₃-C₆)cycloalkyl(C₁-C₂)alkyl-,

or R² is (C₁-C₄)alkylamino, ((C₁-C₃)alkyl)((C₁-C₃)alkyl)amino, amino(C₁-C₄)alkyl, (C₁-C₃)alkylamino(C₁-C₄)alkyl, or ((C₁-C₃)alkyl)((C₁-C₃)alkyl)amino(C₁-C₄)alkyl, and R³ is H or (C₁-C₃)alkyl,

or R² is hydroxyl and R³ is H or methyl;

L is 5-6 membered heteroaryl or phenyl group which is substituted by R⁴ and is optionally further substituted by 1 substituent selected from halogen, cyano and (C₁-C₄)alkyl;

R⁴ is H, (C₁-C₄)alkyl, (C₁-C₄)haloalkyl, (C₁-C₄)alkoxy, ((C₁-C₄)alkyl)((C₁-C₄)alkyl)N(C₁-C₄)alkoxy, (C₁-C₄)alkylamino, optionally substituted phenyl, or optionally substituted 5-6 membered heteroaryl;

or a salt, particularly a pharmaceutically acceptable salt, thereof.

The invention is further directed to a compound according to Formula I, wherein:

A is —((C₁-C₄alkyl)C(═O)NR^{X}—, —((C₁-C₄)alkyl)SO₂NR^{X}—, —((C₁-C₄)alkyl)NR^{X}C(═O)NR^{X}— or —((C₁-C₄)alkyl)NR^{X}C(═O)—; particularly —((C₂-C₄)alkyl)C(═O)NR^{X}—, —((C₂-C₄)alkyl)SO₂NR^{X}— or —((C₂-C₄)alkyl)NR^{X}C(═O)—;

n is 1-3;

R² and R³ taken together with the atom to which they are connected form an optionally substituted 4, 5 or 6 membered cycloalkyl or heterocycloalkyl group, wherein said heterocycloalkyl group contains 1 heteroatom selected from N and O and said optionally substituted cycloalkyl or heterocycloalkyl group is optionally substituted by a substituent selected from (C₁-C₄)alkyl, aryl(C₁-C₂)alkyl-, and (C₃-C₆)cycloalkyl(C₁-C₂)alkyl-;

or R² is selected from H and optionally substituted (C₁-C₄)alkyl, phenyl(C₁-C₂)alkyl-, and (C₃-C₆)cycloalkyl(C₁-C₂)alkyl- and R³ is selected from H and methyl,

or R² is (C₁-C₂)alkylamino, ((C₁-C₂)alkyl)((C₁-C₂)alkyl)amino, amino(C₁-C₃)alkyl, (C₁-C₂)alkylamino(C₁-C₃)alkyl, or ((C₁-C₂)alkyl)((C₁-C₂)alkyl)amino(C₁-C₃)alkyl, and R³ is H or (C₁-C₂)alkyl,

or R² is hydroxyl and R³ is H or methyl;

L is thiazolyl, thienyl, triazolyl, oxazolyl, isoxazolyl or phenyl which is substituted by R⁴ and is optionally further substituted by a methyl group;

R⁴ is H, methyl, phenyl, 4-chlorophenyl, 4-fluorophenyl, 3,5-difluorophenyl, 4-cyanophenyl, 4-methoxyphenyl, pyrid-2-yl, pyrid-3-yl, or pyrid-4-yl;

or a salt, particularly a pharmaceutically acceptable salt, thereof.

Specifically, the invention is further directed to a compound according to Formula I, wherein:

A is —CH₂CH₂C(═O)NH—, —CH₂CH₂CH₂C(═O)NH—, —CH₂CH₂CH₂SO₂NH—, —CH₂CH₂C(CH₃)₂C(═O)NH—, —CH₂CH₂C(CH₃)₂SO₂NH— or —CH₂CH₂CH₂CH₂C(═O)NH—;

R^{X} is H;

n is 1;

R² and R³ taken together with the atom to which they are connected form a tetrahydropyranyl group or R² and R³ are methyl, or R² is —CH₂CH₂N(CH₃)₂ and R³ is H, or R² is hydroxyl and R³ is methyl;

L is thiazolyl or oxazolyl substituted only by R⁴, where R⁴ is phenyl, 4-chlorophenyl, or 4-fluorophenyl;

or a salt, particularly a pharmaceutically acceptable salt, thereof.

More specifically, the invention is further directed to a compound according to Formula I, wherein:

A is —CH₂CH₂CH₂C(═O)NH—, —CH₂CH₂C(CH₃)₂C(═O)NH—, —CH₂CH₂CH₂SO₂NH— or —CH₂CH₂C(CH₃)₂SO₂NH—;

n is 1;

R² and R³ taken together with the atom to which they are connected form an N-methyl-piperidinyl group or R² and R³ are methyl, or R² and R³ are H, or R² is —CH₂CH₂N(CH₃)₂ and R³ is H, or R² is hydroxyl and R³ is H or methyl;

L is thiazolyl or oxazolyl substituted only by R⁴, where R⁴ is phenyl, 4-chlorophenyl, or 4-fluorophenyl;

or a salt, particularly a pharmaceutically acceptable salt, thereof.

In another embodiment, the invention is directed to a compound of Formula (I-a):

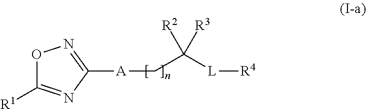

wherein:

R¹ is —CF₃;

n is 0-4;

A is —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —((C₁-C₆)alkyl)SO₂—, —SO₂NR^{X}—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—;

when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,

when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then A is —C(═O)NR^{X}—, —((C₁-C₆)alkyl)C(═O)NR^{X}—, —((C₁-C₆)alkyl)NR^{X}C(═O)NR^{X}, —SO₂NR^{X}—, —((C₁-C₆)alkyl)SO₂NR^{X}—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, —(C₁-C₄)alkyl-, or —((C₁-C₆)alkyl)NR^{X}—, and

when n is 1-4, R² is selected from amino, hydroxyl, (C₁-C₄)alkoxy, and R³ is selected from H and optionally substituted (C₁-C₄)alkyl, aryl(C₁-C₄)alkyl-, and (C₃-C₇)cycloalkyl(C₁-C₄)alkyl-,

wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, halogen, NR^{A}R^{A}, —((C₁-C₄)alkyl)NR^{A}R^{A}, (C₁-C₄)alkoxy, hydroxyl, cyano, halo(C₁-C₄)alkyl, and halo(C₁-C₄)alkoxy;

or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, —NR^{Y}C(═O)NR^{Y}R^{Y}; and

L is 5-6 membered heteroaryl or phenyl which is substituted by R⁴ and is optionally further substituted,

wherein when L is further substituted, L is substituted by 1 or 2 substituents independently selected from halogen, cyano and (C₁-C₄)alkyl;

R⁴ is H, (C₁-C₄)alkyl, halo(C₁-C₄)alkyl, (C₁-C₄)alkoxy, ((C₁-C₄)alkyl)((C₁-C₄)alkyl)N(C₁-C₄)alkoxy, (C₁-C₄)haloalkyl, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,

wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, halo(C₁-C₄)alkoxy, hydroxyl, —NR^{A}R^{A} and —((C₁-C₄)alkyl)NR^{A}R^{A};

or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from halogen, (C₁-C₄)alkyl, cyano, halo(C₁-C₄)alkoxy, (C₁-C₄)alkoxy, and halo(C₁-C₄)alkyl;

wherein each R^{A} is independently selected from H and (C₁-C₄)alkyl;

each R^{X} is independently selected from H, (C₁-C₆)alkyl, or optionally substituted (C₂-C₆)alkyl, where said optionally substituted (C₂-C₆)alkyl is optionally substituted by hydroxyl, cyano, amino, (C₁-C₄)alkoxy, ((C₁-C₄)alkyl)NH—, or ((C₁-C₄)alkyl)((C₁-C₄)alkyl)N—; and

each R^{Y} is independently selected from H, (C₁-C₄)alkyl, phenyl, and —(C₁-C₄)alkylphenyl;

or a salt, particularly a pharmaceutically acceptable salt, thereof.

As used herein, the term “compound(s) of the invention” means a compound of formula (I) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means unsubstituted groups or rings (e.g., cycloalkyl, heterocycle, and heteroaryl rings) and groups or rings substituted with one or more specified substituents.

The compounds according to Formula I may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass all individual stereoisomers and all mixtures thereof. Thus, compounds according to Formula I containing one or more chiral centers may be used as racemic mixtures, scalemic mixtures, or as diaseteromerically or enantiomerically pure materials.

Individual stereoisomers of a compound according to Formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of Formula I are preferably pharmaceutically acceptable salts. Suitable pharmaceutically acceptable salts include those described by Berge, Bighley and Monkhouse, J. Pharm. Sci (1977) 66, pp 1-19. Salts encompassed within the term “pharmaceutically acceptable salts” refer to non-toxic salts of the compounds of this invention.

Typically, a salt may be readily prepared by using a desired acid or base as appropriate. The salt may precipitate from solution and be collected by filtration or may be recovered by evaporation of the solvent.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like, or with a pyranosidyl acid, such as glucuronic acid or galacturonic acid, or with an alpha-hydroxy acid, such as citric acid or tartaric acid, or with an amino acid, such as aspartic acid or glutamic acid, or with an aromatic acid, such as benzoic acid or cinnamic acid, or with a sulfonic acid, such as p-toluenesulfonic acid, methanesulfonic acid, ethanesulfonic acid or the like.

Suitable addition salts are formed from acids which form non-toxic salts and examples include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrabamine, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, di nitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as N-methyl-D-glucamine, diethylamine, isopropylamine, trimethylamine, ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Compounds of the invention having both a basic and acidic moiety may be in the form of zwitterions, acid-addition salt of the basic moiety or base salts of the acidic moiety. This invention also provides for the conversion of one pharmaceutically acceptable salt of a compound of this invention, e.g., a hydrochloride salt, into another pharmaceutically acceptable salt of a compound of this invention, e.g., a sodium salt.

For solvates of the compounds of Formula I, or salts thereof, which are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, e.g., ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The compounds of Formula I may be obtained by using synthetic procedures illustrated in the Schemes below or by drawing on the knowledge of a skilled organic chemist. The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of Formula I, they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

Specific compounds of this invention include the compound of Example 1, N-[4-(4-phenyl-thiazol-2-yl)-tetrahydro-pyran-4-ylmethyl]-4-(5-trifluoromethyl-[1,2,4]oxadiazol-3-yl)-butyramide.

Compounds that may be prepared using the methods described herein include:
N-((4-(4-phenylthiazol-2-yl)tetrahydro-2H-pyran-4-yl)methyl)-4-(5-(trifluoromethyl)-1,2,4-oxadiazol-3-yl)butanamide,
4-(5-(difluoromethyl)-1,2,4-oxadiazol-3-yl)-N-(2-(2-(4-fluorophenyl)oxazol-4-yl)-2-methylpropyl)butanamide,
N-(2-(2-(4-fluorophenyl)oxazol-4-yl)-2-methylpropyl)-4-(5-(trifluoromethyl)-1,2,4-oxadiazol-3-yl)butanamide,
N-(4-(dimethylamino)-2-(2-(4-fluorophenyl)oxazol-4-yl)butyl)-4-(5-(trifluoromethyl)-1,2,4-oxadiazol-3-yl)butanamide,
N-((4-(2-(4-fluorophenyl)oxazol-4-yl)-1-methylpiperidin-4-yl)methyl)-4-(5-(trifluoromethyl)-1,2,4-oxadiazol-3-yl)butanamide,
N-(2-(2-(4-fluorophenyl)oxazol-4-yl)-2-methylpropyl)-2,2-dimethyl-4-(5-(trifluoromethyl)-1,2,4-oxadiazol-3-yl)butanamide,
N-(2-(2-(4-fluorophenyl)oxazol-4-yl)ethyl)-4-(5-(trifluoromethyl)-1,2,4-oxadiazol-3-yl)butanamide,
N-((4-(2-(4-chlorophenyl)thiazol-4-yl)-1-methylpiperidin-4-yl)methyl)-4-(5-(trifluoromethyl)-1,2,4-oxadiazol-3-yl)butanamide,
N-(2-(2-(4-fluorophenyl)oxazol-4-yl)-2-hydroxyethyl)-4-(5-(trifluoromethyl)-1,2,4-oxadiazol-3-yl)butanamide,
4-(5-(difluoromethyl)-1,2,4-oxadiazol-3-yl)-N-(4-(dimethylamino)-2-(2-(4-fluorophenyl)oxazol-4-yl)butyl)butanamide,
N-(2-(2-(4-fluorophenyl)oxazol-4-yl)-2-methylpropyl)-3-(5-(trifluoromethyl)-1,2,4-oxadiazol-3-yl)propane-1-sulfonamide,
N-(3-(2-(4-fluorophenyl)oxazol-4-yl)-3-hydroxypropyl)-3-(5-(trifluoromethyl)-1,2,4-oxadiazol-3-yl)propanamide,
N-(2-(2-(4-fluorophenyl)oxazol-4-yl)-2-methylpropyl)-4-(5-(2,2,2-trifluoroacetyl)thiophen-2-yl)butanamide,
N-(2-(2-(4-fluorophenyl)oxazol-4-yl)ethyl)-4-(5-(2,2,2-trifluoroacetyl)thiophen-2-yl)butanamide,
N-(2-(3-(4-fluorophenyl)-1H-1,2,4-triazol-5-yl)-2-methylpropyl)-4-(5-(2,2,2-trifluoroacetyl)thiophen-2-yl)butanamide,
3-(2-(4-fluorophenyl)oxazol-4-yl)-N-(3-(5-(trifluoromethyl)-1,2,4-oxadiazol-3-yl)propyl)propanamide,
N-(2-(3-(4-fluorophenyl)-1H-1,2,4-triazol-5-yl)ethyl)-4-(5-(2,2,2-trifluoroacetyl)thiophen-2-yl)butanamide,
N-(4-(dimethylamino)-2-(2-(4-fluorophenyl)oxazol-4-yl)butyl)-4-(5-(2,2,2-trifluoroacetyl)thiophen-2-yl)butanamide,
N-((4-(2-(4-fluorophenyl)oxazol-4-yl)-1-methylpiperidin-4-yl)methyl)-4-(5-(2,2,2-trifluoroacetyl)thiophen-2-yl)butanamide,

and a salt, particularly a pharmaceutically acceptable salt, thereof.

Particular compounds of this invention are:
N-(4-(dimethylamino)-2-(2-(4-fluorophenyl)oxazol-4-yl)butyl)-4-(5-(trifluoromethyl)-1,2,4-oxadiazol-3-yl)butanamide,
N-((4-(2-(4-fluorophenyl)oxazol-4-yl)-1-methylpiperidin-4-yl)methyl)-4-(5-(trifluoromethyl)-1,2,4-oxadiazol-3-yl)butanamide,
N-(2-(2-(4-fluorophenyl)oxazol-4-yl)-2-methylpropyl)-2,2-dimethyl-4-(5-(trifluoromethyl)-1,2,4-oxadiazol-3-yl)butanamide,
N-(2-(2-(4-fluorophenyl)oxazol-4-yl)-2-methylpropyl)-4-(5-(trifluoromethyl)-1,2,4-oxadiazol-3-yl)butanamide,
N-(2-(3-(4-fluorophenyl)-1H-1,2,4-triazol-5-yl)ethyl)-4-(5-(2,2,2-trifluoroacetyl)thiophen-2-yl)butanamide,
N-(4-(dimethylamino)-2-(2-(4-fluorophenyl)oxazol-4-yl)butyl)-4-(5-(2,2,2-trifluoroacetyl)thiophen-2-yl)butanamide,
N-(2-(2-(4-fluorophenyl)oxazol-4-yl)-2-hydroxyethyl)-4-(5-(trifluoromethyl)-1,2,4-oxadiazol-3-yl)butanamide,

and a salt, particularly a pharmaceutically acceptable salt, thereof.

Compound names were generated using the software naming program ChemDraw 11.0 available from CambridgeSoft Corporation., 100 CambridgePark Drive, Cambridge, Mass. 02140, USA (http://www.cambridgesoft.com).

The compounds of Formula I can be prepared according to the methods outlined below.

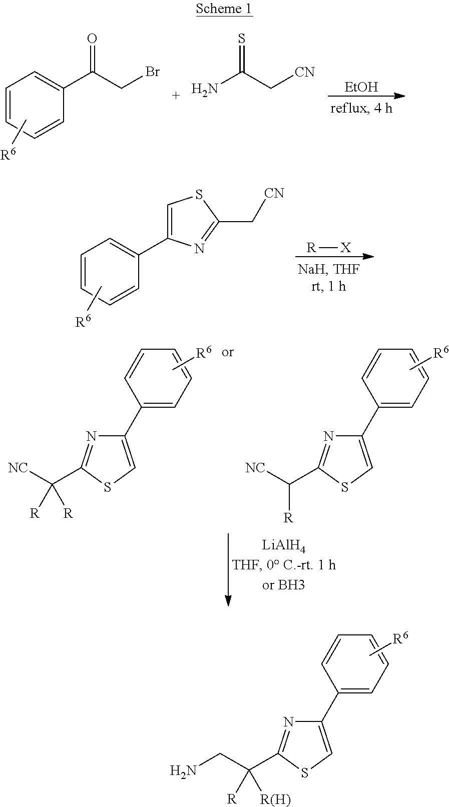

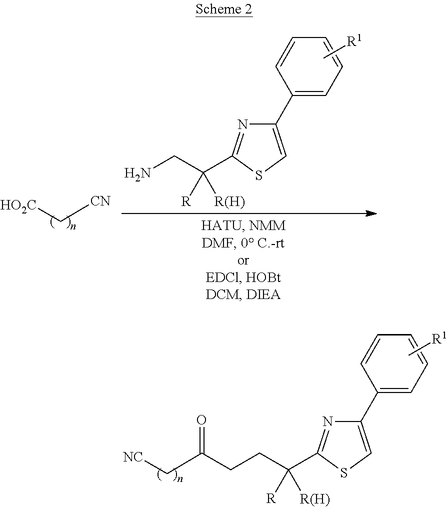

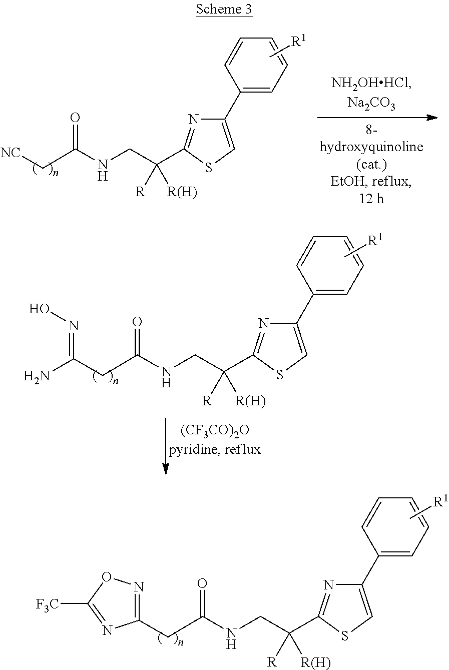

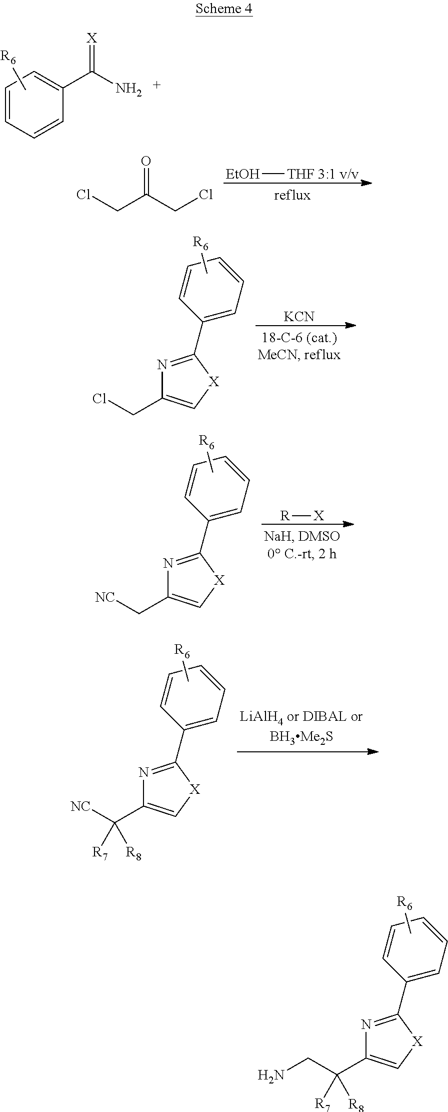

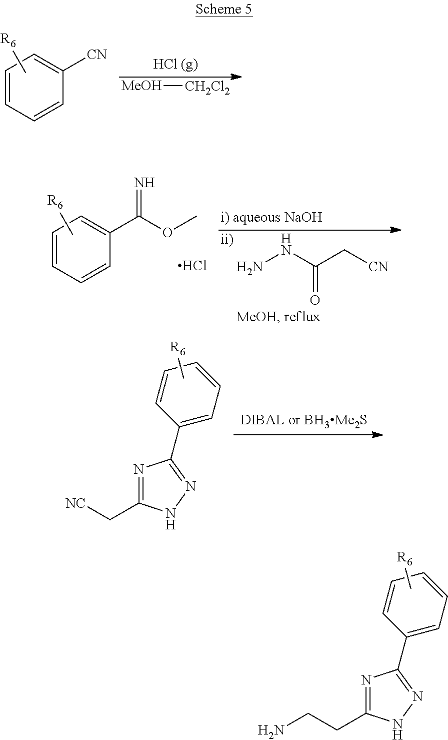

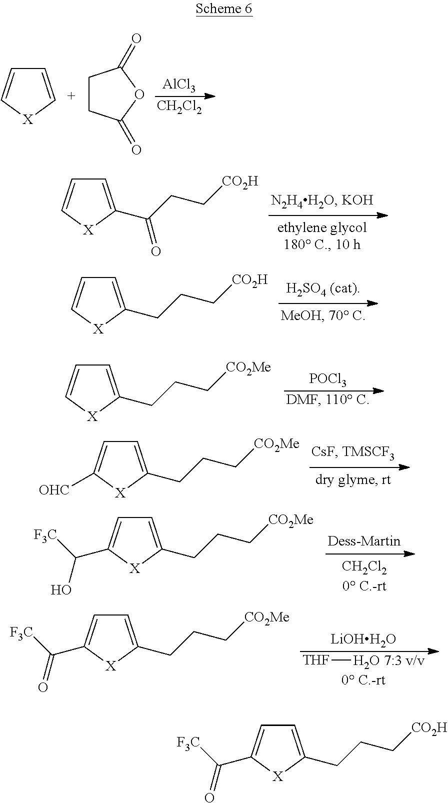

The invention also includes various deuterated forms of the compounds of Formula I. Each available hydrogen atom attached to a carbon atom may be independently replaced with a deuterium atom. A person of ordinary skill in the art will know how to synthesize deuterated forms of the compounds of Formula I. For example, deuterated alkyl groups (e.g., N-(deutero-methyl) amines) may be prepared by conventional techniques (see for example: methyl-d₃-amine available from Aldrich Chemical Co., Milwaukee, Wis., Cat. No. 489, 689-2). Employing such compounds will allow for the preparation of compounds of Formula I in which various hydrogen atoms of the N-methyl groups are replaced with a deuterium atom.

The present invention is directed to a method of inhibiting an HDAC which comprises contacting the acetylase with a compound of Formula I or a salt thereof, particularly a pharmaceutically acceptable salt thereof. This invention is also directed to a method of treatment of an HDAC-mediated disease or disorder comprising administering a therapeutically effective amount of the compound of Formula I or a salt thereof, particularly a pharmaceutically acceptable salt thereof, to a patient, specifically a human, in need thereof. As used herein, “patient” refers to a mammal, specifically, a human. A therapeutically “effective amount” is intended to mean that amount of a compound that, when administered to a patient in need of such treatment, is sufficient to effect treatment, as defined herein. Thus, e.g., a therapeutically effective amount of a compound of Formula I, or a pharmaceutically acceptable salt thereof, is a quantity of an inventive agent that, when administered to a human in need thereof, is sufficient to inhibit the activity of HDAC such that a disease condition which is mediated by that activity is reduced, alleviated or prevented. The amount of a given compound that will correspond to such an amount will vary depending upon factors such as the particular compound (e.g., the potency (pXC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient, where the disease condition is caused or mediated by HDAC. The methods of treatment for mitigation of a disease condition include the use of the compounds in this invention in any conventionally acceptable manner, for example for prevention, retardation, prophylaxis, therapy or cure of a disease.

In one embodiment, this invention is directed to a method of treating, ameliorating, or preventing an autoimmune disorder, an immunological disease, an inflammatory disorder, transplant/graft rejection (e.g., allograft), lymphopenia, or graft-versus-host disease (GvHD) in a patient, specifically in a human, comprising administering to the patient a compound of this invention, in an amount sufficient to increase the level and/or activity of a Treg cell or a population of Treg cells in the patient, thereby treating, ameliorating, or preventing the autoimmune disorder, inflammatory disorder, transplant/graft rejection, lymphopenia, or GvHD in the patient.

Additional examples of diseases and conditions that may be treated by the compounds of this invention include but not limited to coronary artery disease, allergies and allergic reactions, and sepsis/toxic shock.

Exemplary autoimmune disorders include, but are not limited to, multiple sclerosis, juvenile idiopathic arthritis, psoriatic arthritis, hepatitis C virus-associated mixed cryoglobulinemia, polymyositis, dermatomyositis, polyglandular syndrome type II, autoimmune liver disease, Kawasaki disease, myasthenia gravis, immunodysregulation polyendocrinopathy enteropathy X-linked syndrome (IPEX (syndrome)), type I diabetes, psoriasis, hypothyroidism, hemolytic anemia, autoimmune polyendocrinopathy-candidiasis-ectodermal dystrophy (APECED), thrombocytopenia, spondyloarthritis, Sjogren's syndrome, rheumatoid arthritis, inflammatory bowel disease (IBD), Crohn's disease, ulcerative colitis, eczema, gastritis, or thyroiditis. As part of a nonlimiting list, the inflammatory disorder can be contact hypersensitivity, atopic dermatitis or Still disease.

Additional examples of autoimmune diseases include but are not limited to autoimmune diseases include osteoarthritis, systemic sclerosis, sarcoidosis, insulin dependent diabetes mellitus (IDDM, type II diabetes), reactive arthritis, scleroderma, vasculitis, Wegener's granulomatosis, Hashimoto's disease, scleroderma, oophoritis, Lupus (SLE), Grave's disease, asthma, cryoglobulinemia, primary biliary sclerosis, pemphigus vulgaris, hemolytic anemia and pernicious anemia.

Examples of transplant/graft rejection (e.g., allograft), lymphopenia, or graft-versus-host disease (GvHD) are those arising from cell, tissue and organ transplantation procedures, such as therapeutic cell transplants such as stem cells, muscle cells such as cardiac cells, islet cells, liver cells, bone marrow transplants, skin grafts, bone grafts, lung transplants, kidney transplants, liver transplants, and heart transplants.

Other examples of diseases and conditions that may be treated by the compounds of this invention include but are not limited to cystic fibrosis, osteoporosis, obesity, epilepsy, depression, thalassemia, sickle cell anemia, amyotrophic lateral sclerosis (ALS) and hyperalgesia, cardiac disease (e.g., stroke, hypertension, atherothrombotic diseases, artherosclerosis or limitation of infarct size in acute coronary syndrome), diseases or disorders involving muscular atrophy, gentamicin-induced hearing loss, drug resistance (e.g., drug resistance in osteosarcoma and colon cancer cells), infectious diseases, and immune deficiency/immunocompromised patients. Examples of infectious diseases relate to various pathogen infections such as viral, fungal, bacterial, mycoplasm, and infections by unicellular and multicellular eukaryotic organisms. Common human pathogens include but are not limited to HIV, HSV, HPV, Hepatitis A, B and C viruses, influenza, denge, zostrella, rubella, RSV, rotavirus, gram positive, gram negative, streptococcus, tetanus, staphalococcus, tuberculosis, listeria, and malaria.

In another embodiment, this invention is directed to inhibitors of HDAC and their use to stop or reduce the growth of neoplastic cells, e.g., cancer cells and tumor cells.

The growth of cancer cells and/or tumor cells that are found in the following cancer types may be reduced by treatment with a compound of this invention: carcinoma (e.g., adenocarcinoma), heptaocellular carcinoma, sarcoma, myeloma (e.g., multiple myeloma), treating bone disease in multiple myeloma, leukemia, childhood acute lymphoblastic leukemia and lymphoma (e.g., cutaneous cell lymphoma), and mixed types of cancers, such as adenosquamous carcinoma, mixed mesodermal tumor, carcinosarcoma, and teratocarcinoma.

In one aspect of the invention, breast or prostate cancers or tumors are treated using the HDAC inhibitors of this invention.

Other cancers that may be treated using the compounds of this invention include, but are not limited to, bladder cancer, lung cancer, colon cancer, rectal cancer, endometrial cancer, ovarian cancer; head and neck cancer, and melanoma.

The inhibitors of the invention may be employed alone or in combination with standard anti-cancer regimens for neoplastic cell, e.g., tumor and cancer, treatments.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Treatment of HDAC-mediated disease conditions may be achieved using the compounds of this invention as a monotherapy, or in dual or multiple combination therapy, such as in combination with other agents, for example, in combination with one or more of the following agents: DNA methyltransferase inhibitors, acetyl transferase enhancers, proteasome or HSP90 inhibitors, and one or more immunosuppressants that do not activate the T suppressor cells including but are not limited to corticosteroids, rapamycin, Azathioprine, Mycophenolate, Cyclosporine, Mercaptopurine (6-MP), basiliximab, daclizumab, sirolimus, tacrolimus, Muromonab-CD3, cyclophosphamide, and methotrexate, which are administered in effective amounts as is known in the art.

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The compounds of the invention and the pharmaceutically-acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition.

For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

In the following experimental descriptions, the following abbreviations may be used:

| Abbreviation | Meaning |
|---|---|
| AcOH | acetic acid |
| aq | aqueous |
| brine | saturated aqueous NaCl |
| CH₂Cl₂ | methylene chloride |
| CH₃CN or MeCN | acetonitrile |
| CH₃NH₂ | methylamine |
| d | day |
| DMF | N,N-dimethylformamide |
| DMSO | dimethylsulfoxide |
| equiv | equivalents |
| Et | ethyl |
| Et₃N | triethylamine |
| Et₂O | diethyl ether |
| EtOAc | ethyl acetate |
| h, hr | hour |
| HCl | hydrochloric acid |
| i-Pr₂NEt | N′,N′-diisopropylethylamine |
| KOt-Bu | potassium tert-butoxide |
| LCMS | liquid chromatography-mass spectroscopy |
| Me | methyl |
| MeOH or CH₃OH | methanol |
| MgSO₄ | magnesium sulfate |
| min | minute |
| MS | mass spectrum |
| μw | microwave |
| NaBH₄ | sodium borohydride |
| Na₂CO₃ | sodium carbonate |
| NaHCO₃ | sodium bicarbonate |
| NaOH | sodium hydroxide |
| Na₂SO₄ | sodium sulfate |
| NH₄Cl | ammonium chloride |
| NiCl₂•6H₂O | nickel (II) chloride hexahydrate |
| NMP | N-methyl-2-pyrrolidone |
| Ph | phenyl |
| rt | room temperature |
| satd | saturated |
| SCX | strong cation exchange |
| SPE | solid phase extraction |
| TFA | trifluoroacetic acid |
| THF | tetrahydrofuran |
| t_{R} | retention time |

### Example 1

### Step 1: 4-Cyanobutyric acid

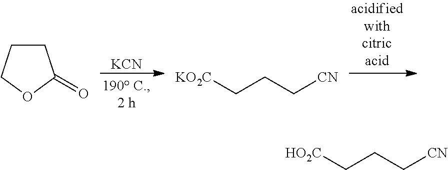

A mixture of γ-butyrolactone (5 g, 0.058 mol) and KCN (4.1 g, 0.064 mol) was heated to 190° C. for 2 h. The resulting reaction mixture was slowly cooled to room temperature and acidified to pH ˜3-4 using citric acid solution. EtOAc was added to the mixture and the two layers were separated after stirring for 30 minutes. The organic layer was collected and washed with water and brine, and dried over anhydrous sodium sulfate. The solvent was evaporated under reduced pressure to get the pure 4-cyanobutyric acid (2.4 g, yield 36%) as a brown liquid. ¹H NMR (300 MHz, DMSO-d₆) δ 12.34 (br s, 1H), 2.50 (t, J=7.2 Hz, 2H), 2.31 (t, J=7.2 Hz, 2H), 1.81-1.71 (m, 2H).

### Step 2: 2-(4-Phenylthiazol-2-yl)acetonitrile

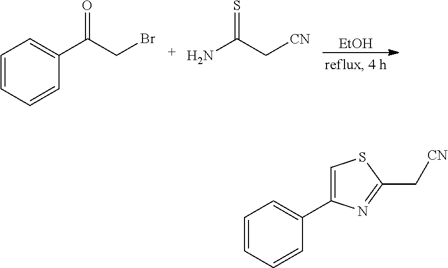

A mixture of 2-bromoacetophenone (2 g, 10 mmol) and 2-cyanothioacetamide (1 g, 10 mmol) in EtOH (25 mL) was heated to 80° C. for 4 h. The reaction mixture was cooled to room temperature and poured into an aqueous ammonia solution (final pH was >7).

The mixture was then extracted with EtOAc and the organic layer was washed with H₂O and brine. Solvent was removed under reduced pressure and the crude product was purified by flash column chromatography (silica gel 230-400 mesh, eluent 8% EtOAc in petroleum ether) to afford 2-(4-phenylthiazol-2-yl)acetonitrile (1.5 g, yield 75%) as a yellow solid: ¹H NMR (300 MHz, CDCl₃) δ 7.88-7.91 (m, 2H), 7.49 (s, 1H), 7.27-7.48 (m, 3H), 4.19 (s, 2H). MS (ESI) m/z: Calculated for C₁₁H₈N₂S: 200.04; found: 201.2 (M+H)⁺.

### Step 3: 4-(4-Phenylthiazol-2-yl)tetrahydro-2H-pyran-4-carbonitrile

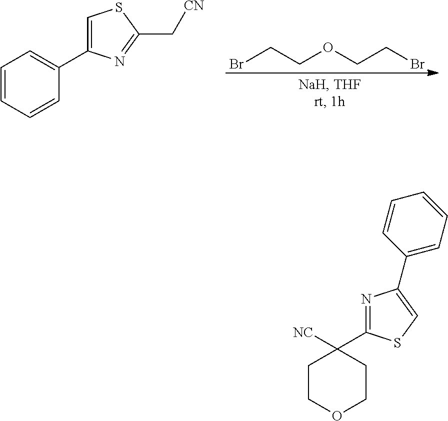

A solution of 2-(4-phenylthiazol-2-yl)acetonitrile (0.84 g, 4.19 mmol) in THF (25 mL) was cooled to 0° C. NaH was added (0.5 g, 60% dispersion in oil) portionwise over 10 min. The resulting mixture was allowed to warm up to room temperature and stirred for 20 min. 2-Bromoethyl ether (1.58 mL, 12.5 mmol) was added dropwise. The reaction mixture was further stirred at room temperature for 1 h and then quenched with saturated NH₄Cl solution. The reaction mixture was diluted with EtOAc and the organic layer was washed with H₂O and brine. The organic layer was dried over anhydrous sodium sulfate and concentrated under reduced pressure. The residue was purified by column chromatography (silica gel 60-120 mesh, eluent 4-8% EtOAc in petroleum ether) to afford 4-(4-phenylthiazol-2-yl)tetrahydro-2H-pyran-4-carbonitrile (0.97 g, yield 85%) as a yellow solid: ¹H NMR (300 MHz, CDCl₃) δ 7.91-7.94 (m, 2H), 7.51 (s, 1H), 7.37-7.48 (m, 3H), 4.07-4.14 (m, 2H), 3.87-3.96 (m, 2H), 2.32-2.43 (m, 4H). MS (ESI) m/z: Calculated for C₁₅H₁₄N₂OS: 270.08; found: 271.2 (M+H)⁺.

### Step 4: (4-(4-Phenylthiazol-2-yl)tetrahydro-2H-pyran-4-yl)methanamine

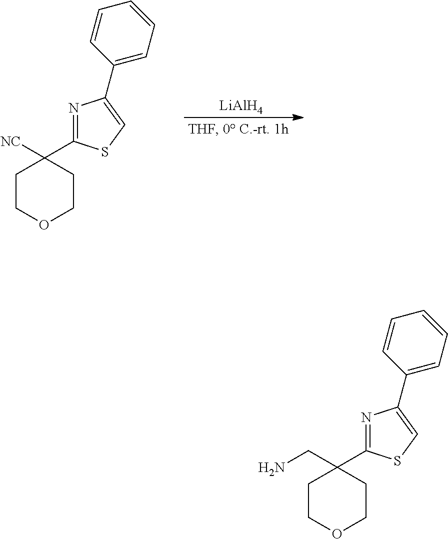

To a suspension of LiAlH₄ (220 mg, 5.9 mmol) in dry THF (10 mL) was added a solution of 4-(4-phenylthiazol-2-yl)tetrahydro-2H-pyran-4-carbonitrile (400 mg, 1.47 mmol) in dry THF (10 mL) at 0° C. The reaction mixture was stirred at room temperature for 1 h and then quenched carefully with water and diluted with EtOAc. The organic layer was washed with brine, dried over anhydrous sodium sulfate and concentrated under reduced pressure. The residue was purified by column chromatography (neutral alumina, eluent 5% MeOH in CHCl₃) to afford (4-(4-phenylthiazol-2-yl)tetrahydro-2H-pyran-4-yl)methanamine (150 mg, yield 37%): ¹H NMR (400 MHz, CDCl₃) δ 7.89-7.91 (m, 2H), 7.48 (s, 1H), 7.33-7.46 (m, 3H), 3.89-3.93 (m, 2H), 3.63-3.69 (m, 2H), 3.03 (s, 2H), 2.30-2.33 (m, 2H), 1.90-1.97 (m, 2H). MS (ESI) m/z: Calculated for C₁₅H₁₈N₂OS: 274.11; found: 275.2 (M+H)⁺.

### Step 5: 4-Cyano-N-((4-(4-phenylthiazol-2-yl)tetrahydro-2H-pyran-4-yl)methyl)butanamide

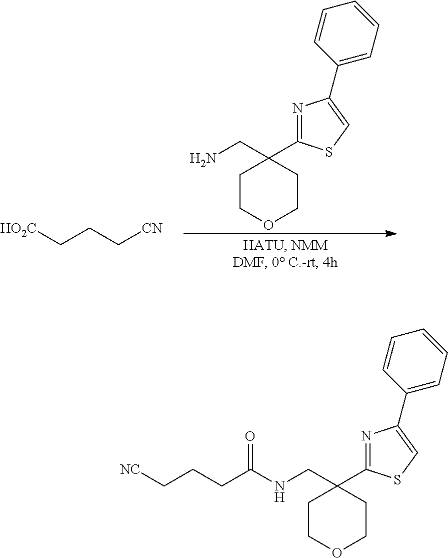

4-Cyanobutyric acid (100 mg, 0.71 mmol) was dissolved in dry DMF (5 mL), and HATU (320 mg, 0.86 mmol) was added, followed by (4-(4-phenylthiazol-2-yl)tetrahydro-2H-pyran-4-yl)methanamine (190 mg, 0.71 mmol) and NMM (0.2 mL, 2.19 mmol) at 0° C. The reaction mixture was slowly warmed to room temperature and stirred for 4 h, then it was diluted with EtOAc. The organic layer was washed with water and brine solution, dried over anhydrous sodium sulfate and concentrated under reduced pressure. The crude product was purified by column chromatography (silica 60-120 mesh, eluant 20-25% EtOAc in petroleum ether) to get pure 4-cyano-N-((4-(4-phenylthiazol-2-yl)tetrahydro-2H-pyran-4-yl)methyl)butanamide (110 mg, yield 46%): ¹H NMR (300 MHz, DMSO-d₆) δ 8.01 (m, 1H), 7.95-7.89 (m, 2H), 7.76 (s, 1H), 7.42-7.37 (m, 2H), 7.33-7.28 (m, 1H), 3.91-3.85 (dt, J=11.8 Hz, 3.9 Hz, 2H), 3.60-3.49 (m, 4H), 2.33-2.24 (m, 6H), 2.00-1.90 (m, 2H), 1.83-1.73 (m, 2H).

### Step 6: 5-Amino-5-(hydroxyimino)-N-((4-(4-phenylthiazol-2-yl)tetrahydro-2H-pyran-4-yl)methyl)pentanamide

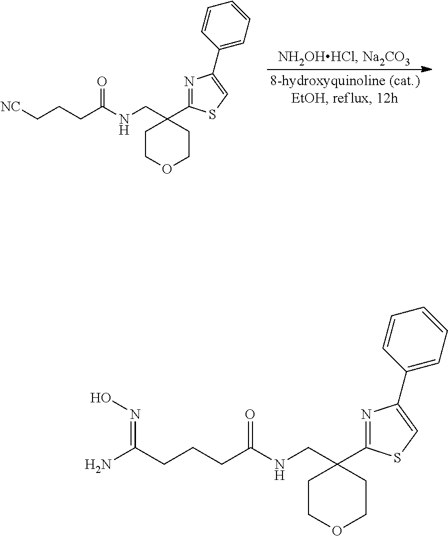

4-Cyano-N-((4-(4-phenylthiazol-2-yl)tetrahydro-2H-pyran-4-yl)methyl)butanamide (110 mg, 0.29 mmol) was dissolved in 5 mL ethanol and 8-hydroxyquinoline (1 mg) was added. To this reaction mixture were added first hydroxylamine hydrochloride (41 mg, 0.59 mmol) in water (0.5 mL) followed by sodium carbonate (50 mg, 0.47 mmol) in water (0.5 mL). The mixture was heated to reflux for 12 h. Solvent was removed under reduced pressure and the crude product 5-amino-5-(hydroxyimino)-N-((4-(4-phenylthiazol-2-yl)tetrahydro-2H-pyran-4-yl)methyl)pentanamide (100 mg, crude) was carried through without further purification. ¹H NMR (300 MHz, DMSO-d₆) δ 8.69 (m, 1H), 8.06 (s, 1H), 7.96-7.93 (m, 2H), 7.84-7.80 (m, 1H), 7.45-7.40 (m, 2H), 7.34-7.32 (m, 1H), 4.12-4.09 (m, 2H), 3.80-3.75 (m, 2H), 3.42-3.38 (m, 4H), 3.15-3.14 (m, 2H), 2.53 (m, 2H), 2.12-2.02 (m, 2H), 1.89-1.84 (m, 2H). MS (ESI) m/z: Calculated for C₂₀H₂₆N₄O₃S: 402.17; found: 403.2 (M+H)⁺.

### Step 7: N-((4-(4-Phenylthiazol-2-yl)tetrahydro-2H-pyran-4-yl)methyl)-4-(5-(trifluoromethyl)-1,2,4-oxadiazol-3-yl)butanamide

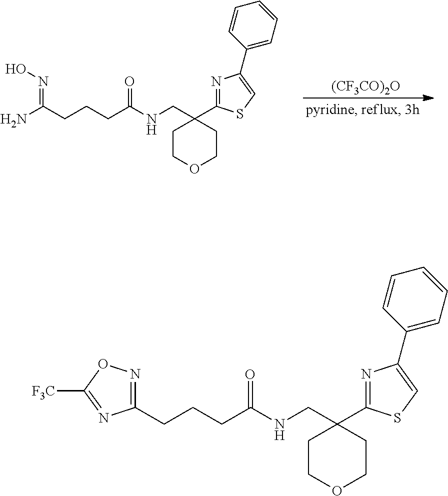

A solution of 5-amino-5-(hydroxyimino)-N-((4-(4-phenylthiazol-2-yl)tetrahydro-2H-pyran-4-yl)methyl)pentanamide (100 mg, 0.24 mmol) in anhydrous pyridine (2 mL) was cooled to 0° C. and trifluoroacetic anhydride (0.1 mL, 0.74 mmol) was added dropwise. The reaction mixture was slowly warmed to room temperature and further refluxed at 110° C. for 3 h. The reaction mixture was diluted with EtOAc and the organic layer was washed with water and brine. Solvent was removed under reduced pressure and the crude product was purified by column chromatography [silica gel 60-120 mesh, eluant: 15% EtOAc in petroleum ether] to afford N-((4-(4-phenylthiazol-2-yl)tetrahydro-2H-pyran-4-yl)methyl)-4-(5-(trifluoromethyl)-1,2,4-oxadiazol-3-yl)butanamide (30 mg, yield 28%). ¹H NMR (400 MHz, MeOD) δ 7.94-7.91 (dd, J=8.3 Hz, 1.0 Hz, 2H), 7.78 (s, 1H), 7.39-7.36 (m, 2H), 7.31-7.27 (m, 1H), 3.92-3.87 (dt, J=11.9 Hz, 3.8 Hz, 2H), 3.61-3.52 (m, 4H), 2.76 (t, J=7.5 Hz, 2H), 2.36-2.32 (d, J=13.8 Hz, 2H), 2.27-2.23 (m, 2H), 2.00-1.94 (m, 4H). MS (ESI) m/z: Calculated for C₂₂H₂₃F₃N₄O₃S: 480.14; found: 481.2 (M+H)⁺.

### Example 2

### 4-(Chloromethyl)-2-(4-fluorophenyl)oxazole

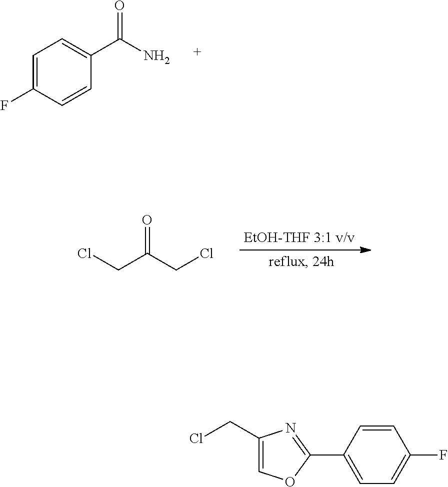

A mixture of 4-fluorobenzamide (2.5 g, 17.9 mmol) and 1,3-dichloroacetone (2.7 g, 21.6 mmol) in EtOH-THF (20 mL-10 mL) was heated to 85° C. for 24 h. The reaction mixture was cooled to room temperature and quenched with 10% NaHCO₃ solution. The organic product was extracted with EtOAc and the organic layer was washed with H₂O and brine, dried over anhydrous sodium sulfate and concentrated under reduced pressure. The crude product was purified by column chromatography (silica gel 60-120 mesh, eluent 6-10% EtOAc in petroleum ether) to afford 4-(chloromethyl)-2-(4-fluorophenyl)oxazole (1.2 g, yield 32%) as white solid. ¹H NMR (300 MHz, CDCl₃) δ 8.07-8.02 (m, 2H), 7.70 (m, 1H), 7.19-7.13 (t, J=8.8 Hz, 2H), 4.58 (m, 2H). MS (ESI) m/z: Calculated for C₁₀H₇FClNO: 211.02; found: 212.0 (M+H)⁺.

### 2-(2-(4-Fluorophenyl)oxazol-4-yl)acetonitrile

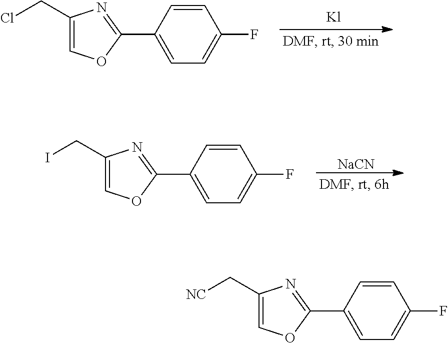

KI (3.14 g, 18.9 mmol) was added to a solution of 4-(chloromethyl)-2-(4-fluorophenyl)oxazole (1.0 g, 4.7 mmol) in dry DMF (20 mL) at room temperature. The reaction mixture was stirred for 30 min, diluted with EtOAc, and washed with water and brine. The organic layer was dried over anhydrous sodium sulfate and concentrated under reduced pressure to give crude 2-(4-fluorophenyl)-4-(iodomethyl)oxazole. The crude product was dissolved in DMF (20 mL) and sodium cyanide (0.46 g, 9.4 mmol) was added to the solution. The reaction mixture was stirred at room temperature for 6 h and quenched with water. The organic product was extracted with EtOAc and the organic layer was washed with H₂O and brine, dried over anhydrous sodium sulfate and concentrated under reduced pressure. The crude product was purified by column chromatography (silica gel 60-120 mesh, eluent 10-15% EtOAc in petroleum ether) to afford 2-(2-(4-fluorophenyl)oxazol-4-yl)acetonitrile (0.75 g, yield 78%) as off-white solid. ¹H NMR (300 MHz, CDCl₃) δ 8.05-8.00 (m, 2H), 7.73 (m, 1H), 7.20-7.14 (t, J=8.6 Hz, 2H), 3.73 (s, 2H). MS (ESI) m/z: Calculated for C₁₁H₇FN₂O: 202.05; found: 203.0 (M+H)⁺.

### 2-(2-(4-Fluorophenyl)oxazol-4-yl)-2-methylpropanenitrile

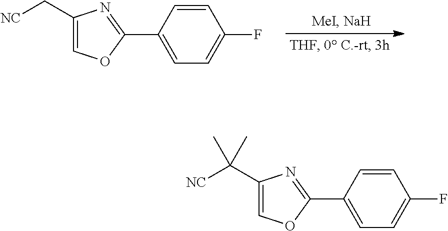

This compound was synthesized from 2-(2-(4-fluorophenyl)oxazol-4-yl)acetonitrile using iodomethane as described in example 1 step 3 (15 g, yield 66%) as white solid. ¹H NMR (300 MHz, CDCl₃) δ 8.07-8.02 (dd, J=8.9 Hz, 5.4 Hz, 2H), 7.66 (s, 1H), 7.19-7.13 (t, J=8.7 Hz, 2H), 1.76 (s, 6H). MS (ESI) m/z: Calculated for C₁₃H₁₁ FN₂O: 230.09; found: 231.2 (M+H)⁺.

### 2-(2-(4-Fluorophenyl)oxazol-4-yl)-2-methylpropan-1-amine

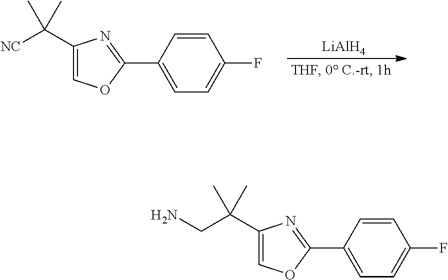

This compound was synthesized from 2-(2-(4-fluorophenyl)oxazol-4-yl)-2-methylpropanenitrile as described in example 1 step 4 (14 g, crude) and it was carried through without further purification. ¹H NMR (300 MHz, MeOD) δ 8.07-8.03 (dd, J=8.9 Hz, 5.4 Hz, 2H), 7.73 (s, 1H), 7.26-7.20 (m, 2H), 2.85 (s, 2H), 1.31 (s, 6H). MS (ESI) m/z: Calculated for C₁₃H₁₅FN₂O: 234.12; found: 235.2 (M+H)⁺.

### 4-Cyano-N-(2-(2-(4-fluorophenyl)oxazol-4-yl)-2-methylpropyl)butanamide

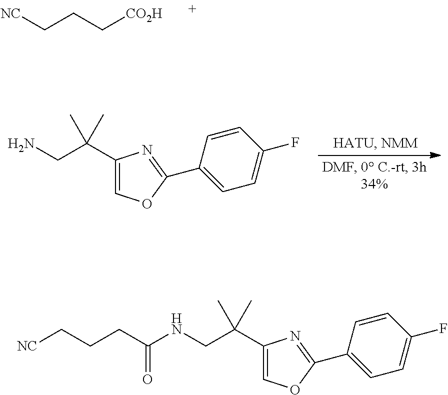

This compound was synthesized from 4-cyanobutanoic acid and 2-(2-(4-fluorophenyl)oxazol-4-yl)-2-methylpropan-1-amine as described in example 1 step 5 (500 mg, yield 34%). ¹H NMR (300 MHz, CDCl₃) δ 8.05-8.00 (m, 2H), 7.44 (s, 1H), 7.20-7.14 (t, J=8.8 Hz, 2H), 6.66-6.64 (m, 1H), 3.46-3.44 (d, J=5.7 Hz, 2H), 2.50-2.45 (m, 2H), 2.43-2.38 (m, 2H), 2.05-2.01 (m, 2H), 1.31 (m, 6H). MS (ESI) m/z: Calculated for C₁₈H₂₀FN₃O₂: 329.15; found: 330.2 (M+H)⁺.

### 5-Amino-N-(2-(2-(4-fluorophenyl)oxazol-4-yl)-2-methylpropyl)-5-(hydroxyimino)pentanamide

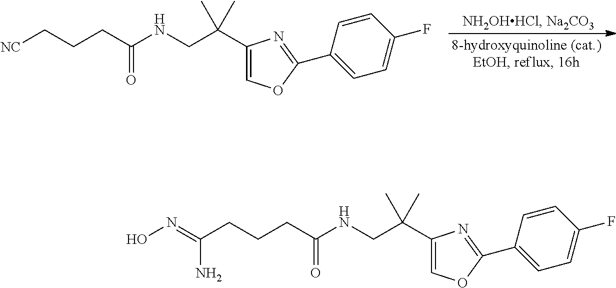

This compound was synthesized from 4-cyano-N-(2-(2-(4-fluorophenyl)oxazol-4-yl)-2-methylpropyl)butanamide as described in example 1 step 6 (250 mg, crude) and it was carried through without further purification. ¹H NMR (400 MHz, DMSO-d₆) δ 8.71 (s, 1H), 8.03-8.00 (m, 2H), 7.92 (s, 1H), 7.63-7.60 (m, 1H), 7.39-7.35 (t, J=8.9 Hz, 2H), 5.32 (br s, 2H), 3.28-3.27 (d, J=6.1 Hz, 2H), 2.10-2.07 (m, 2H), 1.95-1.91 (m, 2H), 1.71-1.67 (m, 2H), 1.19 (s, 6H)

### 4-(5-(Difluoromethyl)-1,2,4-oxadiazol-3-yl)-N-(2-(2-(4-fluorophenyl)oxazol-4-yl)-2-methylpropyl)butanamide

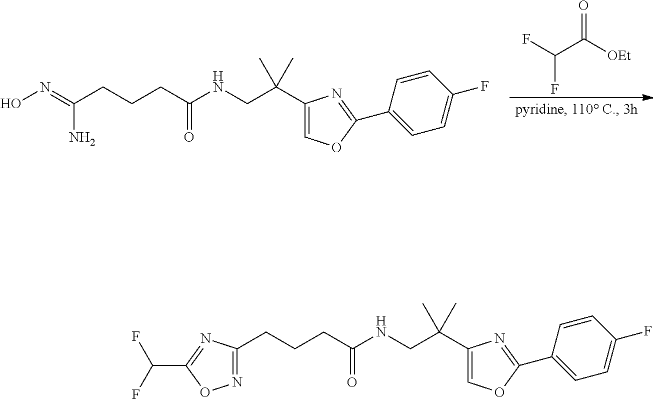

This compound was synthesized from 5-amino-N-(2-(2-(4-fluorophenyl)oxazol-4-yl)-2-methylpropyl)-5-(hydroxyimino)pentanamide using ethyldifluoro acetate as described in example 1 step 7 (45 mg, yield 15%). ¹H NMR (400 MHz, DMSO-d₆) δ 8.01-7.98 (m, 2H), 7.91 (s, 1H), 7.67-7.64 (m, 1H), 7.58-7.33 (m, 3H), 3.28-3.27 (d, J=6.4 Hz, 2H), 2.78-2.74 (t, J=7.3 Hz, 2H), 2.19-2.16 (t, J=7.3 Hz, 2H), 1.92-1.85 (m, 2H), 1.19 (s, 6H). MS (ESI) m/z: Calculated for C₂₀H₂₁F₃N₄O₃: 422.16; found: 423.1 (M+H)⁺.

### Example 3

### N-(2-(2-(4-Fluorophenyl)oxazol-4-yl)-2-methylpropyl)-4-(5-(trifluoromethyl)-1,2,4-oxadiazol-3-yl)butanamide

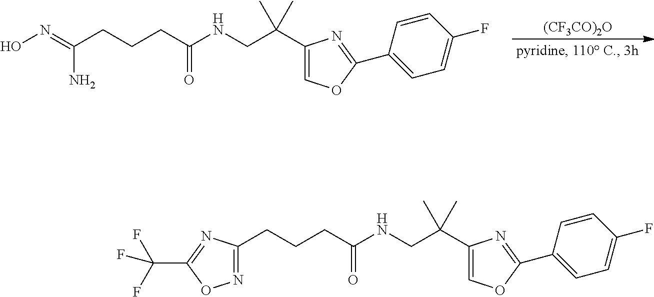

This compound was synthesized from 5-amino-N-(2-(2-(4-fluorophenyl)oxazol-4-yl)-2-methylpropyl)-5-(hydroxyimino)pentanamide as described in example 1 step 7 (60 mg, yield 12%). ¹H NMR (400 MHz, CDCl₃) δ 8.02-7.99 (m, 2H), 7.44 (m, 1H), 7.18-7.13 (t, J=8.7 Hz, 2H), 6.59 (br s, 1H), 3.46-3.45 (d, J=5.9 Hz, 2H), 2.94-2.91 (m, 2H), 2.35-2.31 (m, 2H), 2.21-2.14 (m, 2H), 1.31 (s, 6H). MS (ESI) m/z: Calculated for C₂₀H₂₀F₄N₄O₃: 440.15; found: 441.2 (M+H)⁺.

### Example 4

### 5-Amino-5-(hydroxyimino)pentanoic acid

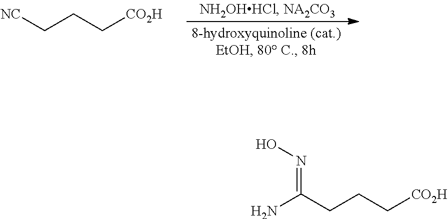

This compound was synthesized from 4-cyanobutyric acid as described for example 1 step 6 (5.3 g, crude) and it was carried through without further purification. ¹H NMR (400 MHz, DMSO-d₆) δ 12.64 (br s, 1H), 10.94 (br s, 1H), 10.39 (br s, 2H), 2.42-2.38 (m, 2H), 2.26-2.23 (m, 2H), 1.86-1.79 (m, 2H). Calculated for C₅H₁₀N₂O₃: 146.07; found: 147.0 (M+H)⁺.

### 4-(5-(Trifluoromethyl)-1,2,4-oxadiazol-3-yl)butanoic acid

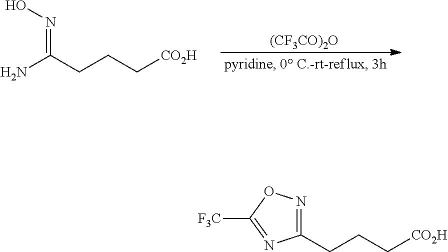

This compound was synthesized from 5-amino-5-(hydroxyimino)pentanoic acid as described for example 1 step 7 (2 g, yield 25%) as light yellow viscous liquid. ¹H NMR (300 MHz, DMSO-d₆) δ 2.90-2.85 (t, J=7.5 Hz, 2H), 2.35-2.30 (t, J=7.3 Hz, 2H), 1.95-1.85 (m, 2H).

### 4-(Dimethylamino)-2-(2-(4-fluorophenyl)oxazol-4-yl)butanenitrile

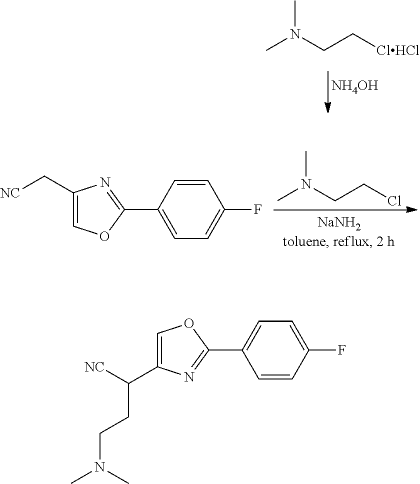

Sodium amide (960 mg, 24.7 mmol) was suspended in toluene (15 mL) and cooled to 0° C. To this suspension was added dropwise a solution of 2-(2-(4-fluorophenyl)oxazol-4-yl)acetonitrile (2.5 g, 12.3 mmol) in toluene (30 mL) while maintaining the temperature at 0° C. The reaction mixture was stirred for 20 min. Separately the bis-(2-chloroethyl)-methyl-amine hydrochloride (2.13 g, 14.8 mmol) was taken in water (4 mL), cooled to 0° C., and basified with aqueous ammonia solution (adjusted to pH of the solution to ˜8). The oily layer was separated out from the aqueous layer and the organic product was extracted with toluene. The toluene layer was dried over sodium hydroxide pellets. The dry toluene solution of the compound bis-(2-chloroethyl)-methyl-amine was added to the reaction mixture at 0° C. The reaction mixture was allowed to warm up to room temperature and further heated to 110° C. for 3 h. The reaction mixture was then cooled to room temperature and diluted with EtOAc. The organic product was extracted with EtOAc and the combined extracts were washed with water and brine, dried over anhydrous sodium sulfate and concentrated under reduced pressure. The crude product was purified by column chromatography (silica gel 60-120 mesh, eluent 5% MeOH in EtOAc followed by 5% MeOH in CH₂Cl₂) to afford 4-(dimethylamino)-2-(2-(4-fluorophenyl)oxazol-4-yl)butanenitrile (1.15 g, yield 35%). ¹H NMR (400 MHz, MeOD) δ 8.10-8.07 (m, 2H), 7.99 (m, 1H), 7.29-7.25 (t, J=8.9 Hz, 2H), 4.24-4.20 (t, J=7.3 Hz, 1H), 2.59-2.46 (m, 2H), 2.29 (s, 6H), 2.23-2.17 (m, 2H). MS (ESI) m/z: Calculated for C₁₅H₁₆FN₃O: 273.13; found: 274.2 (M+H)⁺.

### 3-(2-(4-Fluorophenyl)oxazol-4-yl)-N1,N1-dimethylbutane-1,4-diamine

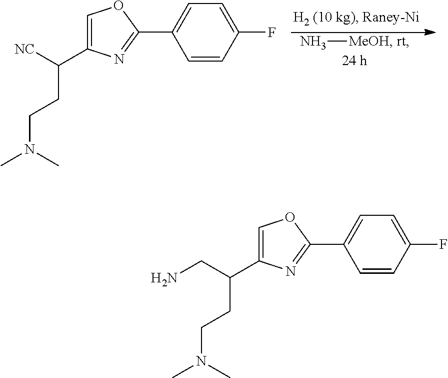

4-(Dimethylamino)-2-(2-(4-fluorophenyl)oxazol-4-yl)butanenitrile (400 mg, 1.46 mmol) was dissolved in saturated solution of ammonia in methanol (40 mL) and Raney-nickel (700 mg) was added to it at room temperature. The resulting mixture was hydrogenated in autoclave under 10 kg/cm² pressure for 24 h. The reaction mixture was filtered through a Celite bed. The filtrate was concentrated under reduced pressure to afford crude 3-(2-(4-fluorophenyl)oxazol-4-yl)-N1N1-dimethylbutane-1,4-diamine (320 mg, crude), which was carried through without further purification. MS (ESI) m/z: Calculated for C₁₅H₂₀FN₃O: 277.16; found: 278.2 (M+H)⁺.

### N-(4-(Dimethylamino)-2-(2-(4-fluorophenyl)oxazol-4-yl)butyl)-4-(5-(trifluoromethyl)-1,2,4-oxadiazol-3-yl)butanamide

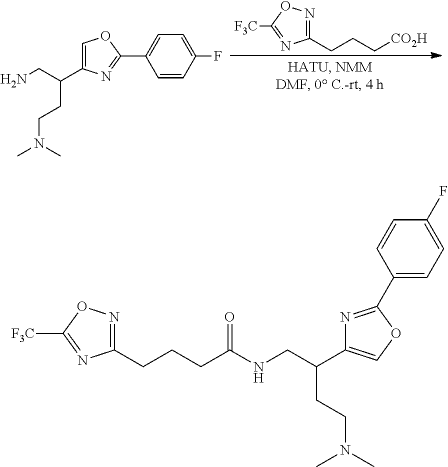

This compound was synthesized from 3-(2-(4-fluorophenyl)oxazol-4-yl)-N1,N1-dimethylbutane-1,4-diamine and 4-(5-(trifluoromethyl)-1,2,4-oxadiazol-3-yl)butanoic acid as described in example 1 step 5 (60 mg, yield 19%). ¹H NMR (400 MHz, MeOD) δ 8.08-8.04 (m, 2H), 7.85 (s, 1H), 7.28-7.23 (t, J=8.8 Hz, 2H), 3.58-3.53 (m, 1H), 3.47-3.42 (m, 1H), 3.17-3.10 (m, 1H), 3.03-2.96 (m, 2H), 2.88-2.84 (t, J=7.5 Hz, 2H), 2.81 (s, 6H), 2.31-2.28 (m, 2H), 2.12-2.02 (m, 4H). MS (ESI) m/z: Calculated for C₂₂H₂₅F₄N₅O₃: 483.19; found: 484.2 (M+H)⁺.

### Example 5

### 4-(2-(4-Fluorophenyl)oxazol-4-yl)-1-methylpiperidine-4-carbonitrile

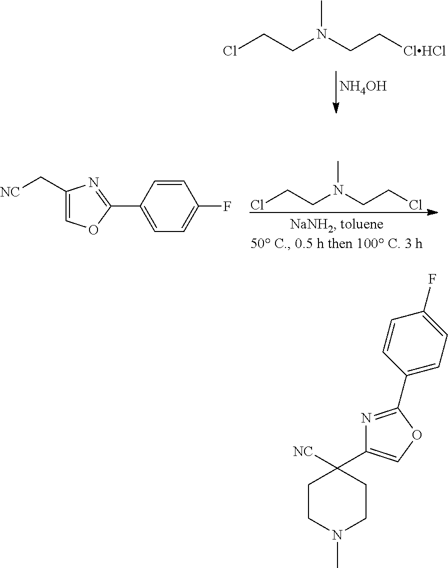

This compound was synthesized from 2-(2-(4-fluorophenyl)oxazol-4-yl)acetonitrile and bis-(2-chloroethyl)-methyl-amine hydrochloride as described in example 4 step 3 (430 mg, yield 30%). ¹H NMR (300 MHz, DMSO-d₆) δ 8.30 (s, 1H), 8.05-8.00 (m, 2H), 7.41-7.35 (m, 2H), 2.83-2.79 (m, 2H), 2.26-2.18 (m, 7H), 2.04-1.94 (m, 2H). MS (ESI) m/z: Calculated for C₁₆H₁₆FN₃O: 285.13; found: 286.2 (M+H)⁺.

### (4-(2-(4-Fluorophenyl)oxazol-4-yl)-1-methylpiperidin-4-yl)methanamine

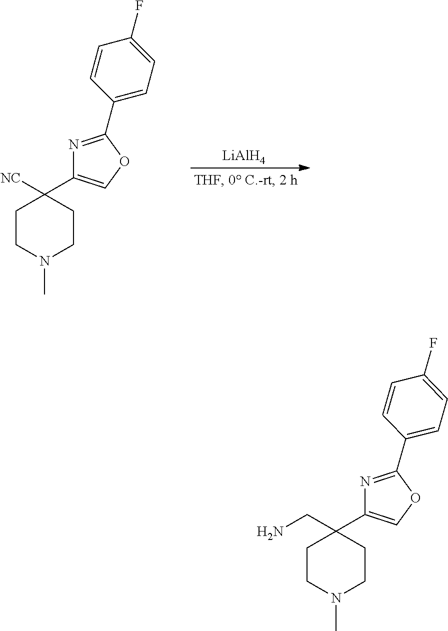

This compound was synthesized from 4-(2-(4-fluorophenyl)oxazol-4-yl)-1-methylpiperidine-4-carbonitrile as described in example 1 step 4 (170 mg, crude) and it was carried through without further purification. MS (ESI) m/z: Calculated for C₁₆H₂₀FN₃O: 289.16; found: 290.2 (M+H)⁺.

### N-((4-(2-(4-Fluorophenyl)oxazol-4-yl)-1-methylpiperidin-4-yl)methyl)-4-(5-(trifluoromethyl)-1,2,4-oxadiazol-3-yl)butanamide

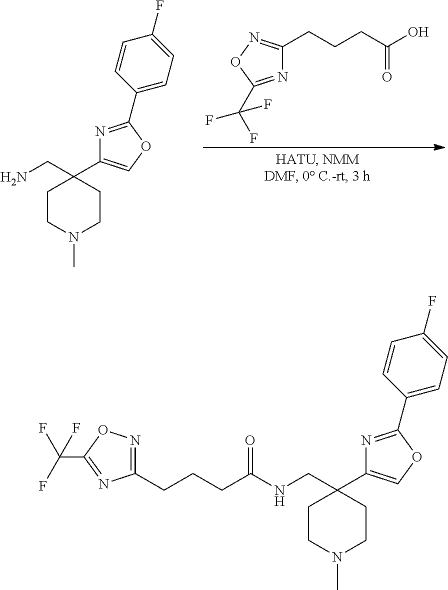

This compound was synthesized from (4-(2-(4-fluorophenyl)oxazol-4-yl)-1-methylpiperidin-4-yl)methanamine and 4-(5-(trifluoromethyl)-1,2,4-oxadiazol-3-yl)butanoic acid as described in example 1 step 5 (25 mg, yield 10%). ¹H NMR (400 MHz, MeOD) δ 8.08-8.04 (m, 2H), 7.81 (s, 1H), 7.25-7.21 (t, J=8.9 Hz, 2H), 3.43 (m, 2H), 2.85-2.81 (t, J=7.5 Hz, 4H), 2.39 (m, 2H), 2.35 (s, 3H), 2.29-2.25 (m, 2H), 2.23-2.19 (m, 2H), 2.06-1.98 (m, 2H), 1.90-1.83 (m, 2H). MS (ESI) m/z: Calculated for C₂₃H₂₅F₄N₅O₃: 495.19; found: 496.2 (M+H)^{+\}.

### Example 6

### 4-Cyano-N-(2-(2-(4-fluorophenyl)oxazol-4-yl)-2-methylpropyl)-2,2-dimethylbutanamide

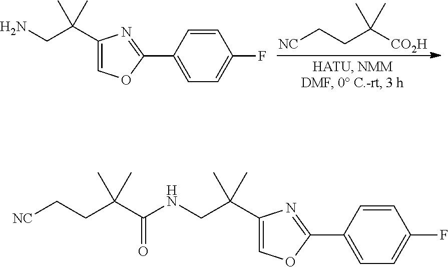

This compound was synthesized from 2-(2-(4-fluorophenyl)oxazol-4-yl)-2-methylpropan-1-amine and 4-cyano-2,2-dimethylbutanoic acid as described in example 1 step 5 (500 mg, yield 40%). ¹H NMR (300 MHz, CDCl₃) δ 8.03-7.98 (m, 2H), 7.45 (s, 1H), 7.20-7.14 (t, J=8.6 Hz, 2H), 3.39-3.37 (d, J=5.5 Hz, 2H), 2.40-2.34 (m, 2H), 1.99-1.93 (m, 2H), 1.32-1.31 (m, 12H). MS (ESI) m/z: Calculated for C₂₀H₂₄FN₃O₂: 357.19; found: 358.1 (M+H)⁺.

### 5-Amino-N-(2-(2-(4-fluorophenyl)oxazol-4-yl)-2-methylpropyl)-5-(hydroxyimino)-2,2-dimethylpentanamide

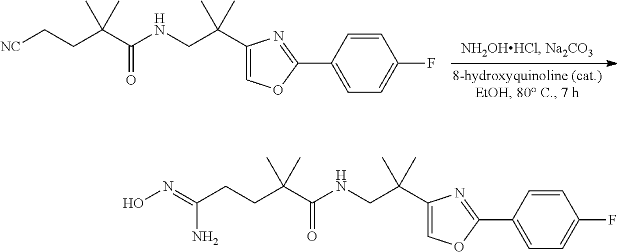

This compound was synthesized from 4-cyano-N-(2-(2-(4-fluorophenyl)oxazol-4-yl)-2-methylpropyl)-2,2-dimethylbutanamide as described in example 1 step 6 (100 mg, crude) and it was carried through without further purification. ¹H NMR (300 MHz, DMSO-d₆) δ 8.68 (br s, 1H), 8.02-7.97 (m, 2H), 7.94 (s, 1H), 7.39-7.33 (m, 2H), 5.26 (br s, 2H), 3.26-3.24 (d, J=4.8 Hz, 2H), 1.88-1.82 (m, 2H), 1.69-1.64 (m, 2H), 1.18 (m, 6H), 1.07 (m, 6H). MS (ESI) m/z: Calculated for C₂₀H₂₇FN₄O₃: 390.21; found: 391.1 (M+H)⁺.

### N-(2-(2-(4-Fluorophenyl)oxazol-4-yl)-2-methylpropyl)-2,2-dimethyl-4-(5-(trifluoromethyl)-1,2,4-oxadiazol-3-yl)butanamide

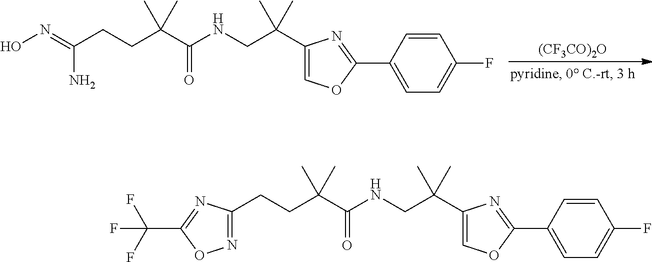

This compound was synthesized from 5-amino-N-(2-(2-(4-fluorophenyl)oxazol-4-yl)-2-methylpropyl)-5-(hydroxyimino)-2,2-dimethylpentanamide as described in example 1 step 7 (33 mg, yield 27%). ¹H NMR (400 MHz, CDCl₃) δ 8.02-7.99 (m, 2H), 7.46 (s, 1H), 7.39-7.36 (m, 1H), 7.19-7.14 (t, J=8.7 Hz, 2H), 3.42-3.41 (d, J=5.3 Hz, 2H), 2.87-2.83 (m, 2H), 2.08-2.04 (m, 2H), 1.34 (m, 12H). MS (ESI) m/z: Calculated for C₂₂H₂₄F₄N₄O₃: 468.18; found: 469.2 (M+H)⁺.

### Example 7

### 2-(2-(4-Fluorophenyl)oxazol-4-yl)ethanamine

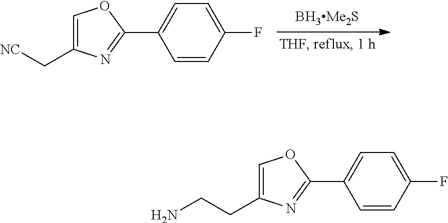

BH₃.Me₂S (˜1.0 mL, 9.9 mmol) was added dropwise to a stirred solution of 2-(2-(4-fluorophenyl)oxazol-4-yl)acetonitrile (500 mg, 2.47 mmol) in dry THF (5 mL) at 0° C. The reaction mixture was stirred at 60° C. for 1 h. The reaction mixture was quenched with MeOH and stirred for 10 min at room temperature. The crude reaction mixture was diluted with EtOAc and organic layer was washed with water and brine, dried over anhydrous sodium sulfate, and concentrated under reduced pressure. The crude product was purified by flash column chromatography (neutral alumina, eluant 3% MeOH in CHCl₃), to get pure 2-(2-(4-fluorophenyl)oxazol-4-yl)ethanamine (190 mg, yield 37%). MS (ESI) m/z: Calculated for C₁₁H₁₁FN₂O: 206.09; found: 206.9 (M+H)⁺.

### N-(2-(2-(4-Fluorophenyl)oxazol-4-yl)ethyl)-4-(5-(trifluoromethyl)-1,2,4-oxadiazol-3-yl)butanamide

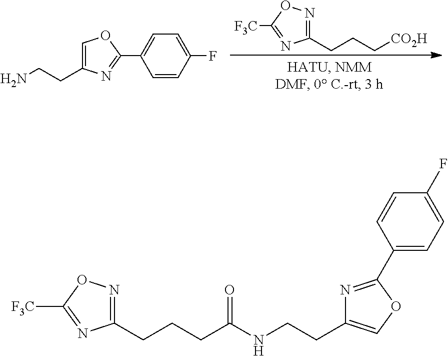

This compound was synthesized from (2-(2-(4-fluorophenyl)oxazol-4-yl)ethanamine and 4-(5-(trifluoromethyl)-1,2,4-oxadiazol-3-yl)butanoic acid as described in example 1 step 5 (60 mg, yield 32%). ¹H NMR (400 MHz, CDCl₃) δ 8.02-7.99 (m, 2H), 7.51 (s, 1H), 7.18-7.13 (t, J=8.8 Hz, 2H), 6.28 (br s, 1H), 3.64-3.60 (m, 2H), 2.94-2.90 (t, J=7.3 Hz, 2H), 2.81-2.77 (t, J=6.7 Hz, 2H), 2.33-2.29 (m, 2H), 2.20-2.12 (m, 2H). MS (ESI) m/z: Calculated for C₁₈H₁₆F₄N₄O₃: 412.12; found: 413.1 (M+H)⁺.

### Example 8

### 4-(Chloromethyl)-2-(4-chlorophenyl)thiazole

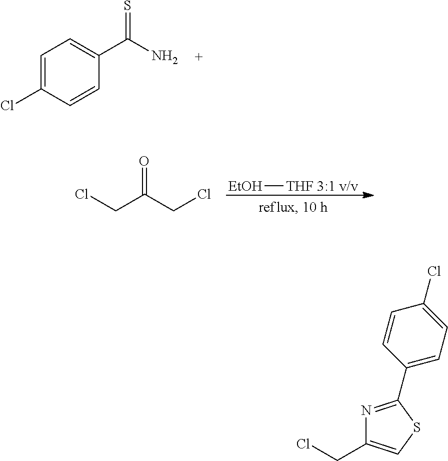

A mixture of 4-chlorothiobenzamide (0.5 g, 2.9 mmol) and 1,3-dichloroacetone (0.4 g, 3.18 mmol) in EtOH-THF (20 mL-10 mL) was heated to 85° C. for 10 h. The reaction mixture was cooled to room temperature and quenched with 10% NaHCO₃ solution. The organic product was extracted with EtOAc and the organic layer was washed with H₂O and brine, and dried over anhydrous sodium sulfate. The solvent was removed under reduced pressure and the crude product was purified by column chromatography (silica gel 60-120 mesh, eluent 3-5% EtOAc in petroleum ether) to afford 4-(chloromethyl)-2-(4-chlorophenyl)thiazole (0.55 g, yield 77%) as white solid. ¹H NMR (400 MHz, CDCl₃) δ 7.91-7.88 (m, 2H), 7.44-7.41 (m, 2H), 7.33 (s, 1H), 4.75 (s, 2H). MS (ESI) m/z: Calculated for C₁₀H₇Cl₂NS: 242.97; found: 244.0 (M+H)⁺.

### 2-(2-(4-Chlorophenyl)thiazol-4-yl)acetonitrile

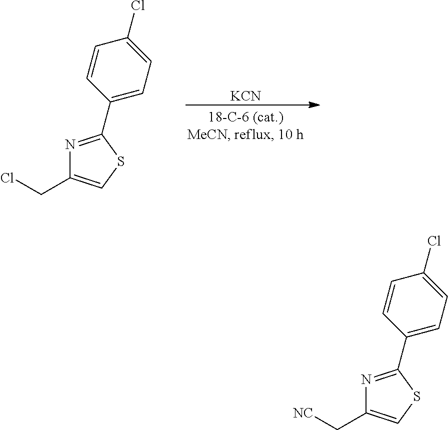

A catalytic amount of 18-crown-6-ether (20 mg) was added to a solution of 4-(chloromethyl)-2-(4-chlorophenyl)thiazole (0.55 g, 2.25 mmol) in acetonitrile (20 mL), followed by potassium cyanide (0.22 g, 3.37 mmol) and the reaction mixture was refluxed for 10 h. The reaction mixture was then quenched with water and the organic product extracted with EtOAc. The combined extracts were washed with H₂O and brine, dried over anhydrous sodium sulfate and concentrated under reduced pressure. The crude product was purified by column chromatography (silica gel 60-120 mesh, eluent 15% EtOAc in petroleum ether) to afford 2-(2-(4-chlorophenyl)thiazol-4-yl)acetonitrile (0.43 g, yield 82%) as off-white solid. ¹H NMR (300 MHz, CDCl₃) δ 7.89-7.86 (d, J=8.6 Hz, 2H), 7.45-7.42 (d, J=8.6 Hz, 2H), 7.32 (m, 1H), 3.96 (s, 2H). MS (ESI) m/z: Calculated for C₁₁H₇ClN₂S: 234.00; found: 235.0 (M+H)⁺.

### 4-(2-(4-Chlorophenyl)thiazol-4-yl)-1-methylpiperidine-4-carbonitrile

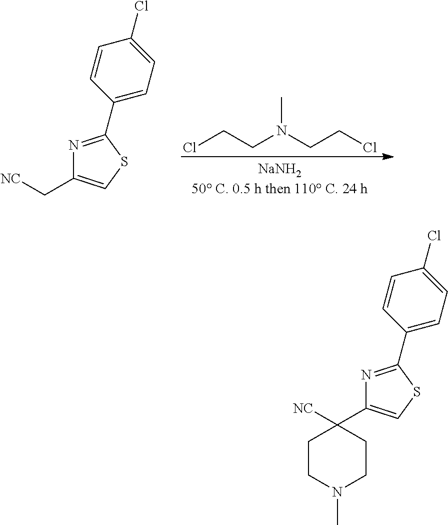

This compound was synthesized from 2-(2-(4-chlorophenyl)thiazol-4-yl)acetonitrile and bis-(2-chloroethyl)-methylamine hydrochloride as described in example 4 step 3 (350 mg, yield 32%). ¹H NMR (300 MHz, CDCl₃) δ 7.91-7.88 (d, J=8.6 Hz, 2H), 7.43-7.40 (d, J=8.6 Hz, 2H), 7.32 (s, 1H), 3.03-2.98 (m, 2H), 2.57-2.38 (m, 7H), 2.26-2.21 (m, 2H). MS (ESI) m/z: Calculated for C₁₆H₁₆ClN₃S: 317.08; found: 318.2 (M+H)⁺.

### (4-(2-(4-Chlorophenyl)thiazol-4-yl)-1-methylpiperidin-4-yl)methanamine

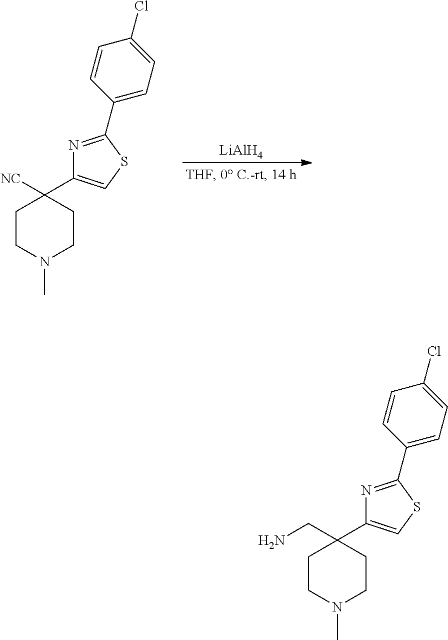

This compound was synthesized from 4-(2-(4-chlorophenyl)thiazol-4-yl)-1-methylpiperidine-4-carbonitrile as described in example 1 step 4 (220 mg, yield 47%). ¹H NMR (300 MHz, DMSO-d₆) δ 7.93-7.90 (d, J=8.6 Hz, 2H), 7.55-7.52 (d, J=8.6 Hz, 2H), 7.40 (s, 1H), 2.66 (s, 2H), 2.48-2.46 (m, 2H), 2.18-2.13 (m, 2H), 2.06 (s, 3H), 2.03-1.95 (m, 2H), 1.75-1.67 (m, 2H). MS (ESI) m/z: Calculated for C₁₆H₂₀ClN₃S: 321.11; found: 322.2 (M+H)⁺.

### N-((4-(2-(4-Chlorophenyl)thiazol-4-yl)-1-methylpiperidin-4-yl)methyl)-4-(5-(trifluoromethyl)-1,2,4-oxadiazol-3-yl)butanamide

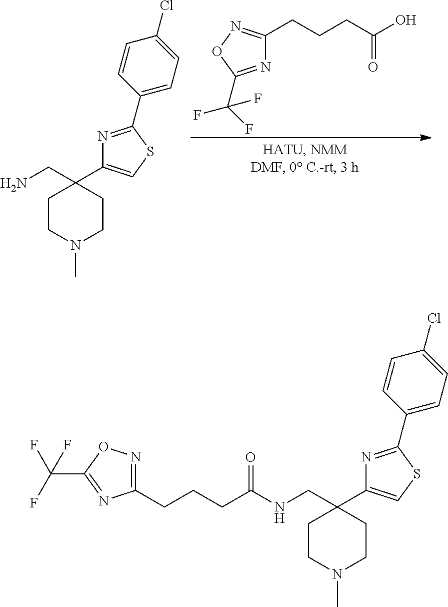

This compound was synthesized from (4-(2-(4-chlorophenyl)thiazol-4-yl)-1-methylpiperidin-4-yl)methanamine and 4-(5-(trifluoromethyl)-1,2,4-oxadiazol-3-yl)butanoic acid as described in example 1 step 5 (20 mg, yield 8%). ¹H NMR (400 MHz, MeOD) δ 7.97-7.95 (m, 2H), 7.48-7.46 (m, 3H), 3.50 (m, 2H), 2.85-2.79 (m, 4H), 2.69 (s, 3H), 2.57-2.47 (m, 3H), 2.27-2.23 (m, 2H), 2.08-1.96 (m, 5H). MS (ESI) m/z: Calculated for C₂₃H₂₅ClF₃N₆O₂S: 527.14; found: 528.1 (M+H)⁺.

### Example 9

### Methyl 2-(4-fluorophenyl)-4,5-dihydrooxazole-4-carboxylate

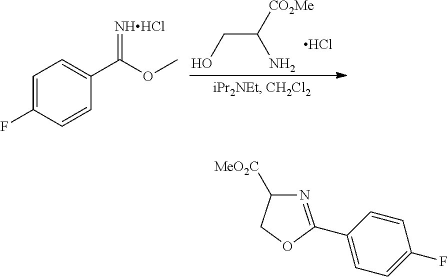

N,N-diisopropylethylamine (11 mL, 63.3 mmol) was added dropwise to a solution of methyl 4-fluorobenzimidate hydrochloride (10 g, 52.74 mmol) and DL-serine methyl ester HCl salt (9.9 g, 63.63 mmol) in dry CH₂Cl₂ (200 mL) at 0° C. The reaction mixture was stirred at room temperature for 24 h and then concentrated under reduced pressure. The reaction mixture was diluted with CH₂Cl₂ and the organic layer was washed with H₂O and brine, dried over anhydrous Na₂SO₄, and concentrated under reduced pressure to get methyl 2-(4-fluorophenyl)-4,5-dihydrooxazole-4-carboxylate (9.5 g, yield 81%) as an orange liquid. ¹H NMR (300 MHz, CDCl₃) δ 8.02-7.97 (m, 2H), 7.13-7.07 (t, J=8.7 Hz, 2H), 4.98-4.92 (m, 1H), 4.73-4.57 (m, 2H), 3.83 (s, 3H). MS (ESI) m/z: Calculated for C₁₁H₁₀,FNO₃: 223.06; found: 223.8 (M+H)⁺.

### Methyl 2-(4-fluorophenyl)oxazole-4-carboxylate

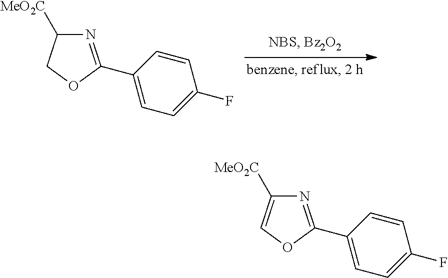

Benzoyl peroxide (0.49 g, 2.0 mmol) was added to a solution of methyl 2-(4-fluorophenyl)-4,5-dihydrooxazole-4-carboxylate (9.0 g, 40.3 mmol) in dry benzene (180 mL) and the mixture was refluxed for 15 min. N-bromosuccinimide (8.6 g, 48.3 mmol) was then added and the reaction mixture was refluxed for 2 h. The reaction mixture was quenched with ice-cold water and the crude product was extracted with EtOAc. The combined extracts were washed with 10% aqueous NaHCO₃ solution, H₂O and brine, dried over anhydrous Na₂SO₄, and concentrated under reduced pressure. The crude product was purified by column chromatography (silica 60-120 mesh, eluant 10-15% EtOAc in petroleum ether) to get methyl 2-(4-fluorophenyl)oxazole-4-carboxylate (6 g, yield 67%) as a white solid. ¹H NMR (400 MHz, CDCl₃) δ 8.29 (s, 1H), 8.14-8.10 (m, 2H), 7.19-7.15 (t, J=8.5 Hz, 2H), 3.96 (s, 3H). MS (ESI) m/z: Calculated for C₁₁H₈FNO₃: 221.05; found: 221.8 (M+H)⁺.

### (2-(4-Fluorophenyl)oxazol-4-yl)methanol

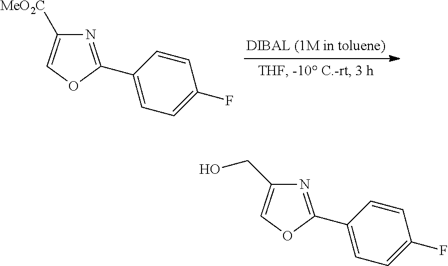

DIBAL-H (82 mL, 81.3 mmol, 1 M in toluene) was added dropwise to a solution of methyl 2-(4-fluorophenyl)oxazole-4-carboxylate (6.0 g, 27.13 mmol) in dry THF (200 mL) at −10° C. The reaction mixture was allowed to warm up to room temperature and stirred for 3 h. The reaction mixture was then quenched with saturated NH₄Cl solution, filtered through Celite, and extracted with EtOAc. The combined extracts were washed with 10% aqueous NaHCO₃ solution, H₂O and brine, dried over anhydrous Na₂SO₄, and concentrated under reduced pressure to get (2-(4-fluorophenyl)oxazol-4-yl)methanol (4.5 g, yield 86%) as a yellow solid. ¹H NMR (300 MHz, CDCl₃) δ 8.06-8.01 (m, 2H), 7.65 (s, 1H), 7.18-7.12 (t, J=8.7 Hz, 2H), 4.68 (s, 2H). MS (ESI) m/z: Calculated for C₁₀H₈FNO₂: 193.05; found: 193.8 (M+H)⁺.

### 2-(4-Fluorophenyl)oxazole-4-carbaldehyde

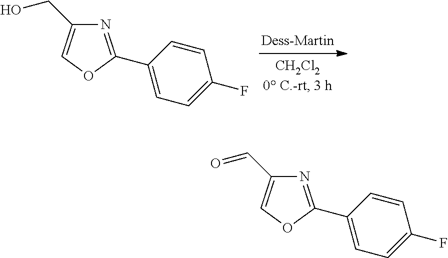

Dess-Martin periodinane (12.8 g, 30.2 mmol) was added to a solution of (2-(4-fluorophenyl)oxazol-4-yl)methanol (4.5 g, 23.29 mmol) in dry CH₂Cl₂ (90 mL) at 0° C. The reaction mixture was stirred at room temperature for 3 h. The reaction mixture was then quenched with saturated NaHCO₃ solution at 0° C. The organic product was extracted with EtOAc. The combined extracts were washed with saturated sodium thiosulfate solution and brine. Solvent was removed under reduced pressure and the crude product was purified by column chromatography (silica gel 60-120 mesh, eluent 10% EtOAc in petroleum ether) to afford 2-(4-fluorophenyl)oxazole-4-carbaldehyde (2.8 g, yield 63%) as a white solid. ¹H NMR (300 MHz, CDCl₃) δ 10.01 (s, 1H), 8.32 (s, 1H), 8.14-8.10 (m, 2H), 7.23-7.17 (t, J=8.8 Hz, 2H). MS (ESI) m/z: Calculated for C₁₀H₆FNO₂: 191.04; found: 191.8 (M+H)⁺.

### 2-(2-(4-Fluorophenyl)oxazol-4-yl)-2-hydroxyacetonitrile

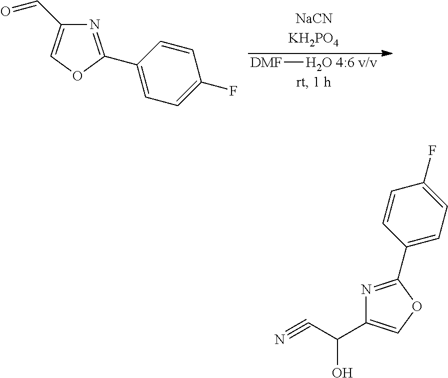

KH₂PO₄ (712 mg, 5.23 mmol) and NaCN (251 mg, 5.12 mmol) were added to a solution of 2-(4-fluorophenyl)oxazole-4-carbaldehyde (500 mg, 2.62 mmol) in DMF-H₂O (10 mL, 4:6, v/v). The resulting reaction mixture was stirred at room temperature for 1 h, then diluted with water and extracted with EtOAc. The combined extracts were washed with H₂O and brine, dried over anhydrous Na₂SO₄, and concentrated under reduced pressure to afford 2-(2-(4-fluorophenyl)oxazol-4-yl)-2-hydroxyacetonitrile (500 mg, yield 87%). ¹H NMR (400 MHz, CDCl₃) δ 8.07-8.02 (m, 2H), 7.88 (s, 1H), 7.20-7.16 (t, J=8.8 Hz, 2H), 5.62 (s, 1H), 4.38 (br s, 1H). MS (ESI) m/z: Calculated for C₁₁H₇FN₂O₂: 218.05; found: 218.8 (M+H)⁺.

### 2-Amino-1-(2-(4-fluorophenyl)oxazol-4-yl)ethanol

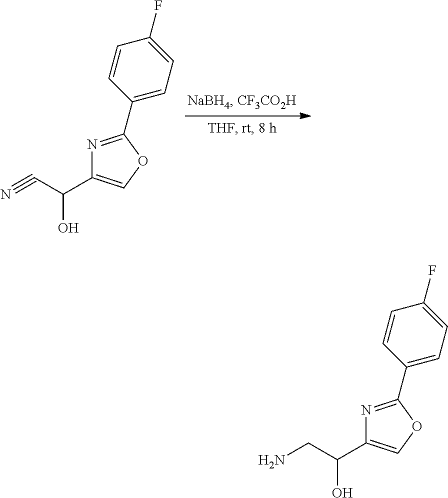

Trifluoroacetic acid (0.35 mL, 4.60 mmol) was added dropwise to a suspension of NaBH₄ (0 174 g, 4.60 mmol) in dry THF (10 mL) at 0° C., followed by addition of 2-(2-(4-fluorophenyl)oxazol-4-yl)-2-hydroxyacetonitrile (0 20 g, 0.92 mmol) also portionwise. The reaction mixture was stirred at room temperature for 8 h, and then concentrated under reduced pressure and diluted with ice-water. The mixture was acidified to pH ˜2 using 1.5N HCl at 0° C. and then heated to 50° C. for 20 min. The solution was basified with aqueous NH₄OH solution and the organic product was extracted with CHCl₃. The combined extracts were washed with brine and concentrated under reduced pressure to afford 2-amino-1-(2-(4-fluorophenyl)oxazol-4-yl) ethanol (120 mg, crude), which was carried through without further purification. MS (ESI) m/z: Calculated for C₁₁H₁₁FN₂O₂: 222.08; found: 222.8 (M+H)⁺.

### N-(2-(2-(4-Fluorophenyl)oxazol-4-yl)-2-hydroxyethyl)-4-(5-(trifluoromethyl)-1,2,4-oxadiazol-3-yl)butanamide

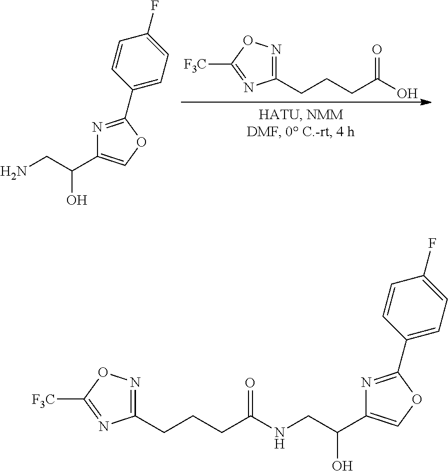

This compound was synthesized from 2-amino-1-(2-(4-fluorophenyl)oxazol-4-yl)ethanol and 4-(5-(trifluoromethyl)-1,2,4-oxadiazol-3-yl)butanoic acid as described in example 1 step 5 (65 mg, yield 34%). ¹H NMR (400 MHz, MeOD) δ 8.08-8.05 (m, 2H), 7.88-7.87 (d, J=1.0 Hz, 1H), 7.27-7.22 (t, J=8.9 Hz, 2H), 4.82-4.79 (t, J=6.0 Hz, 1H), 3.68-3.63 (m, 1H), 3.57-3.52 (m, 1H), 2.89-2.85 (t, J=7.4 Hz, 2H), 2.35-2.31 (m, 2H), 2.11-2.04 (m, 2H). MS (ESI) m/z: Calculated for C₁₈H₁₆F₄N₄O₄: 428.11; found: 429.1 (M+H)⁺.

### Example 10

### 5-Amino-5-(hydroxyimino)pentanoic acid

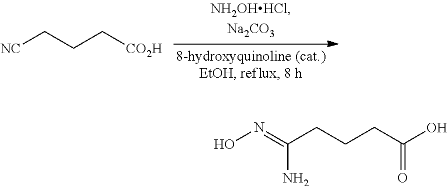

8-Hydroxyquinoline (380 mg) was added to a solution of 4-cyanobutyric acid (6.0 g, 53.1 mmol) in ethanol (180 mL). Hydroxylamine hydrochloride (7.8 g, 112.1 mmol) in water (30 mL), followed by sodium carbonate (9.0 g, 84.9 mmol) in water (30 mL) were then added to the reaction mixture. The mixture was heated to reflux for 8 h monitored by TLC (CHCl₃:MeOH 9:1 v/v). After removal of ethanol under reduced pressure, the reaction mixture was acidified with 1.5 N HCl, and concentrated under reduced pressure to afford 5-amino-5-(hydroxyimino)pentanoic acid (12.0 g, crude), which was carried through without further purification. ¹H NMR (300 MHz, DMSO-d₆) δ 12.63 (br s, 1H), 10.95 (br s, 1H), 10.43 (br s, 2H), 2.42-2.38 (m, 2H), 2.26-2.23 (m, 2H), 1.86-1.79 (m, 2H).

### 4-(5-(Difluoromethyl)-1,2,4-oxadiazol-3-yl)butanoic acid

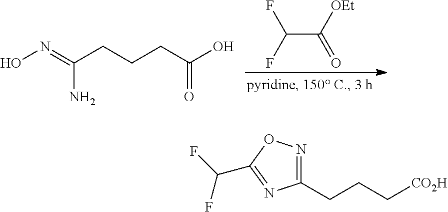

A solution of 5-amino-5-(hydroxyimino)pentanoic acid (1.0 g, 6.84 mmol) in anhydrous pyridine (10 mL) was cooled to 0° C. and difluoroethyl acetate (4.0 mL) was added dropwise. The reaction mixture was slowly warmed to room temperature and further heated to 105° C. for 3 h. The reaction mixture was concentrated under reduced pressure and acidified with 1.5 N HCl. The organic product was extracted with EtOAc and the combined extracts were was washed with 1.5 N HCl, water and brine. Solvent was removed under reduced pressure and the crude product was purified by column chromatography (silica gel 60-120 mesh, eluant: 25-30% EtOAc in petroleum ether) to afford -(5-(difluoromethyl)-1,2,4-oxadiazol-3-yl)butanoic acid (200 mg, yield 22%) as colorless viscous liquid. ¹H NMR (300 MHz, DMSO-d₆) δ 12.20 (br s, 1H), 7.61-7.27 (m, 1H), 2.85-2.80 (t, J=7.5 Hz, 2H), 2.33-2.29 (t, J=7.3 Hz, 2H), 1.92-1.85 (m, 2H).

### 4-(5-(Difluoromethyl)-1,2,4-oxadiazol-3-yl)-N-(4-(dimethylamino)-2-(2-(4-fluorophenyl)oxazol-4-yl)butyl)butanamide

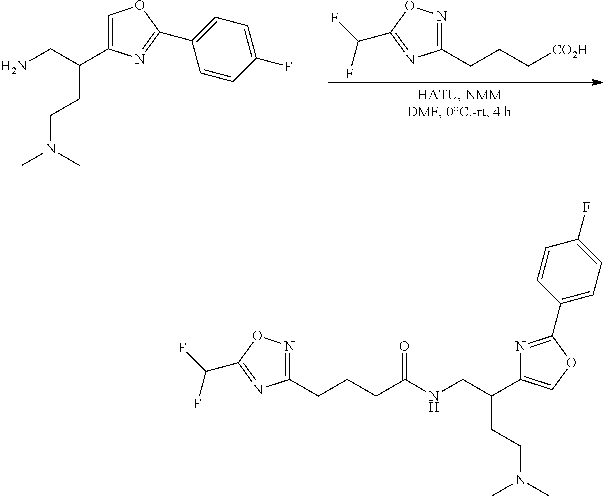

This compound was synthesized from 3-(2-(4-fluorophenyl)oxazol-4-yl)-N1,N1-dimethylbutane-1,4-diamine and 4-(5-(difluoromethyl)-1,2,4-oxadiazol-3-yl)butanoic acid as described in example 1 step 5 (17 mg, yield 6%). ¹H NMR (400 MHz, MeOD) δ 8.08-8.04 (m, 2H), 7.85 (s, 1H), 7.27-6.99 (m, 3H), 3.57-3.53 (m, 1H), 3.48-3.43 (m, 1H), 3.15-3.07 (m, 1H), 3.03-2.94 (m, 2H), 2.84-2.77 (m, 8H), 2.31-2.27 (m, 2H), 2.12-2.02 (m, 4H). MS (ESI) m/z: Calculated for C₂₂H₂₆F₃N₅O₃: 465.20; found: 466.2 (M+H)⁺.

### Example 11

### 3-Cyano-N-(2-(2-(4-fluorophenyl)oxazol-4-yl)-2-methylpropyl)propane-1-sulfonamide

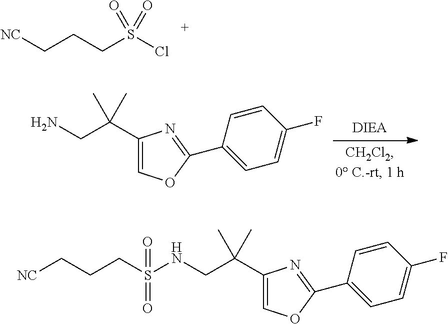

DIEA (0.8 mL, 4.5 mmol) was added to a solution of 2-(2-(4-fluorophenyl)oxazol-4-yl)-2-methylpropan-1-amine (384 mg, 1.64 mmol) in CH₂Cl₂ (2.5 mL), followed by 3-cyanopropane-1-sulfonylchloride (250 mg, 1.4 mmol) at 0° C. The mixture was allowed to warm up to room temperature and further stirred for 1 h. The reaction mixture was diluted with CH₂Cl₂ and the organic layer was washed with water and brine, dried over anhydrous sodium sulfate, and concentrated under reduced pressure. The crude product was purified by column chromatography (silica gel 60-120 mesh, eluent 30-40% EtOAc in petroleum ether) to afford 3-cyano-N-(2-(2-(4-fluorophenyl)oxazol-4-yl)-2-methylpropyl)propane-1-sulfonamide (170 mg, yield 31%). ¹H NMR (300 MHz, CDCl₃) δ 8.03-7.99 (m, 2H), 7.46 (s, 1H), 7.19-7.13 (t, J=8.7 Hz, 2H), 3.28-3.26 (d, J=5.9 Hz, 2H), 3.16-3.11 (t, J=7.2 Hz, 2H), 2.59-2.55 (t, J=7.0 Hz, 2H), 2.18-2.13 (m, 2H), 1.37 (s, 6H). MS (ESI) m/z: Calculated for C₁₇H₂₀FN₃O₃S: 365.12; found: 364.2 (M−H)⁻.

### 4-(N-(2-(2-(4-Fluorophenyl)oxazol-4-yl)-2-methylpropyl)sulfamoyl)-N′-hydroxybutanimidamide

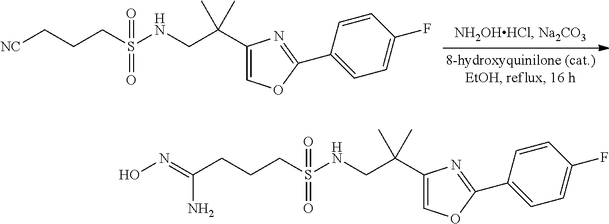

This compound was synthesized from 3-cyano-N-(2-(2-(4-fluorophenyl)oxazol-4-yl)-2-methylpropyl)propane-1-sulfonamide as described in example 1 step 6 (85 mg, crude) and it was carried through without further purification. ¹H NMR (400 MHz, DMSO-d₆) δ 8.79 (s, 1H), 8.03-8.00 (m, 2H), 7.95 (s, 1H), 7.39-7.34 (t, J=9.0 Hz, 2H), 6.98-6.95 (t, J=6.7 Hz, 1H), 5.37 (br s, 2H), 3.09-3.07 (d, J=6.7 Hz, 2H), 2.94-2.90 (m, 2H), 2.05-2.01 (m, 2H), 1.86-1.79 (m, 2H), 1.23 (s, 6H). MS (ESI) m/z: Calculated for C₁₇H₂₃FN₄O₄S: 398.14; found: 397.6 (M−H)⁻.

### N-(2-(2-(4-Fluorophenyl)oxazol-4-yl)-2-methylpropyl)-3-(5-(trifluoromethyl)-1,2,4-oxadiazol-3-yl)propane-1-sulfonamide

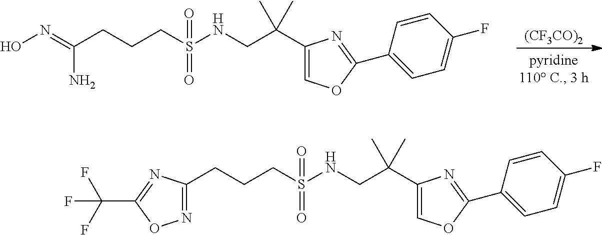

This compound was synthesized from 4-(N-(2-(2-(4-fluorophenyl)oxazol-4-yl)-2-methylpropyl)sulfamoyl)-N′-hydroxybutanimidamide as described in example 1 step 7 (170 mg, yield 37%). ¹H NMR (400 MHz, MeOD) δ 8.07-8.03 (m, 2H), 7.74 (s, 1H), 7.25-7.21 (t, J=8.8 Hz, 2H), 3.27 (s, 2H), 3.13-3.09 (m, 2H), 3.00-2.96 (t, J=7.5 Hz, 2H), 2.21-2.14 (m, 2H), 1.33 (s, 6H). MS (ESI) m/z: Calculated for C₁₉H₂₀F₄N₄O₄S: 476.11; found: 475.4 (M−H)⁻.

### Example 12

### 1-(2-(4-Fluorophenyl)oxazol-4-yl)ethanone

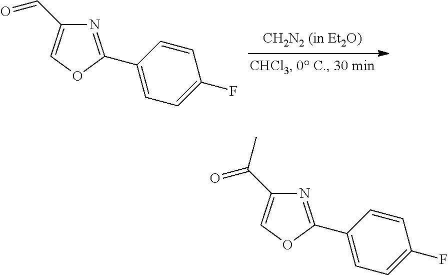

A freshly prepared solution of diazomethane in diethyl ether (80 mL) was added to a solution of 2-(4-fluorophenyl)oxazole-4-carbaldehyde (2.0 g, 10.4 mmol) in dry CHCl₃ (40 mL) at 0° C. The reaction mixture was stirred at for 30 min at 0° C. and then it was quenched with saturated aqueous NaHCO₃ solution. The reaction mixture was diluted with CHCl₃ and the organic layer was washed with water and brine, dried over anhydrous sodium sulfate, and concentrated under reduced pressure. The crude product was purified by column chromatography (silica gel 60-120 mesh, eluent 8-12% EtOAc in petroleum ether) to afford 1-(2-(4-fluorophenyl)oxazol-4-yl)ethanone (1.4 g, yield 67%) as white solid. ¹H NMR (300 MHz, CDCl₃) δ 8.25 (s, 1H), 8.13-8.08 (m, 2H), 7.21-7.15 (t, J=8.8 Hz, 2H), 2.60 (s, 3H). MS (ESI) m/z: Calculated for C₁₁H₈FNO₂: 205.05; found: 205.9 (M+H)⁺.

### 3-(2-(4-Fluorophenyl)oxazol-4-yl)-3-hydroxypropanenitrile

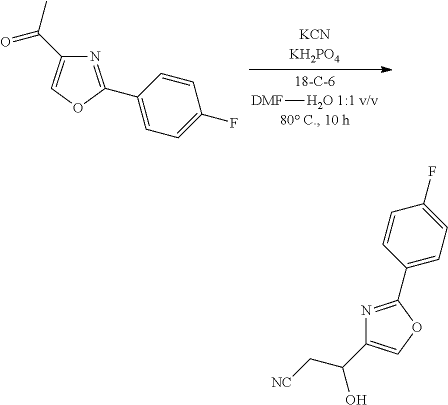

KH₂PO₄ (1.9 g, 13.7 mmol), KCN (655 mg, 10.2 mmol) and catalytic 18-crown-6 (100 mg, 0.1 mmol) were added to a solution of 1-(2-(4-fluorophenyl)oxazol-4-yl)ethanone (1.4 g, 6.82 mmol) in DMF-H₂O (28 mL, 1:1, v/v). The resulting reaction mixture was stirred at 80° C. for 10 h, diluted with water and extracted with EtOAc. The combined extracts were washed with H₂O and brine, dried over anhydrous Na₂SO₄, and concentrated under reduced pressure. The crude product was purified by column chromatography (silica gel 60-120 mesh, eluent 40-50% EtOAc in petroleum ether) to afford 3-(2-(4-fluorophenyl)oxazol-4-yl)-3-hydroxypropanenitrile (300 mg, yield 19%) as pale yellow solid. ¹H NMR (300 MHz, CDCl₃) δ 8.05-8.00 (m, 2H), 7.27 (s, 1H), 7.21-7.18 (m, 2H), 5.12 (m, 1H), 2.99-2.96 (m, 2H). MS (ESI) m/z: Calculated for C₁₂H₉FN₂O₂: 232.06; found: 232.9 (M+H)⁺.

### 3-Amino-1-(2-(4-fluorophenyl)oxazol-4-yl)propan-1-ol

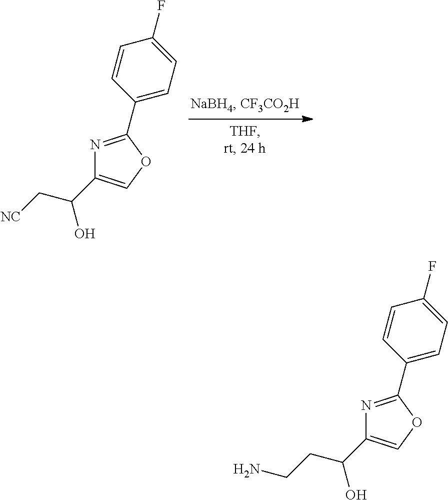

This compound was synthesized from 3-(2-(4-fluorophenyl)oxazol-4-yl)-3-hydroxypropanenitrile as described in example 9 step 6 (200 mg, yield 50%) and it was carried through without further purification. ¹H NMR (300 MHz, DMSO-d₅) δ 8.01-7.94 (m, 3H), 7.38-7.32 (t, J=8.9 Hz, 2H), 4.70-4.65 (m, 1H), 2.75-2.66 (m, 2H), 1.88-1.68 (m, 2H). MS (ESI) m/z: Calculated for C₁₂H₁₃FN₂O₂: 236.10; found: 236.9 (M+H)⁺.

### 3-Cyano-N-(3-(2-(4-fluorophenyl)oxazol-4-yl)-3-hydroxypropyl)propanamide

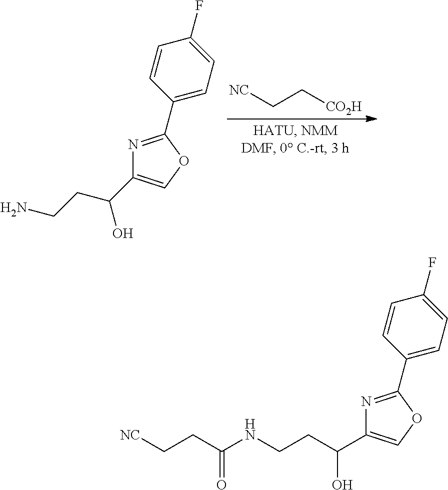

This compound was synthesized from 3-amino-1-(2-(4-fluorophenyl)oxazol-4-yl)propan-1-ol and 3-cyanopropanoic acid as described in example 1 step 5 (150 mg, yield 56%) as pale yellow solid. ¹H NMR (300 MHz, MeOD) δ 8.07-8.02 (m, 2H), 7.82 (s, 1H), 7.26-7.20 (t, J=8.9 Hz, 2H), 4.75-4.71 (m, 1H), 3.42-3.33 (m, 2H), 2.71-2.66 (m, 2H), 2.55-2.50 (m, 2H), 2.13-1.91 (m, 2H). MS (ESI) m/z: Calculated for C₁₆H₁₆FN₃O₃: 317.12; found: 318.1 (M+H)⁺.

### 4-Amino-N-(3-(2-(4-fluorophenyl)oxazol-4-yl)-3-hydroxypropyl)-4-(hydroxyimino)butanamide

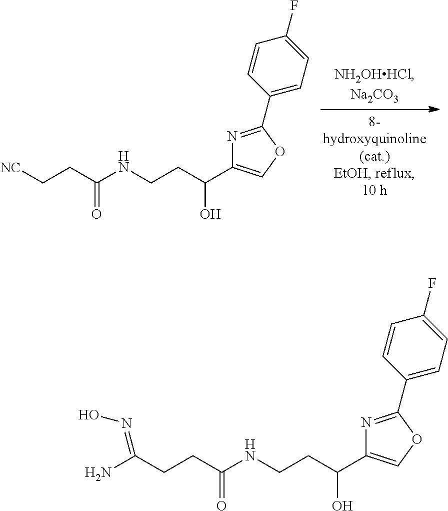

This compound was synthesized from 3-cyano-N-(3-(2-(4-fluorophenyl)oxazol-4-yl)-3-hydroxypropyl)propanamide as described in example 1 step 6 (90 mg, crude), and it was carried through without further purification. MS (ESI) m/z: Calculated for C₁₆H₁₉FN₄O₄: 350.14; found: 351.1 (M+H)⁺.

### N-(3-(2-(4-Fluorophenyl)oxazol-4-yl)-3-hydroxypropyl)-3-(5-(trifluoromethyl)-1,2,4-oxadiazol-3-yl)propanamide

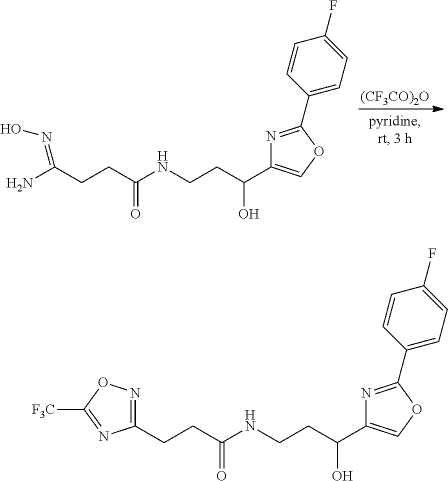

This compound was synthesized from 4-amino-N-(3-(2-(4-fluorophenyl)oxazol-4-yl)-3-hydroxypropyl)-4-(hydroxyimino)butanamide as described in example 1 step 7 (20 mg, yield 18%). ¹H NMR (400 MHz, MeOD) δ 8.08-8.05 (m, 2H), 7.83 (d, J=0.8 Hz, 1H), 7.27-7.23 (t, J=8.8 Hz, 2H), 4.74-4.71 (m, 1H), 3.41-3.35 (m, 2H), 3.18-3.14 (t, J=7.3 Hz, 2H), 2.73-2.70 (t, J=7.3 Hz, 2H), 2.13-2.05 (m, 1H), 2.01-1.92 (m, 1H). MS (ESI) m/z: Calculated for C₁₈H₁₆F₄N₄O₄: 428.11; found: 429.1 (M+H)⁺.

### Example 13

### 4-Oxo-4-(thiophen-2-yl)butanoic acid

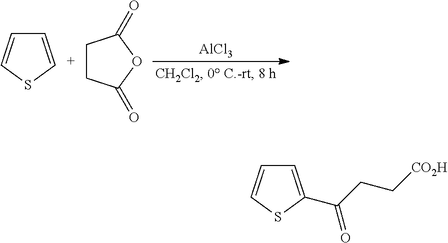

Anhydrous aluminium chloride (15.8 g, 0.12 mol) was added to a solution of succinic anhydride (11.9 g, 0.12 mol) in dry CH₂Cl₂ (50 Ml) and the reaction mixture was cooled to 0° C. A solution of thiophene (10.0 g, 0.12 mol) in CH₂Cl₂ (50 Ml) was then added dropwise maintaining the same temperature. The reaction mixture was allowed to warm up to room temperature and stirred for 8 h. The mixture was then cooled to 0° C. and the Ph was adjusted to ˜3 using 6N HCl. The organic product was extracted with CH₂Cl₂. The combined extracts were washed with water and brine, dried over anhydrous sodium sulfate, and concentrated under reduced pressure. The crude product was purified by column chromatography (silica 60-120 mesh, eluant 5-10% MeOH in CH₂Cl₂), to get 4-oxo-4-(thiophen-2-yl)butanoic acid (8.3 g, yield 38%). ¹H NMR (400 MHz, DMSO-d₆) δ 12.18 (s, 1H), 8.00-7.98 (m, 2H), 7.26-7.23 (m, 1H), 3.21-3.18 (m, 2H), 2.58-2.55 (m, 2H). MS (ESI) m/z: Calculated for C₈H₈O₃S: 184.02; found: 184.9 (M+H)⁺.

### 4-(Thiophen-2-yl)butanoic acid

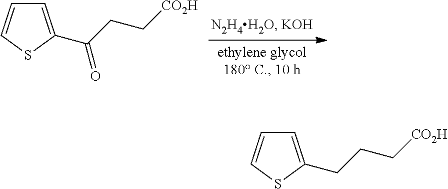

Hydrazine hydrate (99%) (2.2 Ml, 45.9 mmol) and KOH pellets (2.37 g, 42.4 mmol) were added to a solution of 4-oxo-4-(thiophen-2-yl)butanoic acid (2.3 g, 12.48 mmol) in ethylene glycol (30 Ml), and the reaction mixture was heated to 180° C. for 10 h. The reaction mixture was cooled to room temperature and diluted with water. The aqueous layer was washed with diethyl ether, acidified with 6N HCl and then extracted with diethyl ether. The organic layer was then dried over anhydrous sodium sulfate and concentrated under reduced pressure. The crude product was purified by column chromatography (silica 60-120 mesh, eluant 2% MeOH in CH₂Cl₂), to get 4-(thiophen-2-yl)butanoic acid (1.8 g, yield 85%). ¹H NMR (300 MHz, DMSO-d₆) δ 12.06 (s, 1H), 7.31-7.29 (m, 1H), 6.94-6.91 (m, 1H), 6.84-6.82 (m, 1H), 2.82-2.77 (t, J=7.7 Hz, 2H), 2.27-2.22 (t, J=7.3 Hz, 2H), 1.86-1.76 (m, 2H). MS (ESI) m/z: Calculated for C₈H₁₀O₂S: 170.04; found: 170.8 (M+H)⁺.

### Methyl 4-(thiophen-2-yl)butanoate

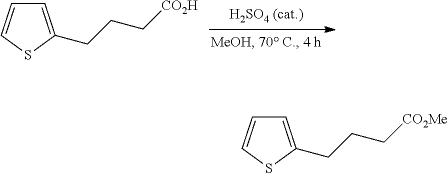

A catalytic amount of conc. H₂SO₄ (1 Ml) was added to a solution of 4-(thiophen-2-yl)butanoic acid (1.2 g, 7.05 mmol) in dry MeOH (30 Ml) at 0° C. The resulting reaction mixture was heated to 70° C. for 4 h, cooled to room temperature, and concentrated under reduced pressure. The mixture was then diluted with water and extracted with EtOAc. The combined extracts were washed with water and brine, dried over anhydrous sodium sulfate, and concentrated under reduced pressure. The crude product was purified by column chromatography (silica 60-120 mesh, eluant 5-10% EtOAc in petroleum ether) to get methyl 4-(thiophen-2-yl)butanoate (1.1 g, yield 89%). ¹H NMR (300 MHz, CDCl₃) δ 7.14-7.12 (m, 1H), 6.94-6.91 (m, 1H), 6.81-6.80 (m, 1H), 3.68 (s, 3H), 2.91-2.86 (t, J=7.5 Hz, 2H), 2.41-2.36 (m, 2H), 2.07-1.97 (m, 2H).

### Methyl 4-(5-formylthiophen-2-yl)butanoate

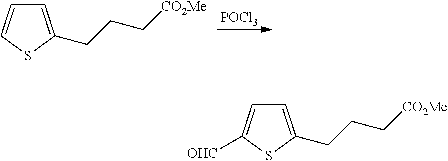

Freshly distilled POCl₃ (0.4 Ml, 4.12 mmol) was added to a solution of methyl 4-(thiophen-2-yl)butanoate (1.1 g, 5.97 mmol) in dry DMF (0.7 Ml) at 0° C. The reaction mixture was further heated to 110° C. for 1.5 h, then cooled to room temperature and quenched with ice water. The Ph of the reaction mixture was adjusted to ˜7 using aqueous Na₂CO₃ solution. The organic product was extracted with diethyl ether and the combined extracts were washed with water and brine, dried over anhydrous sodium sulfate and concentrated under reduced pressure to get methyl 4-(5-formylthiophen-2-yl)butanoate (1.0 g, yield 83%), which was carried through without further purification. ¹H NMR (300 MHz, CDCl₃) δ 9.82 (s, 1H), 7.63-7.61 (d, J=3.7 Hz, 1H), 6.94-6.92 (m, 1H), 3.68 (s, 3H), 2.96-2.91 (t, J=7.5 Hz, 2H), 2.42-2.37 (m, 2H), 2.09-1.99 (m, 2H). MS (ESI) m/z: Calculated for C₁₀H₁₂O₃S: 212.05; found: 212.9 (M+H)⁺.

### 4-(5-(2,2,2-Trifluoro-1-hydroxyethyl)thiophen-2-yl)butanoate

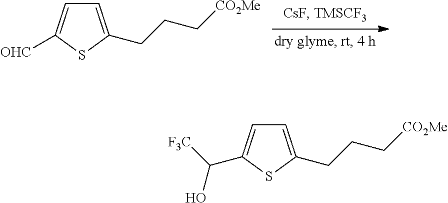

CsF (70 mg, 0.47 mmol) was added to a solution of methyl 4-(5-formylthiophen-2-yl)butanoate (1.0 g, 4.71 mmol) in dry 1,2-dimethoxyethane (5 Ml) at 0° C., followed by trifluoromethyl trimethylsilane (0.8 Ml, 5.65 mmol) dropwise. The reaction mixture was stirred at room temperature for 4 h, quenched with 3N HCl, and stirred for further 30 minutes. The crude product was extracted with EtOAc. The combined extracts were washed with brine, dried over anhydrous Na₂SO₄, and concentrated under reduced pressure. The crude product was purified by column chromatography (silica 60-120 mesh, eluant 10% EtOAc in petroleum ether) to get methyl 4-(5-(2,2,2-trifluoro-1-hydroxyethyl)thiophen-2-yl)butanoate (0.63 g, yield 47%) as yellow liquid. ¹H NMR (400 MHz, CDCl₃) δ 7.02-7.01 (d, J=3.5 Hz, 1H), 6.74-6.73 (m, 1H), 5.23-5.17 (m, 1H), 3.68 (s, 3H), 2.88-2.84 (t, J=7.5 Hz, 2H), 2.81-2.80 (d, J=5.3 Hz, 1H), 2.40-2.36 (t, J=7.5 Hz, 2H), 2.05-1.97 (m, 2H).

### Methyl 4-(5-(2,2,2-trifluoroacetyl)thiophen-2-yl)butanoate

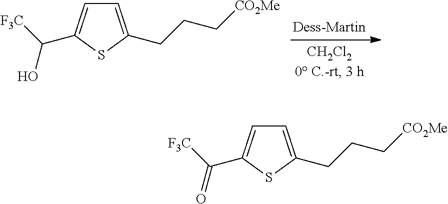

This compound was synthesized from methyl 4-(5-(2,2,2-trifluoro-1-hydroxyethyl)thiophen-2-yl)butanoate as described in example 9 step 4 (550 mg, yield 79%). ¹H NMR (300 MHz, CDCl₃) δ 7.83-7.81 (m, 1H), 6.98-6.97 (m, 1H), 3.70 (s, 3H), 3.00-2.95 (t, J=7.6 Hz, 2H), 2.43-2.39 (m, 2H), 2.12-2.02 (m, 2H). MS (ESI) m/z: Calculated for C₁₁H₁₁F₃O₃S: 280.04; found: 279.6 (M−H)⁻.

### 4-(5-(2,2,2-Trifluoroacetyl)thiophen-2-yl)butanoic acid

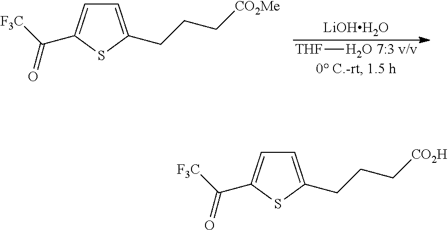

Methyl 4-(5-(2,2,2-trifluoroacetyl)thiophen-2-yl)butanoate (250 mg, 0.89 mmol) was dissolved in THF—H₂O (15 Ml, 2:1 v/v) and cooled to 0° C. LiOH.H₂O (35 mg, 0.89 mmol) was added and the reaction mixture was allowed to warm up to room temperature and stirred for 1.5 h. Solvent was removed under reduced pressure and the aqueous layer was washed with EtOAc. The Ph of the aqueous layer was adjusted to 2-3 using 1.5N HCl. The product was extracted with EtOAc. The combined extracts were dried over anhydrous sodium sulfate and concentrated under reduced pressure to get 4-(5-(2,2,2-trifluoroacetyl)thiophen-2-yl)butanoic acid (220 mg, crude), which was carried through without further purification. ¹H NMR (300 MHz, DMSO-d₆) δ 12.13 (br s, 1H), 8.00-7.98 (m, 1H), 7.21-7.19 (d, J=3.9 Hz, 1H), 2.98-2.93 (t, J=7.7 Hz, 2H), 2.31-2.26 (m, 2H), 1.93-1.83 (m, 2H). MS (ESI) m/z: Calculated for C₁₀H₉F₃O₃S: 266.02; found: 264.8 (M−H)⁻.

### N-(2-(2-(4-Fluorophenyl)oxazol-4-yl)-2-methylpropyl)-4-(5-(2,2,2-trifluoroacetyl)thiophen-2-yl)butanamide

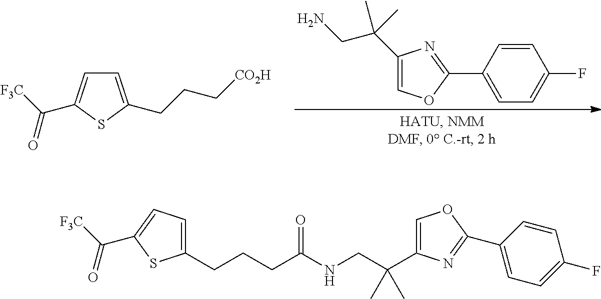

This compound was synthesized from 2-(2-(4-fluorophenyl)oxazol-4-yl)-2-methylpropan-1-amine and 4-(5-(2,2,2-trifluoroacetyl)thiophen-2-yl)butanoic acid as described in example 1 step 5 (40 mg, yield 15%). ¹H NMR (400 MHz, DMSO-d₆, 80° C.) δ 8.01-7.97 (m, 2H), 7.95-7.94 (m, 1H), 7.82 (s, 1H), 7.37 (br s, 1H), 7.32-7.28 (t, J=9.0 Hz, 2H), 7.15-7.14 (d, J=4.0 Hz, 1H), 3.33-3.32 (d, J=6.4 Hz, 2H), 2.94-2.91 (t, J=7.6 Hz, 2H), 2.23-2.19 (m, 2H), 1.95-1.88 (m, 2H), 1.24 (s, 6H). MS (ESI) m/z: Calculated for C₂₃H₂₂F₄N₂O₃S: 482.13; found: 483.1 (M+H)⁺.

### Example 14

### 2-(2-(4-Fluorophenyl)oxazol-4-yl)ethanamine

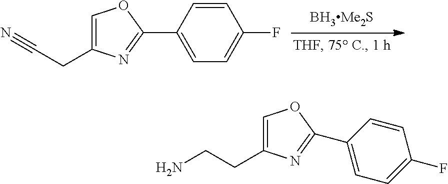

This compound was synthesized from 2-(2-(4-fluorophenyl)oxazol-4-yl)acetonitrile as described in example 7 step 1 (150 mg, yield 74%). ¹H NMR (400 MHz, DMSO-d₆) δ 8.03-7.99 (m, 3H), 7.40-7.35 (t, J=8.9 Hz, 2H), 2.93-2.90 (m, 2H), 2.69-2.65 (m, 2H). MS (ESI) m/z: Calculated for C₁₁H₁₁FN₂O: 206.09; found: 207.0 (M+H)⁺.

### N-(2-(2-(4-Fluorophenyl)oxazol-4-yl)ethyl)-4-(5-(2,2,2-trifluoroacetyl)thiophen-2-yl)butanamide

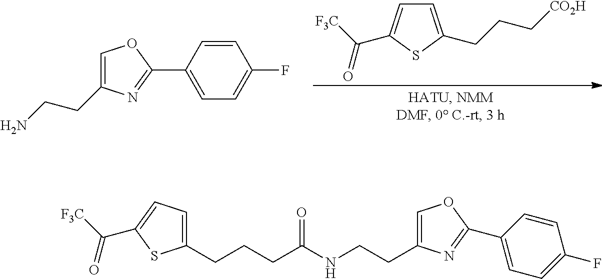

This compound was synthesized from 2-(2-(4-fluorophenyl)oxazol-4-yl) ethanamine and 4-(5-(2,2,2-trifluoroacetyl)thiophen-2-yl)butanoic acid as described in example 1 step 5 (54 mg, yield 17%). ¹H NMR (400 MHz, DMSO-d₆) δ 8.00-7.94 (m, 3H), 7.86 (s, 1H), 7.63 (m, 1H), 7.33-7.28 (t, J=9.0 Hz, 2H), 7.16-7.14 (m, 1H), 3.41-3.36 (m, 2H), 2.96-2.92 (t, J=7.5 Hz, 2H), 2.72-2.68 (t, J=7.5 Hz, 2H), 2.20-2.16 (m, 2H), 1.97-1.89 (m, 2H). MS (ESI) m/z: Calculated for C₂₁H₁₈F₄N₂O₃S: 454.10; found: 455.1 (M+H)⁺.

### Example 15

### 2-(3-(4-Fluorophenyl)-1H-1,2,4-triazol-5-yl)-2-methylpropanenitrile

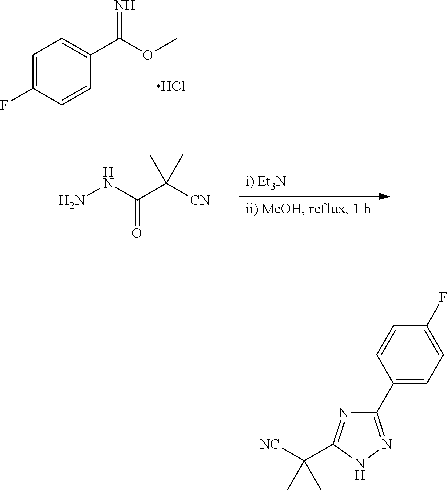

2-Cyano-2-methylpropanehydrazide (500 mg, 3.9 mmol) and Et₃N (0.5 mL, 3.9 mmol) were added to a solution of methyl 4-fluorobenzimidate (750 mg, 3.9 mmol) in dry MeOH (10 mL) and the mixture was heated to reflux for 1 h. The reaction mixture was concentrated under reduced pressure and the crude product was purified by column chromatography (silica 60-120 mesh, eluant 10-15% EtOAc in petroleum ether) to get 2-(3-(4-fluorophenyl)-1H-1,2,4-triazol-5-yl)-2-methylpropanenitrile (550 mg, yield 60%). ¹H NMR (300 MHz, MeOD) δ 8.03-7.98 (m, 2H), 7.29-7.23 (m, 2H), 1.81 (s, 6H). MS (ESI) m/z: Calculated for C₁₂H₁₁FN₄: 230.10; found: 231.2 (M+H)⁺.

### 2-(3-(4-Fluorophenyl)-1H-1,2,4-triazol-5-yl)-2-methylpropan-1-amine

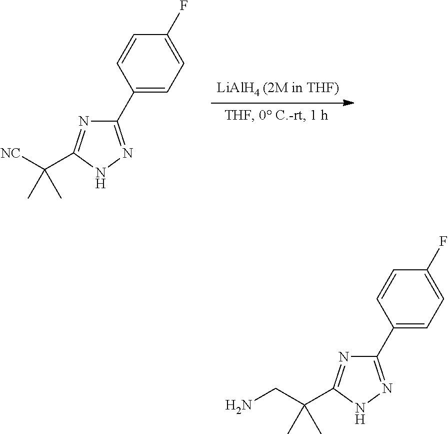

This compound was synthesized from 2-(3-(4-fluorophenyl)-1H-1,2,4-triazol-5-yl)-2-methylpropanenitrile as described in example 1 step 4 (100 mg, crude) and it was carried through without further purification. MS (ESI) m/z: Calculated for C₁₂H₁₅FN₄: 234.13; found: 233.1 (M−H)⁻.

### N-(2-(3-(4-Fluorophenyl)-1H-1,2,4-triazol-5-yl)-2-methylpropyl)-4-(5-(2,2,2-trifluoroacetyl)thiophen-2-yl)butanamide

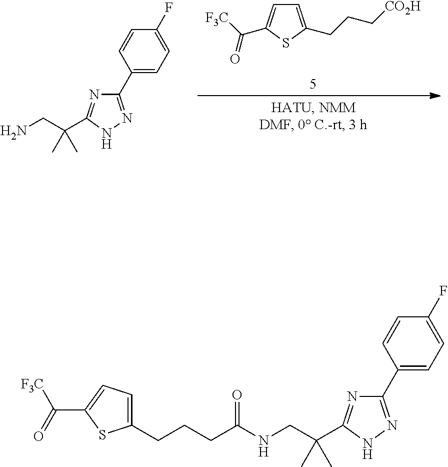

This compound was synthesized from 2-(3-(4-fluorophenyl)-1H-1,2,4-triazol-5-yl)-2-methylpropan-1-amine and 4-(5-(2,2,2-trifluoroacetyl)thiophen-2-yl)butanoic acid as described in example 1 step 5 (70 mg, yield 38%). ¹H NMR (400 MHz, DMSO-d₆) δ 13.75 (br s, 1H), 8.02-7.98 (dd, J=8.4 Hz, 6.0 Hz, 2H), 7.95-7.94 (m, 1H), 7.80 (br s, 1H), 7.27-7.23 (t, J=8.1 Hz, 2H), 7.14-7.13 (d, J=3.7 Hz, 1H), 3.39-3.38 (d, J=6.1 Hz, 2H), 2.87-2.83 (t, J=7.5 Hz, 2H), 2.18-2.14 (t, J=7.2 Hz, 2H), 1.88-1.81 (m, 2H), 1.31 (s, 6H). MS (ESI) m/z: Calculated for C₂₂H₂₂F₄N₄O₂S: 482.14; found: 481.3 (M−H)⁻.

### Example 16

### 4-Methoxy-4-oxobutanoic acid

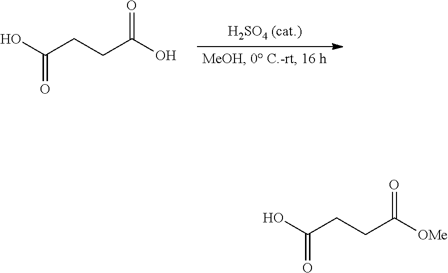

A catalytic amount of conc. H₂SO₄ (4.0 mL) was added to a solution of succinic acid (40 g, 0.34 mol) in dry MeOH (400 mL) at 0° C. and the resulting mixture was stirred at room temperature for 16 h. The reaction mixture was then concentrated under reduced pressure and neutralized with saturated NaHCO₃ solution. The aqueous layer was washed with hexane, acidified to pH ˜2-3 using 6N HCl, and extracted with CH₂Cl₂. The combined extracts were dried over anhydrous sodium sulfate and concentrated under reduced pressure. The crude product was purified by column chromatography (silica 60-120 mesh, eluant 25-30% EtOAc in petroleum ether) to get 4-methoxy-4-oxobutanoic acid (2.9 g, yield 6%). ¹H NMR (400 MHz, CDCl₃) δ 3.71 (s, 3H), 2.72-2.69 (m, 2H), 2.66-2.62 (m, 2H).

### Methyl 5-chloro-4-oxopentanoate

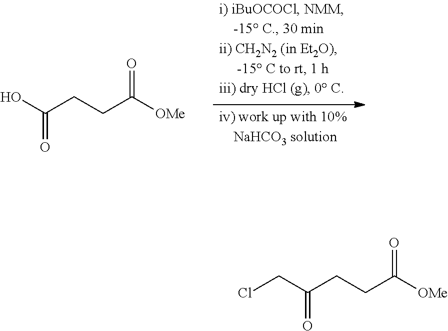

NMM (2.41 mL, 21.95 mmol) was added dropwise to a solution of 4-methoxy-4-oxobutanoic acid (2.9 g, 21.95 mmol) in dry THF (60 mL), followed by isobutyl chloroformate (3.13 mL, 24.15 mmol) at −15° C. and the reaction mixture was stirred for 30 min maintaining the same temperature. Freshly prepared diazomethane in Et₂O (˜100 mL) was added to the reaction mixture at −15° C. and the mixture was then allowed to warm up to room temperature and stirred for another 1 h. After completion of the reaction (monitored by TLC, eluant petroleum ether:EtOAc 1:1 v/v) dry HCl (g) was purged through the reaction mixture for 10 min (till yellow color of the solution disappeared). The reaction mixture was then neutralized with saturated NaHCO₃ solution and extracted with EtOAc. The combined extracts were dried over anhydrous sodium sulfate and concentrated under reduced pressure. The crude product was purified by column chromatography (silica 60-120 mesh, eluant 30% EtOAc in petroleum ether) to get methyl 5-chloro-4-oxopentanoate (1.7 g, yield 47%). ¹H NMR (300 MHz, CDCl₃) δ 4.16 (s, 2H), 3.69 (s, 3H), 2.93-2.88 (m, 2H), 2.69-2.64 (m, 2H).

### Methyl 3-(2-(4-fluorophenyl)oxazol-4-yl)propanoate

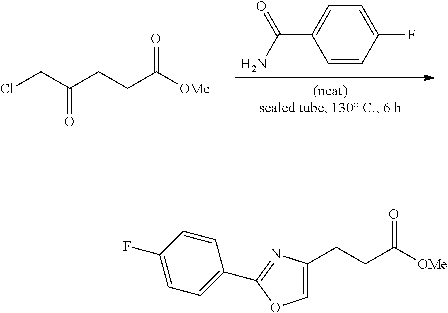

A mixture of 4-fluorobenzamide (1.1 g, 7.91 mmol) and methyl 5-chloro-4-oxopentanoate (1.56 g, 9.48 mmol) was heated to 130° C. for 6 h in a sealed tube. Reaction mixture was cooled to room temperature and the organic product was extracted with CH₂Cl₂. The combined extracts were dried over anhydrous sodium sulfate and concentrated under reduced pressure. The crude product was purified by column chromatography (silica gel 60-120 mesh, eluent 10% EtOAc in petroleum ether) to afford methyl 3-(2-(4-fluorophenyl)oxazol-4-yl)propanoate (780 mg, yield 39%). ¹H NMR (300 MHz, CDCl₃) δ 8.03-7.98 (dd, J=8.7 Hz, 5.4 Hz, 2H), 7.46 (s, 1H), 7.16-7.11 (t, J=8.7 Hz, 2H), 3.70 (s, 3H), 2.95-2.90 (m, 2H), 2.76-2.71 (m, 2H). MS (ESI) m/z: Calculated for C₁₃H₁₂FNO₃: 249.08; found: 250.0 (M+H)⁺.

### 3-(2-(4-Fluorophenyl)oxazol-4-yl)propanoic acid

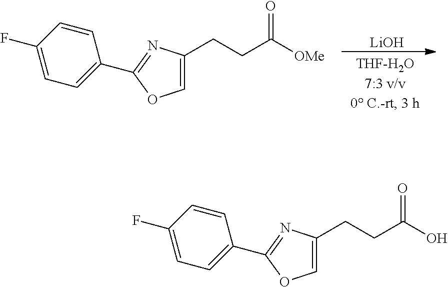

This compound was synthesized from methyl 3-(2-(4-fluorophenyl)oxazol-4-yl)propanoate as described in example 13 step 7 (600 mg, crude) and it was carried through without further purification. ¹H NMR (300 MHz, CDCl₃) δ 8.03-7.99 (m, 2H), 7.49 (s, 1H), 7.18-7.12 (t, J=8.7 Hz, 2H), 2.96-2.91 (m, 2H), 2.82-2.77 (m, 2H). MS (ESI) m/z: Calculated for C₁₂H₁₀FNO₃: 235.06; found: 235.9 (M+H)⁺.

### N-(3-Cyanopropyl)-3-(2-(4-fluorophenyl)oxazol-4-yl)propanamide

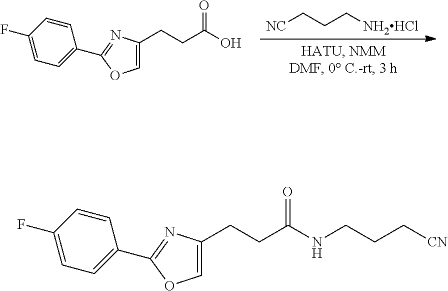

This compound was synthesized from 4-aminobutyronitrile hydrochloride and 3-(2-(4-fluorophenyl)oxazol-4-yl)propanoic acid as described in example 1 step 5 (510 mg, yield 66%). ¹H NMR (400 MHz, CDCl₃) δ 8.00-7.97 (m, 2H), 7.47 (s, 1H), 7.16-7.12 (t, J=8.7 Hz, 2H), 3.39-3.34 (m, 2H), 2.95-2.88 (m, 2H), 2.62-2.58 (t, J=7.0 Hz, 2H), 2.35-2.31 (t, J=7.2 Hz, 2H), 1.87-1.83 (m, 2H). MS (ESI) m/z: Calculated for C₁₆H₁₆FN₃O₂: 301.12; found: 302.1 (M+H)⁺.

### N-(4-Amino-4-(hydroxyimino)butyl)-3-(2-(4-fluorophenyl)oxazol-4-yl)propanamide

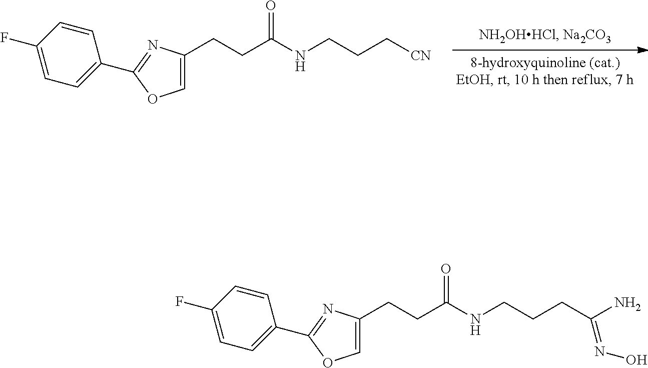

This compound was synthesized from N-(3-cyanopropyl)-3-(2-(4-fluorophenyl)oxazol-4-yl)propanamide as described in example 1 step 6 (150 mg, crude) and it was carried through without further purification. MS (ESI) m/z: Calculated for C₁₆H₁₉FN₄O₃: 334.14; found: 335.2 (M+H)⁺.

### 3-(2-(4-Fluorophenyl)oxazol-4-yl)-N-(3-(5-(trifluoromethyl)-1,2,4-oxadiazol-3-yl)propyl)propanamide

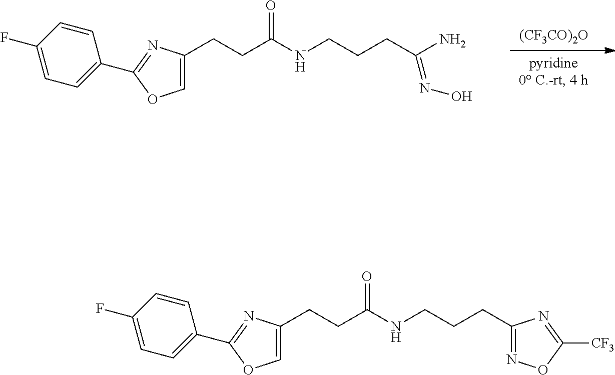

This compound was synthesized from N-(4-amino-4-(hydroxyimino)butyl)-3-(2-(4-fluorophenyl)oxazol-4-yl)propanamide as described in example 1 step 7 (30 mg, yield 16%). ¹H NMR (400 MHz, CDCl₃) δ 8.00-7.97 (dd, J=8.8 Hz, 5.3 Hz, 2H), 7.49 (s, 1H), 7.16-7.11 (t, J=8.8 Hz, 2H), 6.06 (br s, 1H), 3.39-3.34 (m, 2H), 2.94-2.91 (t, J=6.9 Hz, 2H), 2.84-2.80 (t, J=7.7 Hz, 2H), 2.62-2.58 (t, J=7.2 Hz, 2H), 2.01-1.94 (m, 2H). MS (ESI) m/z: Calculated for C₁₈H₁₆F₄N₄O₃: 412.12; found: 413.2 (M+H)⁺.

### Example 17

### 2-(3-(4-Fluorophenyl)-1H-1,2,4-triazol-5-yl)acetonitrile

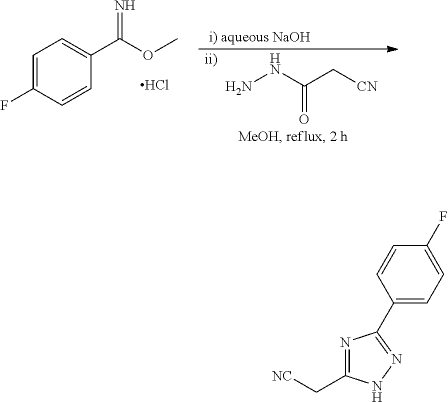

This compound was synthesized from methyl 4-fluorobenzimidate hydrochloride and 2-cyanoacetohydrazide as described in example 15 step 1 (450 mg, yield 56%). ¹H NMR (300 MHz, CDCl₃) δ 7.95-7.91 (dd, J=8.9 Hz, 5.2 Hz, 2H), 7.22-7.16 (t, J=8.7 Hz, 2H), 3.96 (s, 2H). MS (ESI) m/z: Calculated for C₁₀H₇FN₄: 202.07; found: 200.0 (M−H)⁻.

### 2-(3-(4-Fluorophenyl)-1H-1,2,4-triazol-5-yl)ethanamine

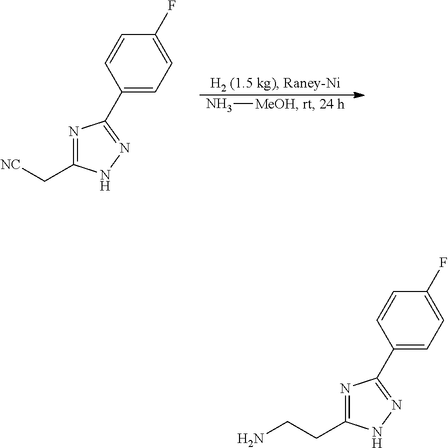

This compound was synthesized from 2-(3-(4-fluorophenyl)-1H-1,2,4-triazol-5-yl)acetonitrile as described in example 4 step 4 (100 mg, crude) and it was carried through without further purification. MS (ESI) m/z: Calculated for C₁₀H₁₁FN₄: 206.10; found: 207.0 (M+H)⁺.

### N-(2-(3-(4-Fluorophenyl)-1H-1,2,4-triazol-5-yl)ethyl)-4-(5-(2,2,2-trifluoroacetyl)thiophen-2-yl)butanamide

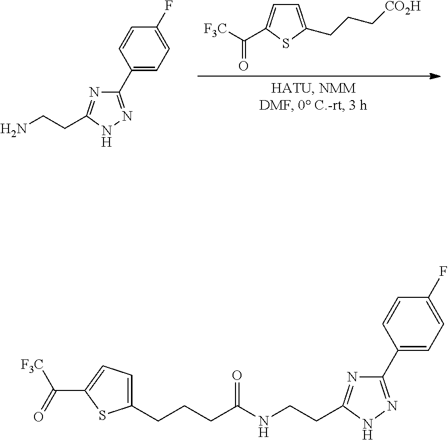

This compound was synthesized from 2-(3-(4-fluorophenyl)-1H-1,2,4-triazol-5-yl)ethanamine and 4-(5-(2,2,2-trifluoroacetyl)thiophen-2-yl)butanoic acid as described in example 1 step 5 (65 mg, yield 38%). ¹H NMR (400 MHz, DMSO-d₆) δ 13.47 (br s, 1H), 8.02-7.99 (m, 2H), 7.94-7.93 (m, 1H), 7.70 (m, 1H), 7.24-7.21 (m, 2H), 7.15-7.14 (d, J=4.0 Hz, 1H), 3.50-3.45 (m, 2H), 2.95-2.92 (m, 4H), 2.20-2.16 (m, 2H), 1.96-1.89 (m, 2H). MS (ESI) m/z: Calculated for C₂₀H₁₈F₄N₄O₂S: 454.11; found: 453.3 (M−H)⁻.

### Example 18

### N-(4-(Dimethylamino)-2-(2-(4-fluorophenyl)oxazol-4-yl)butyl)-4-(5-(2,2,2-trifluoroacetyl)thiophen-2-yl)butanamide

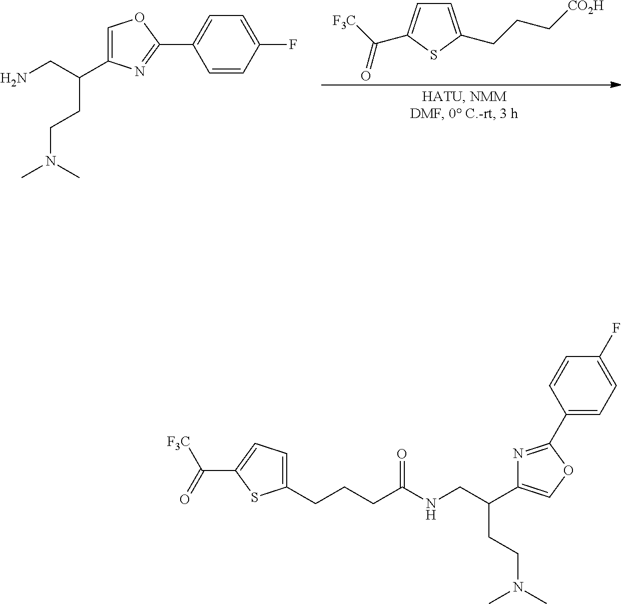

This compound was synthesized from 3-(2-(4-fluorophenyl)oxazol-4-yl)-N1,N1-dimethylbutane-1,4-diamine and 4-(5-(2,2,2-trifluoroacetyl)thiophen-2-yl)butanoic acid as described in example 1 step 5 (32 mg, yield 16%). ¹H NMR (400 MHz, DMSO-d₅, 80° C.) δ 8.01-7.98 (dd, J=9.0 Hz, 5.3 Hz, 2H), 7.93 (m, 2H), 7.70-7.67 (m, 1H), 7.34-7.29 (t, J=8.9 Hz, 2H), 7.15-7.14 (d, J=4.0 Hz, 1H), 3.39-3.34 (m, 2H), 2.94-2.91 (m, 3H), 2.82-2.76 (m, 2H), 2.53 (s, 6H), 2.21-2.17 (m, 2H), 1.96-1.90 (m, 4H). MS (ESI) m/z: Calculated for C₂₅H₂₇F₄N₃O₃S: 525.17; found: 524.4 (M−H)⁻.

### Example 19

### N-((4-(2-(4-Fluorophenyl)oxazol-4-yl)-1-methylpiperidin-4-yl)methyl)-4-(5-(2,2,2-trifluoroacetyl)thiophen-2-yl)butanamide

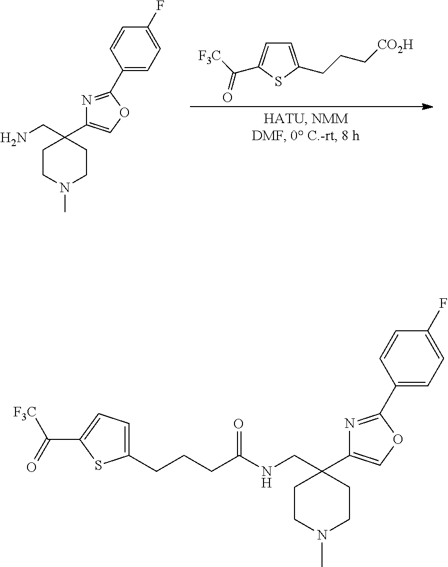

This compound was synthesized from (4-(2-(4-fluorophenyl)oxazol-4-yl)-1-methylpiperidin-4-yl)methanamine and 4-(5-(2,2,2-trifluoroacetyl)thiophen-2-yl)butanoic acid as described in example 1 step 5 (35 mg, yield 13%). ¹H NMR (400 MHz, DMSO-d₅, 80° C.) δ 8.00-7.97 (m, 2H), 7.94-7.93 (m, 1H), 7.86 (s, 1H), 7.32-7.27 (t, J=8.8 Hz, 3H), 7.13-7.12 (d, J=4.0 Hz, 1H), 3.31-3.29 (d, J=6.4 Hz, 2H), 2.92-2.88 (t, J=7.5 Hz, 2H), 2.59-2.54 (m, 2H), 2.19-2.13 (m, 7H), 2.02-1.98 (m, 2H), 1.91-1.85 (m, 2H), 1.76-1.70 (m, 2H). MS (ESI) m/z: Calculated for C₂₆H₂₇F₄N₃O₃S: 537.17; found: 536.6 (M−H)⁻.

### Pharmaceutical Compositions

### Example A

Tablets are prepared using conventional methods and are formulated as follows:

| Ingredient | Amount per tablet |
|---|---|
| Compound of Example I | 5 mg |
| Microcrystalline cellulose | 100 mg |
| Lactose | 100 mg |
| Sodium starch glycollate | 30 mg |
| Magnesium stearate | 2 mg |
| Total | 237 mg |

### Example B

Capsules are prepared using conventional methods and are formulated as follows:

| Ingredient | Amount per tablet |
|---|---|
| Compound of Example 3 | 15 mg |
| Dried starch | 178 mg |
| Magnesium stearate | 2 mg |
| Total | 195 mg |

### Histone Deacetylase 9 (HDAC9) Inhibition Assay:

Novel histone deacetylase 9 (HDAC9) inhibitors were characterized in an in vitro biochemical functional assay. The assay measures the increased fluorescent signal due to deacetylation, by HDAC9, of a fluorogenic substrate. The commercial available substrate is Class IIa HDAC-specific and contains an acetylated lysine residue and would releases the fluorescent signal upon trypsin cleavage after deacetylation.

Specifically, test compounds diluted to various concentrations in 100% DMSO are first dispensed into 384-well assay plates. Recombinant HDAC9 isoform 4 (purchased from BPS Bioscience) in complete assay buffer (50 mM Tris-HCl, pH 8.0, 137 mM NaCl, 2.7 mM KCl, 1 mM MgCl₂, 0.05% BSA & 0.005% Tween 20) were then added to each well (5 uL/well) using Multidrop Combi (Thermo Scientific), followed by 5 uL/well substrate (purchased from BPS Bioscience, 4.5 uM final). After 45 minutes incubation at room temperature, 10 uL 2× developer solution (assay buffer with 40 uM Trypsin and 20 uM Trichostatin A) was added. The plates were then incubated 1 hour at room temperature before reading in fluorescent intensity mode at 450 nm in an Envision (Perkin Elmer) plate reader. Percent Inhibition of HDAC9 activity by compounds in each test wells was calculated by normalizing to fluorescent signal in control wells containing DMSO only. The pIC50s value of test compounds were calculated from non-linear curve fitting, using ActivityBase5 data analysis tool (IDBS), from 11 point 3× dilution series starting from 100 uM final compound concentration.

For dose response experiments, normalized data were fit by ABASE/XC50 using the equation y=a+(b−a)/(1+(10̂×/10̂c)̂d), where a is the minimum % activity, b is the maximum % activity, c is the pIC₅₀, d is the Hill slope.

### Biological Results

The pIC₅₀s are averaged to determine a mean value, for a minimum of 2 experiments. As determined using the above method, the compounds of Examples 1-19 exhibited a pIC₅₀ greater than 4.0. For instance, the compounds of Examples 3, 4, 5 and 18 inhibited HDAC9 in the above method with a mean pIC₅₀ of >6.

### REFERENCES

US 20060269559, U.S. Pat. No. 7,521,044, WO2007084775
“Deacetylase inhibition promotes the generation and function of regulatory T cells,” R. Tao, E. F. de Zoeten, E. O'zkaynak, C. Chen, L. Wang, P. M. Porrett, B. Li, L. A. Turka, E. N. Olson, M. I. Greene, A. D. Wells, W. W. Hancock, Nature Medicine, 13 (11), 2007.
“Expression of HDAC9 by T Regulatory Cells Prevents Colitis in Mice,” E. F. de Zoeten, L. Wang, H. Sai, W. H. Dillmann, W. W. Hancock, Gastroenterology. 2009 Oct. 28.
“Immunomodulatory effects of deacetylase inhibitors: therapeutic targeting of FOXP3+ regulatory T cells,” L. Wang, E. F. de Zoeten, M. I. Greene and W. W. Hancock, Nature Review Drug Discovery. 8(12):969-81, 2009 and references therein.
“HDAC7 targeting enhances FOXP3+Treg function and induces long-term allograft survival,” L. Wang, et al., Am. J. Transplant 9, S621 (2009).
“Selective class II HDAC inhibitors impair myogenesis by modulating the stability and activity of HDAC-MEF2 complexes,” A. Nebbioso, F. Manzo, M. Miceli, M. Conte, L. Manente, A. Baldi, A. De Luca, D. Rotili, S. Valente, A. Mai, A. Usiello, H. Gronemeyer, L. Altucci, EMBO reports 10 (7), 776-782, 2009. and references therein.
“Myocyte Enhancer Factor 2 and Class II Histone Deacetylases Control a Gender-Specific Pathway of Cardioprotection Mediated by the Estrogen Receptor,” E. van Rooij, J. Fielitz, L. B. Sutherland, V. L. Thijssen, H. J. Crijns, M. J. Dimaio, J. Shelton, L. J. De Windt, J. A. Hill, E. N. Olson, Circulation Research, January 2010.

1-50. (canceled)

51. A compound according to Formula I: 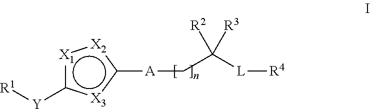
wherein:
R¹ is halo(C₁-C₄)alkyl, wherein said halo(C₁-C₄)alkyl contains at least 2 halo groups (R¹ is di-halo(C₁-C₄)alkyl);
Y is a bond and X₁ is O, X₂ is N or CH and X₃ is N or NH, or Y is —C(O)— and X₁ and X₂ are CH or N, X₃ is O or S, or Y is —C(O)— and X₁ is O, X₂ is CH or N, and X₃ is CH or N;
n is 0-4;
A is —C(═O)NR^{X}—, —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —((C₁-C₆)alkyl)SO₂—, —SO₂NR^{X}—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—;
when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,
when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then Z is —C(═O)NR^{X}—, —NR^{X}C(═O)NR^{X}, —SO₂NR^{X}—, —NHCH(CF₃)—, —CH(CF₃)NH—, —CH(CF₃)—, —(C₁-C₄)alkyl-, or —(C₁-C₃)alkyl-NR^{X}—, and
when n is 1-4, R² is selected from —NR^{A}R^{B}, —(C₁-C₄)alkyl-NR^{A}R^{B}, —CONR^{A}R^{B}, —(C₁-C₄)alkyl-CONR^{A}R^{B}, —CO₂H, —(C₁-C₄)alkyl-CO₂H, hydroxyl, hydroxy(C₁-C₄)alkyl-, (C₁-C₃)alkoxy, and (C₁-C₃)alkoxy(C₁-C₄)alkyl-, and R³ is selected from H and optionally substituted (C₁-C₄)alkyl, aryl(C₁-C₄)alkyl-, and (C₃-C₇)cycloalkyl(C₁-C₄)alkyl-,
wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, —NR^{A}R^{A}, —((C₁-C₄)alkyl)NR^{A}R^{A}, and hydroxyl;
or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, and —NR^{Y}C(═O)NR^{Y}R^{Y}; and
L is 5-6 membered heteroaryl or phenyl which is substituted by R⁴ and is optionally further substituted,
wherein when L is further substituted, L is substituted by 1 or 2 substituents independently selected from halogen, cyano and (C₁-C₄)alkyl;
R⁴ is H, (C₁-C₄)alkyl, halo, halo(C₁-C₄)alkyl, (C₁-C₄)alkoxy, ((C₁-C₄)alkyl)((C₁-C₄)alkyl)N(C₁-C₄)alkoxy, ((C₁-C₄)alkyl)((C₁-C₄)alkyl)N(C₁-C₄)alkyl-, (C₁-C₄)haloalkoxy-, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,
wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};
or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from (C₁-C₄)alkyl, halogen, cyano, halo(C₁-C₄)alkyl, (C₁-C₄)alkoxy, (C₁-C₄)alkylthio-, halo(C₁-C₄)alkoxy, hydroxyl, —NR^{A}R^{C} and —((C₁-C₄)alkyl)NR^{A}R^{C};
wherein each R^{A} is independently selected from H and (C₁-C₄)alkyl;
R^{B} is H, (C₁-C₄)alkyl, halo(C₁-C₄)alkyl, —C(═O)(C₁-C₄)alkyl, —C(═O)O(C₁-C₄)alkyl, —C(═O)NH₂, —C(═O)NH(C₁-C₄)alkyl, —C(═O)N((C₁-C₄)alkyl)((C₁-C₄)alkyl), —SO₂(C₁-C₄)alkyl, or R^{A} and R^{B} taken together with the atom to which they are attached form a 4-6 membered heterocyclic ring, optionally containing one additional heteroatom selected from N, O and S and optionally substituted by (C₁-C₄)alkyl;
R^{C} is H, (C₁-C₄)alkyl, phenyl, 5-6 membered heterocycloalkyl, or 5-6 membered heteroaryl, or R^{A} and R^{C} taken together with the atom to which they are attached form a 4-8 membered heterocyclic ring, optionally containing one additional heteroatom selected from N, O and S and optionally substituted by (C₁-C₄)alkyl;
each R^{X} is independently selected from H, (C₁-C₆)alkyl, and optionally substituted (C₂-C₆)alkyl, where said optionally substituted (C₂-C₆)alkyl is optionally substituted by hydroxyl, cyano, amino, (C₁-C₄)alkoxy, (C₁-C₄)alkyl)NH—, or ((C₁-C₄)alkyl)((C₁-C₄)alkyl)N—; and
each R^{Y} is independently selected from H, (C₁-C₄)alkyl, phenyl, and —(C₁-C₄)alkylphenyl;
provided that when Y is —C(O)— and A is —C(═O)NR^{X}— or —SO₂NR^{X}—, then at least one of R² and R³ is not H;
and provided that the compound is not 2,2,2-trifluoro-1-[5-[[methyl(phenylmethyl)amino]methyl]-2-thienyl]-ethanone, 2,2,2-trifluoro-1-[5-[[[(1R)-1-phenylethyl]amino]methyl]-2-thienyl]-ethanone, N-methyl-2-phenyl-N-(2-(5-(trifluoromethyl)-1,2,4-oxadiazol-3-yl)ethyl)acetamide, N-methyl-2-phenyl-N-(2-(5-(trichloromethyl)-1,2,4-oxadiazol-3-yl)ethyl)acetamide, N-[2-[5-(dichloromethyl)-1,2,4-oxadiazol-3-yl]ethyl]-N-methyl-benzeneacetamide, N-[2-(3,4-dimethoxyphenyl)ethyl]-5-(trifluoromethyl)-1,2,4-oxadiazole-3-acetamide, and N-(phenylmethyl)-5-(trifluoromethyl)-1,2,4-oxadiazole-3-methanamine;
or a salt thereof.

52. The compound or salt according to claim 51, wherein R¹ is CHF₂ or CF₃.

53. The compound or salt according to claim 51, wherein when Y is a bond, X₁ is O, and X₂ and X₃ are N.

54. The compound or salt according to claim 51, wherein when Y is —C(O)—, X₁ and X₂ are CH, and X₃ is S.

55. The compound or salt according to claim 51, wherein when Y is —C(O)—, X₁ is O, X₂ and X₃ are CH.

56. The compound or salt according to claim 51, having the formula: 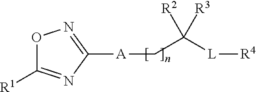
wherein:
R¹ is —CF₃;
n is 0-4;
A is —((C₁-C₆)alkyl)C(═O)NR^{X}—, —((C₁-C₆)alkyl)NR^{X}C(═O)NR^{X}, —((C₁-C₆)alkyl)NR^{X}C(═O)—, —((C₁-C₆)alkyl)SO₂—, —SO₂NR^{X}—, —((C₁-C₆)alkyl)SO₂NR^{X}—, —((C₁-C₆)alkyl)NR^{X}SO₂—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, or —((C₁-C₆)alkyl)NR^{X}—;
when n is 0, R² and R³ are independently selected from H and optionally substituted (C₁-C₄)alkyl, aryl(C₁-C₄)alkyl-, and (C₃-C₇)cycloalkyl(C₁-C₄)alkyl-,
when n is 1-4, R² and R³ are independently selected from H, fluoro, and optionally substituted (C₁-C₄)alkyl, aryl(C₁-C₄)alkyl-, and (C₃-C₇)cycloalkyl(C₁-C₄)alkyl-, wherein, when n is 1, R² is F and R³ is H, then A is —C(═O)NR^{X}—, —((C₁-C₆)alkyl)C(═O)NR^{X}—, —((C₁-C₆)alkyl)NR^{X}C(═O)NR^{X}, —SO₂NR^{X}—, —((C₁-C₆)alkyl)SO₂NR^{X}—, —((C₁-C₆)alkyl)NHCH(CF₃)—, —CH(CF₃)NH—, —((C₁-C₆)alkyl)CH(CF₃)NH—, —CH(CF₃)—, —((C₁-C₆)alkyl)CH(CF₃)—, —(C₁-C₄)alkyl-, or —((C₁-C₆)alkyl)NR^{X}—, and
when n is 1-4, R² is selected from amino, hydroxyl, (C₁-C₄)alkoxy, and R³ is selected from H and optionally substituted (C₁-C₄)alkyl, aryl(C₁-C₄)alkyl-, and (C₃-C₇)cycloalkyl(C₁-C₄)alkyl-,
wherein the aryl, cycloalkyl and each of the (C₁-C₄)alkyl moieties of said optionally substituted (C₁-C₄)alkyl, aryl(C₁-C₄)alkyl-, and (C₃-C₇)cycloalkyl(C₁-C₄)alkyl- of any R² and R³ are optionally substituted by 1, 2 or 3 groups independently selected from halogen, cyano, (C₁-C₄)alkyl, halo(C₁-C₄)alkyl, (C₁-C₄)alkoxy, halo(C₁-C₄)alkoxy, halogen, NR^{A}R^{A}, —((C₁-C₄)alkyl)NR^{A}R^{A}, (C₁-C₄)alkoxy, hydroxyl, cyano, halo(C₁-C₄)alkyl, and halo(C₁-C₄)alkoxy;
or R² and R³ taken together with the atom to which they are connected form an optionally substituted 4, 5, 6, or 7 membered cycloalkyl or heterocycloalkyl group, wherein said heterocycloalkyl group contains 1 or 2 heteroatoms independently selected from N, O and S and said optionally substituted cycloalkyl or heterocycloalkyl group is optionally substituted by 1, 2 or 3 substituents independently selected from (C₁-C₄)alkyl, halo(C₁-C₄)alkyl, halogen, cyano, aryl(C₁-C₄)alkyl-, (C₃-C₇)cycloalkyl(C₁-C₄)alkyl-, —OR^{Y}, —NR^{Y}R^{Y}, —C(═O)OR^{Y}, —C(═O)NR^{Y}R^{Y}, —NR^{Y}C(═O)R^{Y}, —SO₂NR^{Y}R^{Y}, —NR^{Y}SO₂R^{Y}, —OC(═O)NR^{Y}R^{Y}, —NR^{Y}C(═O)OR^{Y}, —NR^{Y}C(═O)NR^{Y}R^{Y}; and
L is 5-6 membered heteroaryl or phenyl which is substituted by R⁴ and is optionally further substituted,
wherein when L is further substituted, L is substituted by 1 or 2 substituents independently selected from halogen, cyano and (C₁-C₄)alkyl;
R⁴ is H, (C₁-C₄)alkyl, halo(C₁-C₄)alkyl, (C₁-C₄)alkoxy, ((C₁-C₄)alkyl)((C₁-C₄)alkyl)N(C₁-C₄)alkoxy, (C₁-C₄)haloalkyl, (C₁-C₄)alkylamino, optionally substituted (C₃-C₆)cycloalkyl, optionally substituted phenyl, optionally substituted 5-6 membered heterocycloalkyl, or optionally substituted 5-6 membered heteroaryl,
wherein said optionally substituted cycloalkyl, phenyl, heterocycloalkyl or heteroaryl is optionally substituted by 1, 2 or 3 groups independently selected from (C₁-C₄)alkyl, halogen, cyano, halo(C₁-C₄)alkyl, (C₁-C₄)alkoxy, halo(C₁-C₄)alkoxy, hydroxyl, —NR^{A}R^{A} and —((C₁-C₄)alkyl)NR^{A}R^{A};
or L-R⁴, taken together, form a 1,3-benzodioxolyl, 2,3-dihydro-1,4-benzodioxinyl, benzofuranyl, tetrahydroisoquinolyl or isoindolinyl group wherein said benzofuranyl, tetrahydroisoquinolyl or isoindolinyl group is optionally substituted by 1, 2 or 3 groups independently selected from halogen, (C₁-C₄)alkyl, cyano, halo(C₁-C₄)alkoxy, (C₁-C₄)alkoxy, and halo(C₁-C₄)alkyl;
wherein each R^{A} is independently selected from H and (C₁-C₄)alkyl;
each R^{X} is independently selected from H, (C₁-C₆)alkyl, or optionally substituted (C₂-C₆)alkyl, where said optionally substituted (C₂-C₆)alkyl is optionally substituted by hydroxyl, cyano, amino, (C₁-C₄)alkoxy, ((C₁-C₄)alkyl)NH—, or ((C₁-C₄)alkyl)((C₁-C₄)alkyl)N—; and
each R^{Y} is independently selected from H, (C₁-C₄)alkyl, phenyl, and —(C₁-C₄)alkylphenyl;
or a salt, particularly a pharmaceutically acceptable salt, thereof.

57. The compound or salt according to claim 51, wherein A is —((C₁-C₄alkyl)C(═O)NR^{X}—, —((C₁-C₄)alkyl)SO₂NR^{X}—, —((C₁-C₄)alkyl)NR^{X}C(═O)NR^{X}— or —((C₁-C₄)alkyl)NR^{X}C(═O)—

58. The compound or salt according to claim 51, wherein each R^{X} is independently selected from H, (C₁-C₄)alkyl, or optionally substituted (C₂-C₄)alkyl, where said optionally substituted (C₂-C₄)alkyl is optionally substituted by hydroxyl, cyano, amino, (C₁-C₄)alkoxy, (C₁-C₄)alkyl)NH—, or ((C₁-C₂)alkyl)((C₁-C₂)alkyl)N—.

59. The compound or salt according to claim 51, wherein each R^{X} is independently H or methyl.

60. The compound or salt according to claim 51, wherein n is 0 or 1.

61. The compound or salt according to claim 51, wherein R² and R³ taken together with the atom to which they are connected form an optionally substituted 4, 5 or 6 membered cycloalkyl or heterocycloalkyl group, wherein said heterocycloalkyl group contains 1 heteroatom selected from N and O and said optionally substituted cycloalkyl or heterocycloalkyl group is optionally substituted by a substituent selected from (C₁-C₄)alkyl, aryl(C₁-C₂)alkyl-, and (C₃-C₆)cycloalkyl(C₁-C₂)alkyl-.

62. The compound or salt according to claim 51, wherein R² and R³ are independently selected from optionally substituted (C₁-C₄)alkyl, phenyl(C₁-C₂)alkyl-, and (C₃-C₆)cycloalkyl(C₁-C₂)alkyl-.

63. The compound or salt according to claim 51, wherein one of R² and R³ is H, and the other of R² and R³ is selected from optionally substituted (C₁-C₄)alkyl, phenyl(C₁-C₂)alkyl-, and (C₃-C₆)cycloalkyl(C₁-C₂)alkyl-, provided that when Y is —C(O)— A is not —C(═O)NR^{X}— or —SO₂NR^{X}—.

64. The compound or salt according to claim 51, wherein R² and R³ are each methyl.

65. The compound or salt according to claim 51, wherein L is 5-6 membered heteroaryl or phenyl group which is substituted by R⁴ and is optionally further substituted by 1 substituent selected from halogen, cyano and (C₁-C₄)alkyl.

66. The compound or salt according to claim 51, wherein R⁴ is H, (C₁-C₄)alkyl, (C₁-C₄)haloalkyl, (C₁-C₄)alkoxy, ((C₁-C₄)alkyl)((C₁-C₄)alkyl)N(C₁-C₄)alkoxy, (C₁-C₄)alkylamino, optionally substituted phenyl, or optionally substituted 5-6 membered heteroaryl.

67. The compound or salt according to claim 51, wherein
A is —((C₂-C₄)alkyl)C(═O)NR^{X}— or —((C₂-C₄)alkyl)NR^{X}C(═O)—;
n is 1-3;
R² and R³ taken together with the atom to which they are connected form an optionally substituted 4, 5 or 6 membered cycloalkyl or heterocycloalkyl group, wherein said heterocycloalkyl group contains 1 heteroatom selected from N and O and said optionally substituted cycloalkyl or heterocycloalkyl group is optionally substituted by a substituent selected from (C₁-C₄)alkyl, aryl(C₁-C₂)alkyl-, and (C₃-C₆)cycloalkyl(C₁-C₂)alkyl-;
or R² is selected from H and optionally substituted (C₁-C₄)alkyl, phenyl(C₁-C₂)alkyl-, and (C₃-C₆)cycloalkyl(C₁-C₂)alkyl- and R³ is selected from H and methyl,
or R² is (C₁-C₂)alkylamino, ((C₁-C₂)alkyl)((C₁-C₂)alkyl)amino, amino(C₁-C₃)alkyl, (C₁-C₂)alkylamino(C₁-C₃)alkyl, or ((C₁-C₂)alkyl)((C₁-C₂)alkyl)amino(C₁-C₃)alkyl, and R³ is H or (C₁-C₂)alkyl,
or R² is hydroxyl and R³ is H or methyl;
L is thiazolyl, thienyl, triazolyl, oxazolyl or phenyl which is substituted by R⁴ and is optionally further substituted by a methyl group; and
R⁴ is H, methyl, phenyl, 4-chlorophenyl, 4-fluorophenyl, 3,5-difluorophenyl, 4-cyanophenyl, 4-methoxyphenyl, pyrid-2-yl, pyrid-3-yl, or pyrid-4-yl.

68. A compound which is:
N-((4-(4-phenylthiazol-2-yl)tetrahydro-2H-pyran-4-yl)methyl)-4-(5-(trifluoromethyl)-1,2,4-oxadiazol-3-yl)butanamide,
4-(5-(difluoromethyl)-1,2,4-oxadiazol-3-yl)-N-(2-(2-(4-fluorophenyl)oxazol-4-yl)-2-methylpropyl)butanamide,
N-(2-(2-(4-fluorophenyl)oxazol-4-yl)-2-methylpropyl)-4-(5-(trifluoromethyl)-1,2,4-oxadiazol-3-yl)butanamide,
N-(4-(dimethylamino)-2-(2-(4-fluorophenyl)oxazol-4-yl)butyl)-4-(5-(trifluoromethyl)-1,2,4-oxadiazol-3-yl)butanamide,
N-((4-(2-(4-fluorophenyl)oxazol-4-yl)-1-methylpiperidin-4-yl)methyl)-4-(5-(trifluoromethyl)-1,2,4-oxadiazol-3-yl)butanamide,
N-(2-(2-(4-fluorophenyl)oxazol-4-yl)-2-methylpropyl)-2,2-dimethyl-4-(5-(trifluoromethyl)-1,2,4-oxadiazol-3-yl)butanamide,
N-(2-(2-(4-fluorophenyl)oxazol-4-yl)ethyl)-4-(5-(trifluoromethyl)-1,2,4-oxadiazol-3-yl)butanamide,
N-((4-(2-(4-chlorophenyl)thiazol-4-yl)-1-methylpiperidin-4-yl)methyl)-4-(5-(trifluoromethyl)-1,2,4-oxadiazol-3-yl)butanamide,
N-(2-(2-(4-fluorophenyl)oxazol-4-yl)-2-hydroxyethyl)-4-(5-(trifluoromethyl)-1,2,4-oxadiazol-3-yl)butanamide,
4-(5-(difluoromethyl)-1,2,4-oxadiazol-3-yl)-N-(4-(dimethylamino)-2-(2-(4-fluorophenyl)oxazol-4-yl)butyl)butanamide,
N-(2-(2-(4-fluorophenyl)oxazol-4-yl)-2-methylpropyl)-3-(5-(trifluoromethyl)-1,2,4-oxadiazol-3-yl)propane-1-sulfonamide,
N-(3-(2-(4-fluorophenyl)oxazol-4-yl)-3-hydroxypropyl)-3-(5-(trifluoromethyl)-1,2,4-oxadiazol-3-yl)propanamide,
N-(2-(2-(4-fluorophenyl)oxazol-4-yl)-2-methylpropyl)-4-(5-(2,2,2-trifluoroacetyl)thiophen-2-yl)butanamide,
N-(2-(2-(4-fluorophenyl)oxazol-4-yl)ethyl)-4-(5-(2,2,2-trifluoroacetyl)thiophen-2-yl)butanamide,
N-(2-(3-(4-fluorophenyl)-1H-1,2,4-triazol-5-yl)-2-methylpropyl)-4-(5-(2,2,2-trifluoroacetyl)thiophen-2-yl)butanamide,
3-(2-(4-fluorophenyl)oxazol-4-yl)-N-(3-(5-(trifluoromethyl)-1,2,4-oxadiazol-3-yl)propyl)propanamide,
N-(2-(3-(4-fluorophenyl)-1H-1,2,4-triazol-5-yl)ethyl)-4-(5-(2,2,2-trifluoroacetyl)thiophen-2-yl)butanamide,
N-(4-(dimethylamino)-2-(2-(4-fluorophenyl)oxazol-4-yl)butyl)-4-(5-(2,2,2-trifluoroacetyl)thiophen-2-yl)butanamide, or
N-((4-(2-(4-fluorophenyl)oxazol-4-yl)-1-methylpiperidin-4-yl)methyl)-4-(5-(2,2,2-trifluoroacetyl)thiophen-2-yl)butanamide,
or a pharmaceutically acceptable salt thereof.

69. A pharmaceutical composition comprising the compound or salt according to claim 51 and one or more pharmaceutically-acceptable excipients.

70. A method of treatment of an HDAC-mediated disease or disorder comprising administering a therapeutically effective amount of the compound or salt according to claim 51 to a human in need thereof.
